(19) United States
(12) Patent Application Publication
(10) Pub. No.: US 2023/0310602 A1
(43) Pub. Date: Oct. 5, 2023

(54) COMPOSITIONS AND METHODS FOR TREATMENT OF INFLAMMATORY DISORDERS

(71) Applicant: Robert Sackstein, Pinecrest, FL (US)

(72) Inventor: Robert Sackstein, Pinecrest, FL (US)

(21) Appl. No.: 18/056,371

(22) Filed: Nov. 17, 2022

Related U.S. Application Data
(63) Continuation of application No. PCT/US21/32859, filed on May 18, 2021.
(60) Provisional application No. 63/026,629, filed on May 18, 2020.

(51) Int. Cl. A61K 39/00; A61P 37/06

(52) U.S. Cl. CPC A61K 39/4622; A61K 39/461; A61K 39/4634; A61K 39/4637; A61P 37/06; A61K 39/4635

(57) ABSTRACT

The present disclosure provides, inter alia, compositions, cell populations and pharmaceutical compositions and methods useful for the treatment of inflammatory diseases or disorders. In some embodiments, the compositions, cell populations and pharmaceutical compositions and methods comprise a population of CD44⁺ cells modified ex vivo via treatment with a CD44 ligand for a period of time sufficient to prime the cells to produce elevated levels of one or more anti-inflammatory or immunomodulatory molecules relative to a native populations of CD44⁺ cells. In some embodiments, the compositions, cell populations and pharmaceutical compositions and methods comprise a population of CD44⁺ cells modified ex vivo via a treatment that is effective to target cells to sites of inflammation.

### CROSS REFERENCE TO RELATED APPLICATIONS

The present application is a continuation of PCT/US21/32859 filed on May 18, 2021, which claims benefit to U.S. Provisional Patent Application Ser. No. 63/026,629, filed on May 18, 2020, which is incorporated by reference herein in its entirety.

### INCORPORATION BY REFERENCE OF SEQUENCE LISTING

This application contains references to amino acids and/or nucleic acid sequences that have been filed concurrently herewith as sequence listing text file “SequenceListingPCT.txt,” file size of 35 KB, created on May 18, 2020. The aforementioned sequence listing is hereby incorporated by reference in its entirety.

### FIELD OF INVENTION

The present invention discloses, inter alia, compositions and methods for improvement of the therapeutic usefulness of mesenchymal stem cells (MSCs), particularly for, but not limited to, the treatment of inflammatory conditions.

### BACKGROUND OF THE INVENTION

### Mesenchymal Stem Cells

Mesenchymal stem cells (MSCs) are multipotent cells most recognized for their ability to differentiate into connective tissue cells such as osteoblasts, adipocytes, chondrocytes, and fibroblasts. It is believed that every tissue of the body contains a variable amount of MSCs, but the true level of these cells in any given tissue site cannot be gauged because at present there are no cell surface markers that can uniquely identify an MSC per se. As such, one cannot know with any certainty whether a given tissue possesses a sufficient, threshold amount of MSCs so as to be able to perform, in vivo, any one or more of its functions as suggested by in vitro studies. Indeed, precisely because there are no distinctive markers to purify the cells directly from tissue sources, these cells can only be isolated by ex vivo culture conditions, characteristically accomplished by way of their propensity to adhere to tissue culture plastic surfaces. The adherent cells are then culture-expanded to whatever quantity may be desired for a given clinical indication. One can then release the cells from the support surfaces for immediate use or can preserve (e.g., cryopreserve) the cells for future application(s). To date, MSCs derived from human bone marrow have been used most commonly in clinical applications, but MSCs can be procured from a variety of human tissues and organs including, but not limited to, bone marrow, adipose tissue, umbilical cord, blood, dental pulp, placenta, skin, muscle, and lung.

MSCs have the ability to mediate tissue repair by release of biologic factors that drive functional recovery of cells within their milieu (e.g., VEGF, EGF, etc.) and also directly contribute to remodeling through their ability to proliferate and differentiate into diverse cell types. Thus, these undifferentiated multipotent cells are key players in the maintenance of tissue integrity.

It has been found by in vitro studies that MSCs also possess potent anti-inflammatory and immunomodulatory properties, and MSC-based therapies have been used with variable success in treatment of several immune-mediated diseases such as diabetes, rheumatoid arthritis, inflammatory bowel disease, and acute graft-versus-host disease (aGvHD). To date, however, it has not been determined whether the presence of MSCs within the inflamed tissue itself is needed for their beneficial anti-inflammatory/immunomodulatory and/or tissue-reparative effect(s), or if the MSCs exert their immunobiologic effects by “remote control”, i.e., within distant sites such as lymphoid tissues/organs. The latter has been presumed, because MSCs are sessile cells, uniformly lacking expression of “homing receptors” (such as E-selectin ligands) that are critical for migration of blood-borne cells to endothelial beds (and commensurate extravasation) at sites of tissue injury/inflammation. Indeed, though MSCs are frequently administered systemically, little attention has been paid to the fact that these cells ineffectively enter inflamed tissue(s): MSCs are natively devoid of ligands for the vascular endothelial lectin known as “E-selectin”, the principal endothelial molecule that functions as a beacon for attracting cells in blood flow to inflamed tissues; consequently, MSCs have limited ability to engage inflamed vascular endothelium under hemodynamic shear conditions. Because of this inability of circulating MSCs to migrate into inflammatory sites and then efflux/extravasate from blood, it has been generally held that MSCs do not need to colonize the affected tissue proper in order to modulate the inflammatory response.

As described in greater detail below, E-selectin is a lectin that binds to a sialofucosylated lactosaminyl glycan motif known as “sialylated Lewis X” (sLe^{X}: NeuAc-α(2,3)-Gal-β(1,4)-[Fuc-α(1,3)]-GlcNAc-α1-R). Human mesenchymal stem cells (hMSCs) are known to natively display sialylated terminal Type 2 lactosaminyl glycans (thus possessing glycosyltransferases necessary to create this structure), but are natively deficient in the sLe^{X} decorations of CD44 that create the specialized sialofucosylated CD44 glycoform known as “Hematopoietic Cell E-/L-selectin Ligand” (HCELL). HCELL is the most potent E-selectin and L-selectin ligand displayed on human cells. It is now clear that MSCs derived from many tissue sources (e.g., bone marrow, adipose tissue, umbilical cord) have a glycosignature in which sialylated type 2 lactosamines are displayed on a CD44 backbone. Treatment of MSCs with α(1,3)-fucosyltransferases (e.g., FTVI or FTVII) in the presence of the nucleotide sugar donor GDP-fucose converts CD44 into HCELL. This cell surface glycan engineering technology is called “glycosyltransferase programmed stereosubstitution” (GPS). Following exofucosylation, HCELL expression lasts about 24-48 hours, gradually reversing to the endogenous CD44 phenotype by the normal protein turnover of the MSCs surface. CD44 conversion to HCELL endows potent adhesion to E-selectin under fluid shear stress conditions, thus driving interactions on E-selectin-bearing microvessels. Accordingly, GPS-mediated enforced HCELL expression enables homing of MSCs to sites of inflammation/tissue injury, potentiating the use of these cells in cell therapy.

### Immunomodulatory Properties of MSCs

As detailed above, MSCs are multipotent cells distributed throughout many tissues, but, prior to data disclosed herein, there has been no evidence presented that directly indicates that these cells promote immunomodulation in situ (i.e., within the tissues in which they reside). Instead, it has been believed that they impart indirect (i.e., through release of biologic agents, their “secrotome”) tissue reparative properties, an effect that could be exerted by release of the secretome from MSCs that are distant from sites of tissue injury. In vitro studies indicate that culture-expanded MSCs possess potent immunomodulatory properties mediated through a variety of secreted molecules (e.g., transforming growth factor-β (TGFβ), indoleamine 2,3-dioxygenase (IDO), nitric oxide (NO), and prostaglandin E₂ (PGE₂)) and through direct cell-cell contacts (Francois et al., 2012; Nauta and Fibbe, 2007; Ren et al., 2008; Sotiropoulou et al., 2006; Stappenbeck and Miyoshi, 2009; Yanez et al., 2010). However, despite the fact that preclinical and clinical investigations provide evidence of MSC immunomodulation (Cho et al., 2019; Galipeau and Sensebe, 2018; Manieri et al., 2015; Nauta and Fibbe, 2007), knowledge on whether tissue residency is key to this effect and whether microenvironmental components trigger this MSC biology is lacking. Again, a major hurdle to gaining this knowledge is that there are no unique cell surface markers nor gene products that can be used to measure the presence of MSCs within tissue compartments; thus, it is not possible to distinctly quantify MSC colonization within any inflammatory milieu.

### Vascular Delivery of MSCs to Sites of Inflammation

Because culture-expanded MSCs lack molecular effectors of cell migration, when systemically administered, these cells do not efficiently extravasate at inflammatory sites (Ankrum and Karp, 2010). Thus, the ability to directly assess whether MSCs can exert immunomodulation in situ and the mechanistic basis for this process requires a method to program obligatory tissue delivery of MSCs to lesional sites coincident with the onset/progression of immunopathology.

Extravasation of circulating cells is critically dependent on their ability to adhere to vascular endothelial cells with sufficient strength to overcome the shear forces of hemodynamic flow. This process is principally regulated by a family of lectins known as “selectins” (comprised of E-, P-, and L-selectin) that bind to a tetrasaccharide determinant known as “sialylated Lewis X” (sLe^{X}; CD15s). This structure is comprised of a terminal type 2 lactosamine (i.e., galactose (Gal) β(1,4)-linked to N-acetylglucosamine (GlcNAc)), bearing sialic acid (NeuAc) and fucose (Fuc) substitutions: NeuAc-α(2,3)-Gal-β(1,4)-[Fuc-α(1,3)]-GlcNAc-α1-R. MSCs natively lack of display of sLe^{X}, and thus do not express ligands for the selectin, including the endothelial selectin “E-selectin” (CD62E) (Sackstein et al., 2008). However, MSCs uniformly express CD44, a glycoprotein best known for being the principal receptor for hyaluronic acid (HA) (Aruffo et al., 1990). Notably, MSC CD44 is decorated with terminal sialylated type 2 lactosamines (Sackstein, 2016), lacking only the presence of fucose in α(1,3)-linkage to GlcNAc to complete the creation of the sLe^{x} determinant HCELL, a CD44 glycovariant that is a highly potent E-selectin (and L-selectin) ligand. Thus, α(1,3)-exofucosylation of MSC CD44 enforces HCELL expression, programming MSC migration to E-selectin-bearing endothelial beds (Sackstein, 2009). Furthermore, MSCs characteristically display the 31 integrin VLA-4, and engagement of HCELL with vascular E-selectin results in direct activation of VLA-4 in the absence of chemokine signaling; subsequent binding of activated VLA-4 to its endothelial ligand, VCAM-1, leads to firm arrest and extravasation (Thankamony and Sackstein, 2011). Since both E-selectin and VCAM-1 expression is induced by pro-inflammatory cytokines TNF-α and IL-1, and both molecules are consistently found in endothelial beds at sites of immunopathology (Sackstein, 2006; Sloane and Norton, 1993), blood-borne cells expressing both HCELL and VLA-4 are primed to home to inflammatory sites.

### Therapeutic Approaches

Current therapeutic approaches for many immune-mediated diseases are pharmacologic, causing immunosuppression. Though these agents may offer therapeutic benefits, they are usually not curative and are associated with significant short-term and long-term adverse effects. Thus, new therapeutic approaches are urgently needed to improve outcomes for patients with immune-mediated disorders. Ideally, this goal could be achieved by more specific delivery of the immunomodulating agent directly to the site(s) of immunopathology, obviating the need for systemic immunosuppression.

As explained above, cells that may have anti-inflammatory potential (such as MSCs) lack the ability to home to sites of inflammation. Furthermore, the biological mechanisms that trigger the anti-inflammatory biology of cells are not fully understood. Thus, there exists a need for compositions and methods for modifying cells to produce therapeutically effective amounts of tissue reparative, immunomodulatory, and anti-inflammatory molecules and to deliver those cells and/or molecules to sites of inflammation in a subject. Moreover, for use of MSCs in cell-based therapy for immunomodulation and/or repair/regeneration of damaged tissues, there is a benefit to have the MSC secretome (i.e., released extracellular vesicles (including all exosomes, membrane particles, and microvesicles) and all secreted soluble molecules) released within the site of tissue injury itself, i.e., within the inflammatory milieu, so that the beneficial anti-inflammatory molecules would be present in highest concentrations precisely where they are most needed. Thus, for most efficient bioactivity, it would be optimal to have MSCs delivered within the parenchyma of affected tissue(s). Once colonized within a desired location, the MSCs could then begin their reparative program that is required to mitigate inflammation and/or achieve pertinent tissue repair/regeneration. Furthermore, there is a need for methods that would achieve a much more efficient delivery of MSCs into the desired anatomic site(s) to lower the cell dose(s) needed for therapeutic benefit(s), obviating the pertinent high-costs of cell production commensurate with administration of MSCs at megadoses. There is also a need for methods that would groom/prime the MSCs to possess heightened bioactivity that would meaningfully lower the amount of administered cells needed for the chosen therapeutic effect(s). Accordingly, there is a need for strategies to optimize tissue delivery of MSCs, and strategies that can, prior to cell administration, prime (via appropriate in vitro methods) the increased production and/or release of the bioactive agents of these cells to be most effective at achieving the intended biologic effect(s). The present disclosure is directed to meeting these and other needs.

### SUMMARY OF THE INVENTION

The present disclosure provides compositions and methods using a novel approach to achieve MSC tissue-reparative effects. As disclosed herein, it has been surprisingly discovered that the inability of MSCs to traffic to inflammatory sites is a significant limitation to achieve potent immunosuppressive effects in vivo. Thus, disclosed herein are compositions and methods to overcome this limitation. The present disclosure provides, inter alia, compositions and methods to modify an MSC's ability to traffic to affected tissues, and to magnify/augment the immunoregulatory/anti-inflammatory/tissue-reparative activity of MSCs prior to their administration, thereby providing a potent cell-based therapy to dampen inflammation and preserve tissue integrity/hasten tissue regeneration. Thus, the present disclosure provides, inter alia, compositions and methods to achieve a cell product, such as MSCs, that are optimally armed (i.e., primed) via in vitro manipulation to boost their immunoregulatory/anti-inflammatory/tissue-preservation/tissue-reparative properties and/or to traffic to inflammatory sites/areas of tissue damage.

The present disclosure also provides, inter alia, compositions and methods to treat immune-mediated diseases. For example, instead of using potentially toxic pharmacologic agents that suppress inflammation-associated upregulated E-selectin expression (e.g., steroids, anti-TNFα agents), endothelial E-selectin display is exploited to enable efficient migration of systemically administered E-selectin ligand-bearing cells. To this end, the present disclosure provides, inter alia, exofucosylated MSCs that are a safe and effective alternative to current pharmacotherapies, ushering a new era of cell-based therapy for a variety of inflammatory disorders. Moreover, the present disclosure provides, inter alia, compositions and methods utilizing the key role for CD44 engagement in triggering MSC immunomodulatory effects, thereby exploiting the interplay between matrix elements and MSCs in maintaining and/or establishing tissue immunohomeostasis.

The present disclosure also provides, inter alia, compositions and methods for treating inflammatory disorders using populations of cells, such as MSCs, that have been modified to produce increased levels of anti-inflammatory and immunomodulatory molecules, such as anti-inflammatory cytokines. As disclosed herein, using a highly reproducible murine model of florid inflammation and immunopathology (splenocyte-enriched full-MHC-mismatched allogeneic hematopoietic stem cell transplantation (allo-HSCT/S) to induce lethal acute graft-versus-host disease (aGvHD)), and by analyzing the impact on tissue immunopathology and host survival of early post-transplant systemic administration of host-type murine adipose-derived MSCs (AdMSCs), either unmodified (“UmAdMSCs”, i.e., HCELL⁻ mAdMSCs) or fucosylated host-type AdMSCs (“FucmAdMSCs”, i.e., HCELL⁻ mAdMSCs), it has been surprisingly found that administration of HCELL⁻ mAdMSCs results in targeted recruitment of mAdMSCs to lesional tissues in aGvHD model, with commensurate dampening of inflammatory infiltrates, reversal of the ratios of serum pro-inflammatory and anti-inflammatory cytokines, and superior survival and deterrence/prevention of immunopathology. Furthermore, it has been surprisingly found using mechanistic studies that ligation of surface CD44 of either murine or human MSCs (for example, via HCELL binding to E-selectin or via CD44 binding to its native ligand HA) potentiates MSC immunomodulation by triggering MSC secretion of multiple immunosuppressive molecules. Thus, the present disclosure provides, inter alia, compositions and methods utilizing the cellular biology of tissue-resident cells, including MSCs as key effectors of immunohomeostasis to promote, e.g., MSC immunoregulatory activity and to prevent and/or reverse immunopathology.

According to some aspects, the present disclosure provides a pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells.

According to some aspects, the present disclosure provides a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells.

According to some aspects, the present disclosure provides a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of CD44⁺ cells.

According to some aspects, the present disclosure provides a method of treating a disease or disorder associated with elevated levels of at least one pro-inflammatory molecule in a subject comprising administering to the subject: (i) a pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; (ii) a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs.

According to some aspects, the present disclosure provides a method of modulating the effects of a cytokine storm in a subject, the method comprising administering to the subject before, during or after onset of the cytokine storm: (i) a pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; (ii) a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs.

According to some aspects, the present disclosure provides a low dose pharmaceutical composition in unit dosage form for intravascular, direct injection, topical or aerosol delivery to a subject comprising: (i) a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; (ii) a conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs.

In some embodiments, CD44 ligand is a naturally occurring ligand. In some embodiments, the CD44 ligand is an artificial ligand. In some embodiments, a naturally occurring glycan modification of CD44 is altered ex vivo to permit and/or promote binding of a ligand to CD44. In some embodiments, the CD44⁺ cells have been modified ex vivo via sialidase to remove terminal sialic acids on CD44 O-glycans or N-glycans and treated with HA. In some embodiments, a glycan decorating the CD44 is altered to promote binding of one or more selectins. In some embodiments, the CD44⁺ cells are treated ex vivo with one or more fucosyltransferases to enforce expression of HCELL and to promote binding to E-selectin or L-selectin.

In some embodiments, a glycosyltransferase is used to install a chemically reactive group, orthogonal functional group, or molecular tag on the CD44, and ligand binding to the chemically reactive group, orthogonal functional group, or molecular tag is effective to promote production of the elevated levels of the one or more anti-inflammatory or immunomodulatory molecules. In some embodiments, the CD44 is ligated with an agent effective to promote the elevated levels of the one or more anti-inflammatory or immunomodulatory molecules. In some embodiments, the CD44⁺ cells have been modified ex vivo to express HCELL and treated with one or more of E-selectin, L-selectin, CSLEX-1 mAbs, and HECA452 mAbs.

In some embodiments, the CD44⁺ cells are mesenchymal stem cells (MSCs), hematopoietic stem cells, tissue stem/progenitor cells, umbilical cord-derived stem cells, stromal vascular fraction, or embryonic stem cells, induced pluripotent stem cells, differentiated progenitors derived from embryonic stem cells or from induced pluripotent stem cells, differentiated progenitors derived from adult stem cells, primary cells isolated from blood or any tissue, a culture-expanded progenitor cell population, a culture-expanded stem cell population, or a culture-expanded primary cell population. In some embodiments, each one or more anti-inflammatory or immunomodulatory molecule is the same or different molecule. In some embodiments, the one or more anti-inflammatory or immunomodulatory molecule comprises IL-10.

According to some aspects, the present disclosure provides a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells.

According to some aspects, the present disclosure provides a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells.

According to some aspects, the present disclosure provides a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells.

According to some aspects, the present disclosure provides a pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells.

In some embodiments, the population of cells are mesenchymal stem cells (MSCs), hematopoietic stem cells, tissue stem/progenitor cells (for example, a neural stem cell, myocyte stem cell or pulmonary stem cell), stromal vascular fraction cells, umbilical cord-derived stem cells, or embryonic stem cells, induced pluripotent stem cells, differentiated progenitors derived from embryonic stem cells or from induced pluripotent stem cells, differentiated progenitors derived from adult stem cells, primary cells isolated from any tissue (e.g., blood, bone marrow, brain, liver, lung, gut, stomach, fat, muscle, testes, uterus, ovary, skin, spleen, eye, endocrine organ and bone), a culture-expanded progenitor cell population, a culture-expanded stem cell population, or a culture-expanded primary cell population. In some embodiments, the population of cells are mesenchymal stem cells. In some embodiments, the population of cells are culture-expanded mesenchymal stem cells. In some embodiments, the population of cells are culture-expanded mammalian adipose-derived mesenchymal stem cells (AdMSCs). In some embodiments, the population of cells are culture-expanded human adipose-derived mesenchymal stem cells (hAdMSCs). In some embodiments, the culture-expanded hAdMSCs are modified ex vivo via treatment with HA.

In some embodiments, the at least one additional anti-inflammatory molecule is selected from TGF-β, IDO, nitric oxide (NO) metabolites, PGE₂ and combinations thereof. In some embodiments, the IL-10 production is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of the cells. In some embodiments, the IL-10 production is elevated at least 3-fold relative to a native population of the cells. In some embodiments, the IL-10 production is elevated at least 10-fold relative to a native population of the cells. In some embodiments, the composition is useful for decreasing plasma levels of at least one pro-inflammatory molecule in a subject when administered to the subject. In some embodiments, the at least one pro-inflammatory molecule comprises a group selected from IFNγ, TNFα, IL-1α, IL-1β, IL-6, IL-12, IL-17 and combinations thereof. In some embodiments, the E-Selectin or L-selectin is an E-Selectin-immunoglobulin or L-selectin-immunoglobulin chimera (E-Ig chimera or L-Ig chimera).

In some embodiments, the pharmaceutical composition is useful for the treatment of a disease associated with one or more of neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, systemic inflammatory response syndrome, acute respiratory disease syndrome secondary to bacteria, fungi or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.). In some embodiments, the pharmaceutical composition is useful for the treatment of a disease associated with a cytokine storm. In some embodiments, the pharmaceutical composition is useful for engendering immunohomeostasis in a subject. In some embodiments, the pharmaceutical composition is useful for the treatment of graft versus host (GvH) disease. In some embodiments, the pharmaceutical composition is useful for the treatment of COVID-19 infection or sequelae of COVID-19 infection (e.g., Kawasaki disease). In some embodiments, the subject is a human.

According to some aspects, the present disclosure provides a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs. According to some aspects, the present disclosure provides a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs. According to some aspects, the present disclosure provides a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule. According to some aspects, the present disclosure provides a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs. According to some aspects, the present disclosure provides a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs. According to some aspects, the present disclosure provides a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.

In some embodiments, the population of cells is culture-expanded. In some embodiments, the HA-primed cells are exofucosylated to enforce hematopoietic cell E-Selectin/L-Selectin Ligand (HCELL) prior to administration to a subject. In some embodiments, the E-Selectin or L-selectin-primed cells are exofucosylated a second time in vitro to enforce hematopoietic cell E-Selectin/L-Selectin Ligand (HCELL) prior to administration to a subject. In some embodiments, the E-Selectin or L-selectin is an E-Selectin-immunoglobulin or L-selectin-immunoglobulin chimera (E-Ig chimera or L-Ig chimera). In some embodiments, the IL-10 production is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of the cells. In some embodiments, the IL-10 production is elevated at least 3-fold relative to a native population of the cells. In some embodiments, the IL-10 production is elevated at least 10-fold relative to a native population of the cells.

In some embodiments, the population is useful for decreasing plasma levels of at least one pro-inflammatory molecule when administered to a subject. In some embodiments, the at least one pro-inflammatory molecule comprises a group selected from IFNγ, TNFα, IL-1α, IL-1β, IL-6, IL-12, IL-17 and combinations thereof. In some embodiments, the at least one additional anti-inflammatory molecule is selected from TGF-β, IDO, nitric oxide (NO) metabolites, PGE₂ and combinations thereof. In some embodiments, the population is useful for the treatment of a disease associated with one or more of neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, systemic inflammatory response syndrome, acute respiratory disease syndrome secondary to bacteria, fungi or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.). In some embodiments, the population is useful for the treatment of a disease associated with a cytokine storm. In some embodiments, the population is useful for engendering immunohomeostasis in a subject. In some embodiments, the population is useful for the treatment of graft versus host (GvH) disease. In some embodiments, the population is useful for the treatment of COVID-19 infection. In some embodiments, the subject is a human.

According to some aspects, the present disclosure provides a unit dose of the population according to any of the aspects or embodiments disclosed herein comprising an effective amount of the primed cells. In some embodiments, the effective amount is selected from at least about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg. In some embodiments, the effective amount is less than about one million primed cells/kg. In some embodiments, the effective amount is between about 50,000 to about 950,000 primed cells/kg.

According to some aspects, the present disclosure provides a method of treating a disease or disorder associated with elevated levels of at least one pro-inflammatory molecule in a subject comprising administering to the subject: (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells; (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells; (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells; (iv) a pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells; (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs; (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs; (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule; (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs; (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.

In some embodiments, the disease or disorder associated with elevated levels of at least one pro-inflammatory molecule is selected from neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, systemic inflammatory response syndrome, acute respiratory disease syndrome secondary to bacteria, fung or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.). In some embodiments, the disease or disorder associated with elevated levels of at least one pro-inflammatory molecule is a cytokine storm. In some embodiments, the disease or disorder associated with elevated levels of at least one pro-inflammatory molecule is graft versus host (GvH) disease. In some embodiments, the disease or disorder is associated with elevated levels of at least one pro-inflammatory molecule (e.g., IL-6). In some embodiments, the E-selectin or L-selectin-primed cells are further exofucosylated prior to administration to the subject. In some embodiments, the HA-primed cells are exofucosylated prior to administration to the subject.

According to some aspects, the present disclosure provides a method of modulating the effects of a cytokine storm in a subject, the method comprising administering to the subject before, during or after onset of the cytokine storm: (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells; (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells; (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells; (iv) a pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells; (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs; (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs; (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule; (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs; (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.

In some embodiments, the IL-10 production is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of cells. In some embodiments, the IL-10 production is elevated at least 3-fold relative to a native population of cells. In some embodiments, the IL-10 production is elevated at least 10-fold relative to a native population of cells. In some embodiments, the at least one pro-inflammatory molecule comprises a group selected from IFNγ, TNFα, IL-1α, IL-1β, IL-6, IL-12, IL-17 and combinations thereof. In some embodiments, the at least one pro-inflammatory molecule is the cytokine IL-6. In some embodiments, the at least one additional anti-inflammatory molecule is selected from TGF-β, IDO, nitric oxide (NO) metabolites, PGE₂ and combinations thereof. In some embodiments, the increase in IL-10 production and decrease in plasma levels of at least one pro-inflammatory molecule is observed for a prolonged period of time. In some embodiments, the prolonged period of time is for at least 5 days, 10 days, at least 20 days or at least 30 days. In some embodiments, the pharmaceutical composition or population of cells is administered to the subject topically, intravascularly, by direct injection, or as an aerosol.

According to some aspects, the present disclosure provides the method according to any aspect or embodiment disclosed herein, further comprising administering the pharmaceutical composition or population of cells as an adjuvant to a primary immunotherapy. In some embodiments, the administration step comprises delivering to the subject about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg. In some embodiments, the administration step comprises delivering to the subject less than about one million primed cells/kg. In some embodiments, the administration step comprises delivering to the subject between about 50,000 to about 950,000 primed cells/kg. In some embodiments, the subject is a human. In some embodiments, the E-selectin- or L-selectin-primed cells are further exofucosylated prior to administration to the subject. In some embodiments, the HA-primed cells are exofucosylated prior to administration to a subject.

According to some aspects, the present disclosure provides a low dose pharmaceutical composition in unit dosage form for intravascular (e.g., intravenous), direct injection, topical or aerosol delivery to a subject comprising: (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells; (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells; (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻CD44⁺/PSGL⁻ cells; (iv) a pharmaceutical composition comprising conditioned media obtained from a population of CD34⁻/CD44⁺/PSGL⁻CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻CD44⁺/PSGL⁻ cells; (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs; (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs; (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule; (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs; (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.

In some embodiments, the low dose pharmaceutical composition comprises about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg. In some embodiments, the low dose pharmaceutical composition comprises less than about one million primed cells. In some embodiments, the low dose pharmaceutical composition comprises between about 50,000 to about 950,000 primed cells/kg. In some embodiments, the E-selectin or L-selectin-primed cells are further exofucosylated prior to administration to a subject. In some embodiments, the HA-primed cells are exofucosylated prior to administration to a subject.

According to some aspects, the present disclosure provides a method of producing a pharmaceutical composition for treating a disease or disorder associated with elevated levels of at least one pro-inflammatory molecule in a subject comprising: ex vivo exofucosylating a stem cell and culturing the stem cell under conditions sufficient to enforce hematopoietic cell E-Selectin/L-Selectin Ligand (HCELL) expression on a surface of the stem cell at levels above what is natively present on the stem cell and treating the HCELL⁺ stem cell with E-selectin or L-selectin to prime the HCELL⁺ stem cells to (a) produce elevated levels of interleukin-10 (IL-10) and at least one additional anti-inflammatory molecule relative to a native population of the stem cells and/or to (b) induce a decrease in the plasma levels of at least one pro-inflammatory molecule when administered to a subject.

According to some aspects, the present disclosure provides a method of producing a pharmaceutical composition for treating a disease or disorder associated with elevated levels of at least one pro-inflammatory molecule in a subject comprising: ex vivo ligating CD44 on a surface of a stem cell with hyaluronic acid (HA) and culturing the stem cell under conditions sufficient to prime the stem cell to (a) produce elevated levels of interleukin-10 (IL-10) and at least one additional anti-inflammatory molecule relative to a native population of the stem cells and/or to (b) induce a decrease in the plasma levels of at least one pro-inflammatory molecule when administered to a subject.

In some embodiments, the method further comprises culture expanding the stem cell prior to exofucosylation. In some embodiments, the method further comprises culture expanding the stem cell prior to ligation of CD44 with HA. In some embodiments, the method further comprises preparing a unit dosage form comprising about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg. In some embodiments, the method further comprises preparing a unit dosage form comprising less than about one million primed cells. In some embodiments, the method further comprises preparing a unit dosage form comprising between about 50,000 to about 950,000 primed cells/kg. In some embodiments, the method further comprises exofucosylating the E-selectin or L-selectin-primed cells prior to administration to a subject. In some embodiments, the culture conditions comprise incubating the HA with the cells for up to about 72 hours. In some embodiments, the culture conditions comprise incubating the HA with the cells for at least about 24 hours. In some embodiments, the culture conditions comprise incubating the HA with the cells for between about 24-72 hours. In some embodiments, the method further comprises exofucosylating the HA-primed cells prior to administration to a subject. In some embodiments, the stem cells are harvested from the subject prior to the ex vivo modification. In some embodiments, the stem cells are harvested from a compatible donor prior to the ex vivo modification. In some embodiments, the exofucosylation is carried out with a glycosyltransferase together with donor nucleotide sugar. In some embodiments, the glycosyltransferase is an alpha 1,3-fucosyltransferase. In some embodiments, the alpha 1,3-fucosyltransferase is alpha 1,3-fucosyltransferase FTIII, FTIV, FTV, FTVI, FTVII, and combinations thereof. In some embodiments, the alpha 1,3-fucosyltransferase is human FTVII. In some embodiments, the stem cell is selected from the group consisting of embryonic stem cells, adult stem cells, hematopoietic stem cells and induced pluripotent stem cells (iPSCs). In some embodiments, the stem cell is an MSC. In some embodiments, the stem cell is an AdMSC. In some embodiments, the stem cell is a hAdMSC. In some embodiments, the method further comprises harvesting conditioned media from the modified cells.

According to some aspects, the present disclosure provides a pharmaceutical composition produced by any of the methods disclosed herein.

According to some aspects, the present disclosure provides a method of treating a disease or disorder associated with elevated levels of at least one pro-inflammatory molecule in a subject comprising: (i) preparing a pharmaceutical composition according to any method disclosed herein; and (ii) administering the pharmaceutical composition from step (i) to the subject. In some embodiments, the administering step comprises intravascular, direct injection, topical or aerosol delivery to the subject. In some embodiments, the pharmaceutical composition is administered to the subject in a low dose unit dosage form. In some embodiments, the low dose comprises about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg. In some embodiments, the low dose comprises less than about one million primed cells. In some embodiments, the low dose comprises between about 50,000 to about 950,000 primed cells/kg.

According to some aspects, the present disclosure provides a method of selecting a population of CD44⁺ cells that are effective for treatment of inflammatory disorders comprising the steps of: (i) modifying ex vivo the population of CD44⁺ cells via exofucosylation to enforce hematopoietic cell E-Selectin/L-Selectin Ligand (HCELL) expression and treating the ex vivo modified cells with E-selectin or L-selectin; or modifying ex vivo the population of CD44⁺ cells via treatment with hyaluronic acid (HA); (ii) detecting in the modified population of CD44⁺ cells of step (1) production of an anti-inflammatory or immunomodulatory molecule; and (iii) selecting the modified cells that produce elevated levels of the anti-inflammatory or immunomodulatory molecule relative to a native population of CD44⁺ cells for use in the treatment of inflammatory disorders.

According to some aspects, the present disclosure provides any method, composition or population disclosed herein comprising enhancing CD44-HA, HCELL-HA, HCELL-E-Selectin, or HCELL-L-selectin binding with an agent. In some embodiments, the agent is an antibody to CD44 or antigen binding fragment thereof that cross-links CD44 or that functions to upregulate the ability of CD44⁺ cells to bind HA or that functions to enhance HCELL binding to E-Selectin or L-selectin.

According to some aspects, the present disclosure provides a population of hAdMSCs, in isolated form, that express hematopoietic cell E-Selectin/L-Selectin Ligand (HCELL) at a level that exceeds the level of HCELL expression by native hAdMSCs as assessed by Western blot using monoclonal antibody HECA-452 and express interleukin-10 (IL-10) and at least one additional anti-inflammatory molecule at a level that exceeds the level of expression of each such molecule by native hAdMSCs as assessed using culture supernatant by ELISA or a modified Griess reagent in the case of nitric oxide (NO) metabolites.

According to some aspects, the present disclosure provides a pharmaceutical composition for administration to a subject comprising a population of hAdMSCs that express (i) hematopoietic cell E-Selectin/L-Selectin Ligand (HCELL) at a level that exceeds the level of HCELL expression by native hAdMSCs as assessed by Western blot using monoclonal antibody HECA-452 and (ii) interleukin-10 (IL-10) that exceeds the level of expression of IL-10 by native hAdMSCs as assessed using culture supernatant by ELISA. In some embodiments, the population of hAdMSCs express at least one additional anti-inflammatory molecule that exceeds the level of production of such anti-inflammatory molecule relative to native hAdMSCs as assessed using culture supernatant by ELISA or a modified Griess reagent in the case of nitric oxide (NO) metabolites. In some embodiments, the pharmaceutical composition is in dosage unit form comprising the population of hAdMSCs and a pharmaceutically acceptable excipient, wherein the population of hAdMSCs contained within the dosage unit does not exceed one million hAdMSC cells.

According to some aspects, the present disclosure provides a method of treating a disease or disorder associated with elevated levels of at least one pro-inflammatory molecule in a subject comprising administering to the subject the population of hAdMSCs as disclosed herein or the pharmaceutical composition as disclosed herein. In some embodiments, the administration step is selected from intravascular, direct injection, topical or aerosol delivery to the subject.

According to some aspects, the present disclosure provides the use of the population of hAdMSCs as disclosed herein or the pharmaceutical composition as disclosed herein for use in the treatment of a disease or disorder associated with elevated levels of at least one pro-inflammatory molecule.

According to some aspects, the present disclosure provides a method as disclosed herein wherein the pharmaceutical composition or population is effective to increase the local level of one or more anti-inflammatory molecules upon local administration to a lesional site in a subject by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to the local level of the one or more inflammatory molecules before local administration.

According to some aspects, the present disclosure provides a method as disclosed herein, wherein the pharmaceutical composition or population is effective to decrease the local anatomic tissue/fluid level of one or more pro-inflammatory molecules upon localized (e.g., by direct injection) administration to a lesional site in a subject by at least 5%, 10%, 25%, 50%, 80% or 90% relative to the local level of the one or more inflammatory molecules before the local administration.

According to some aspects, the present disclosure provides compositions and methods as disclosed herein, wherein the conditioned media comprises one or more of microvesicles and exosomes. According to some embodiments, the methods disclosed herein further comprise isolating the one or more of microvesicles and exosomes from the conditioned media.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1A-1F. E-selectin expression is upregulated in intestine and liver microvessels in aGVHD. (FIGS. 1A and 1B): Immunohistochemical staining of sequential sections of C57BL/6 intestines (FIG. 1A) or livers (FIG. 1B) showing colocalization of E-selectin and endothelial marker CD31 (delimited by arrowheads) in mice with ongoing aGvHD compared to bone marrow transplanted mice (no aGvHD) (magnification ×400) (n=5 mice per group). (FIG. 1C-1E): Flow cytometry histograms of mAdMSCs stained with mAb HECA452 mAb and mouse E-selectin-Ig chimera (mE-Ig). For each panel, specific antibody staining is shown as shaded histogram and staining of corresponding control is shown as empty histogram. (FIG. 1C) Native mAdMSCs are devoid of reactivity with HECA452 and with mE-Ig. (FIG. 1D) Following FTVII treatment, FucmAdMSCs display substantial staining with mAb HECA452 and Ca²⁺-dependent binding of mE-Ig chimera (dark shaded histograms) compared to control isotype or EDTA-buffer (w/o Ca²⁺), respectively (empty histograms). (FIG. 1E) Digestion of FTVII-treated AdMSCs with proteinase K and bromelain significantly reduces Ca²⁺-dependent mE-Ig binding but not HECA452 staining (light grey histograms). (FIG. 1F) Whole lysates of FucmAdMSCs or UmAdMSCs were subjected to western blot to analyze E-selectin binding (left panel). Thereafter, membranes were stripped and analyzed with anti-CD44 mAb for protein loading control (right panel). As shown, exofucosylation (+) of mAdMSCs engenders expression of HCELL (CD44 band at ˜100 kDa). Results are representative of n=5 separate experiments.

FIG. 2A-2E. FTVII treatment significantly increases mAdMSC colonization within aGvHD-target organs. (FIG. 2A) Schematic of the experimental protocol for mAdMSCs administration after allo-HSCT/S. (FIG. 2B, FIG. 2D): Sections of small intestines (FIG. 2B) or livers (FIG. 2D) are shown from recipient C57BL/6 mice with ongoing aGvHD that were administered GFP-transgenic-mAdMSCs either unmodified (UmAdMSCs) or FTVII-modified (FucmAdMSCs); sections were stained for expression of GFP by anti-GFP ABC colorimetric immunohistochemistry. Representative images of small intestines and livers from both groups of treated animals at day 10, 20 and 30 post-transplant show infiltrating GFP⁺ mAdMSCs (magnification ×200). Arrowheads indicate infiltrating GFP⁺ mAdMSCs in intestinal lamina propria or in hepatic portal area, respectively. (FIG. 2C, FIG. 2E): Bar graphs represent absolute GFP⁺ mAdMSC infiltrate counts in small intestines (FIG. 2C) or livers (FIG. 2E) as mean±SD from counts relative to 1 mm² (n=5 mice per group). GFP⁺ mAdMSCs infiltration was significantly increased in recipients of FucmAdMSCs compared to that of UmAdMSCs, **p<0.01 or ***p<0.001, respectively (analyzed by Student's paired t-Test).

FIG. 3A-3D. Allo-HSCT/S recipients administered HCELL⁺ mAdMSCs have improved survival, improved aGVHD scores, and significantly lower CD3+ lymphocyte infiltrates in liver and gut. C57BL/6 recipient mice were transplanted intravenously via tail vein with 1×10⁷ bone marrow cells (“BM only transplant”) or with 1×10⁷ bone marrow cells enriched with 1.5×10⁷ donor splenocytes to induce aGvHD (“Allo-HSCT/S”). On day +0, +7 and +14 allo-HSCT/S recipient mice received an intravenous infusion of 5×10⁴ mAdMSCs, either unmodified (“UmAdMSCs” mice) or FTVII-modified mAdMSCs (“FucmAdMSCs” mice). At the same time periods, another group of allo-HSCT/S mice received an equal volume of saline solution (“Untreated” mice). (FIG. 3A) Kaplan-Meier survival curves of recipient C57BL/6 of the different groups are shown (n=16 animals per group). Survival in all-HSCT/S mice administered HCELL⁺ mAdMSCs (“FucmAdMSCs”) was significantly higher compared to the allo-HSCT/S “Untreated” (i.e., no administration of mAdMSCs) group (***p<0.001) or compared to allo-HSCT/S mice receiving UmAdMSCs (^{Δ}p<0.05) (analyzed by Log-rank (Mantel-Cox) test). (FIG. 3B) Clinical aGvHD score for each experimental group was assessed using a composite scoring system consisting of 5 clinical individual scores (weight loss, posture, activity, fur texture, and skin integrity (maximum index=10)). Clinical aGvHD score in allo-HSCT/S mice administered HCELL⁺ mAdMSCs (“FucmAdMSCs”) was significantly reduced compared to “Untreated” group of animals (***p<0.001) or compared to mice administered UmAdMSCs (^{ΔΔΔ}p<0.001) (analyzed by one-way ANOVA with Bonferroni's multiple-comparisons test). (FIG. 3C) Tissues from animals with ongoing aGvHD, either Untreated or treated with either type of mAdMSCs, were isolated after 10, 20 or 30 days after allo-HSCT/S. CD3⁺ T cell inflammatory infiltrate was then detected by standard anti-CD3 ABC colorimetric immunohistochemistry. Representative images of CD3⁺ T cell infiltrates within liver and gut from the different mouse groups are shown (magnification ×200). (FIG. 3D) Bar graphs depict absolute T cells counts (CD3+ cells), presented as mean±SD per high-power field from counts relative to 10 high-power fields (magnification ×200) (n=5 mice per group). Cross symbols indicate that Untreated animals (i.e., mice not receiving mAdMSCs) did not survive at these time points of analysis. Compared to Untreated mice, mice receiving UmAdMSCs and FucmAdMSCs had significantly less T cell infiltrates, with lowest infiltrates in those mice receiving FucmAdMSCs (**p<0.01 or ***p<0.001, analyzed by one-way ANOVA with Bonferroni's multiple-comparisons test).

FIG. 4A-4B. Temporal evolution of polymorphonuclear neutrophil (PMN) infiltrates in aGvHD-target organs in mice receiving either UmAdMSCs or FucmAdMSCs. Tissues from animals with aGvHD, either Untreated or treated with UmAdMSCs/FucmAdMSCs, were isolated after 10, 20 or 30 days post-transplantation. PMNs were identified in tissue sections on basis of characteristic morphologic appearance of lobulated nuclei. (FIG. 4A) Representative images of PMN infiltrates within liver and gut affected by aGvHD (H&E stain, magnification ×400). (FIG. 4B) Absolute PMN counts are presented as mean±SD per high-power field from counts relative to 10 high-power fields (magnification ×400) (n=5 mice per group). Cross symbols indicate that Untreated animals did not survive at these time points of analysis. As shown by asterisks, statistically significant differences in the extent of PMN infiltrates were observed among mice that received UmAdMSCs versus those that received FucmAdMSCs, *p<0.05 or **p<0.01, respectively (analyzed by one-way ANOVA with Bonferroni's multiple-comparisons test).

FIG. 5. Intravenous infusion of FTVII-treated AdMSCs alters the systemic profile of secreted pro-inflammatory and anti-inflammatory cytokines on mice with aGvHD. Plasma concentrations of the pro-inflammatory factors IFNγ, TNFα, IL-1β, IL-6, IL-12, and IL-17, and anti-inflammatory molecules TGFβ, IL-10 and PGE₂ were measured on days +10, +20 or +30 post-transplantation by ELISA. Data are presented as mean±SD of n=5 animals per group. Cross symbols indicate that Untreated animals did not survive at these time points of analysis. As shown in the panels, plasma levels of all the inflammatory cytokines tested were significantly decreased with administration of FucmAdMSCs (*p<0.05, **p<0.01, ***p<0.001), whereas levels of anti-inflammatory cytokines TGFβ and IL-10 were markedly increased (^{Δ}p<0.05, ^{ΔΔ}p<0.01, ^{ΔΔΔ}p<0.001) (analyzed by one-way ANOVA with Bonferroni's multiple-comparisons test).

FIG. 6A-6G. Effects of HCELL/CD44 engagement on immunomodulatory properties of mAdMSCs. (FIG. 6A, FIG. 6B): (FIG. 6A) Splenocytes (Spl) from C57BL/6 (syngeneic context) or from (FIG. 6B) BALB/C mice (allogeneic context) were stimulated with concanavalin A (ConA) in the presence of different ratios of unmodified (UmAdMSCs) or FTVII-modified AdMSCs (FucmAdMSCs) from C57BL/6 mice. Mitogen-induced proliferation of responder splenocytes was measured by incorporation of BrdU. In presence of mAdMSCs, proliferation of responder splenocytes was significantly inhibited (**p<0.01 or ***p<0.001). (FIG. 6C-FIG. 6F) BALB/C splenocytes were stimulated with ConA in the presence of UmAdMSCs, FucmAdMSCs or sialidase-treated UmAdMSCs (sialUmAdMSCs) all derived from derived from C57BL/6 mice) at an MSC:splenocyte ratio of 1:50 (allogeneic context) that were previously cultured for: (FIG. 6C) 24 h with E-selectin (mE-Ig) (left) or hyaluronic acid (HA) (right), or previously cultured for 72 h with (FIG. 6D) mE-Ig, (FIG. 6E) HA or (FIG. 6F) both ligands, and then maintained in wells containing mE-Ig, HA, or both, respectively. As controls, E-selectin adherence of FucmAdMSCs was abrogated by sialidase treatment of the mAdMSCs (sialFucmAdMSCs), or by blocking adherence of mAdMSC to HA by culturing in presence of a blocking anti-CD44 antibody. Responder splenocyte proliferation was significantly inhibited (*p<0.05, **p<0.01 or ***p<0.001), respectively. (FIG. 6G) Inhibition of splenocyte proliferation in presence of conditioned media obtained from HA- or mE-Ig-ligated UmAdMSCs, FucmAdMSCs or sialFucmAdMSCs (right) was analyzed compared to levels obtained in the continuous presence of the same type of mAdMSCs (left). As shown in panel at right, proliferation was significantly inhibited in presence of supernatant alone (***p<0.001). Data were analyzed by one-way ANOVA with Bonferroni's multiple-comparisons test.

FIG. 7A-7D. Levels of TGFβ, IDO and of NO metabolites in supernatants of mAdMSCs after HCELL or CD44 ligation. UmAdMSCs or FucmAdMSCs were cultured in the presence of different concentrations of E-selectin (mE-Ig) or hyaluronic acid (HA) at 37° C. for 24 h, and culture supernatants were then collected. (FIG. 7A-7C): Levels of anti-inflammatory molecules (FIG. 7A) TGFβ, (FIG. 7B) IDO and (FIG. 7C) NO metabolites (e.g., NO₂⁻/NO₃⁻) were measured by ELISA techniques. Levels of each molecule was significantly increased by ligation of CD44 or of HCELL as shown (*p<0.05 or **p<0.01); treatment of FucmAdMSCs with sialidase (sialFucmAdMSCs”) abrogated HCELL ligation with mE-Ig, with commensurate decreased levels of anti-inflammatory molecules compared to levels found in FucmAdMSCs cultures exposed to E-selectin (^{Δ}p<0.05 or ^{ΔΔ}p<0.01). (FIG. 7D) Inhibitory agents to each molecule were introduced into co-cultures of BALB/c splenocytes and C57BL/6 UmAdMSCs, FucmAdMSCs or sialFucmAdMSCs (MSC:splenocyte ratio of 1:20) previously adhered to HA or mE-Ig for 72 h. Mitogen-induced splenocyte proliferation was calculated by subtracting the level of splenocyte basal proliferation in the absence of ConA. Addition of SB-431542 (TGFβ inhibitor), 1-methyl-DL-tryptophan (1-MT) (IDO inhibitor) or N^{G}-monomethyl-L-arginine (L-NMMA) (iNOS inhibitor) led to significant increases in proliferation of responder splenocytes (**p<0.01 or ***p<0.001). Splenocyte proliferation was significantly decreased compared to same conditions in absence of HA or E-selectin, ^{□□}p<0.01, or increased compared to same conditions using FucmAdMSCs, ^{##}p<0.01, respectively. All data are presented as the mean±SD of n=3 separate experiments and analyzed by one-way ANOVA with Bonferroni's multiple-comparisons test.

FIG. 8A-8B. Effects of HCELL/CD44 engagement on immunomodulatory properties of human MSCs. (FIG. 8A and FIG. 8B) Human MSCs derived from adipose tissue (hAdMSCs) or bone marrow (hBMMSCs) were fucosylated (“Fuc”: FuchAdMSCs or FuchBMMSCs) or buffer-treated (unmodified (“U”): UhAdMSCs or UhBMMSCs) and cultured in presence of different concentrations of E-selectin (mE-Ig) or hyaluronic acid (HA) for 3 days at 37° C. Thereafter, culture supernatants were harvested and analyzed by ELISA for (FIG. 8A) levels of anti-inflammatory molecules interleukin-10 (IL-10) and TGFβ, or (FIG. 8B) IDO and NO metabolites (e.g., NO²⁻/NO³⁻). Cells cultured in the absence of HA or in presence of control IgG served as negative controls. Also, as controls to assess specificity of E-selectin binding, FuchAdMSCs or FuchBMMSCs were treated with sialidase (“sialFuchAdMSCs” or “sialFuchBMMSCs”) to cleave terminal sialic acid from sLe^{x} (thereby abrogating binding to E-selectin). As shown in panels, levels of analyzed immunomodulatory molecules significantly increased following hMSC co-incubation with either E-selectin or HA (**p<0.01 or ***p<0.001); levels of immunomodulatory molecules did not rise in sialFuchMSCs co-incubated with E-selectin (compared to FuchMSCs, ^{ΔΔ}p<0.01 or ^{ΔΔΔ}p<0.001, respectively). Overarching bar lines reflect comparisons of immunomodulatory molecule levels between supernatants of hAdMSCs and of hBMMSCs following co-incubation with HA or E-selectin: notably, supernatant levels of all analyzed immunomodulatory molecules were significantly higher (differences of ^{#}p<0.05, ^{##}p<0.01, or ^{###}p<0.001, as shown) in cultures of adipose-derived hMSCs compared to those of bone marrow-derived hMSCs, most conspicuously for IL-10 (###p<0.001). Data are presented as the mean±SD of n=3 separate experiments and analyzed by one-way ANOVA with Bonferroni's multiple-comparisons test.

FIG. 9A-9B. Murine AdMSCs express typical MSC immunophenotype and multipotent differentiation ability. (FIG. 9A) mAdMSCs expressed characteristic MSCs markers such as CD73, CD105, CD90, CD44, CD29, Sca-1, CD166 and CD106, whereas expression of CD34, CD45, CXCR4, c-Kit, CD80 and CD86 were low or negative. Specific antibodies staining (shaded histograms) were compared with their corresponding control isotypes (light grey histograms). The figure shows representative flow cytometry analyses obtained from n=3 separate experiments. (FIG. 9B) mAdMSCs were cultured to test their ability to differentiate in vitro to mesodermal lineages. Adipogenic differentiation was assessed by staining of neutral lipid vacuoles with Oil Red O and by analysis of expression of the adipocyte marker FABP4 by immunofluorescence staining. Differentiation of mAdMSCs into osteoblast was evaluated by the presence of calcium depositions in the cultures by Alizarin Red staining and by detection of alkaline phosphatase activity by staining with BCIP-NBT. Chondrogenic differentiation was analyzed by detection of mucopolysaccarides and glycosaminoglycans by staining with Alcian Blue and by expression of collagen II by immunofluorescence techniques. Images are representative of n=3 separate experiments of differentiation cultures (with triplicates for each condition). Scale bar: 200 mm.

FIG. 10A-10B. Culture supernatant levels of IL-10 and PGE2 in mAdMSCs after HCELL or CD44 ligation. UmAdMSCs or FucmAdMSCs were cultured in the presence of different concentrations of murine E-selectin (mE-Ig) or hyaluronic acid (HA) at 37° C. for 24 h, and culture supernatants were collected. (FIG. 10A) Interleukin-10 (IL-10) or (FIG. 10B) prostaglandin E2 (PGE2) levels were measured by ELISA. Cells cultured in the absence of HA or in presence of control IgG served as negative controls. Also, as controls to assess specificity of E-selectin binding, FucmAdMSCs were treated with sialidase (sialFucmAdMSCs) to cleave terminal sialic acid from sLex. Concentrations levels of both factors are presented as mean±SD for n=3 separate experiments, each performed in triplicates and statistically analyzed by one-way ANOVA with Bonferroni's multiple-comparisons test.

FIG. 11. Ligation of mAdMSC CD44 or HCELL stimulates production of TGFβ, IDO and NO. Co-cultivation of BALB/c splenocytes and C57BL/6 UmAdMSCs, FucmAdMSCs or sialFucmAdMSCs (MSC:splenocyte ratio of 1:10) was performed in presence of concanavalin A (ConA) with or without pre-incubation with hyaluronic acid (HA) or murine E-selectin (mE-Ig). Mitogen-induced splenocyte proliferation was calculated by subtracting the level of splenocyte basal proliferation in the absence of ConA. Addition of SB-431542 (TGFβinhibitor), 1-methyl-DL-tryptophan (1-MT) (IDO inhibitor) or NG-monomethyl-L-arginine (L-NMMA) (iNOS inhibitor) significantly increased the proliferation of responder splenocytes compared to controls, **p<0.01 or ***p<0.001, respectively. All data are presented as the mean±SD of n=3 separate experiments and statistically analyzed by one-way ANOVA with Bonferroni's multiple-comparisons test.

### DETAILED DESCRIPTION

### Ligands of CD44

According to some aspects, the present disclosure provides compositions and methods directed to one or more CD44⁺ cells that have been modified ex vivo to increase production of one or more anti-inflammatory or immunomodulatory molecules via binding of CD44 with a ligand. As used herein, the term “ligand” and grammatical variation thereof means a natural or artificial molecule(s) which bind to CD44 directly or indirectly, and that is effective to promote production of anti-inflammatory or immunomodulatory molecules when ligated to the CD44 present on a cell.

In some embodiments, the CD44 is ligated ex vivo with a molecule that is effective to promote production of anti-inflammatory or immunomodulatory molecules by a cell. In some embodiments, CD44 ligands include naturally occurring ligands (such as an extracellular matrix component) or artificial ligands that are effective to promote production of anti-inflammatory or immunomodulatory molecules by a cell. In some embodiments, CD44 ligands that are effective to promote production of anti-inflammatory or immunomodulatory molecules by a cell include, but are not limited to, hyaluronic acid (HA), osteopontin (OPN), collagens (e.g., Type I and VI), serglycins, galectins (e.g. gal-8), Siglecs, matrix metalloproteinases (MMPs), ARHGEF1, Ezrin (via PIP2), epidermal growth factor receptor (Hyaluronan-dependent), fibrin and fibrinogen, fibronectin, FYN, Lck, selectins, and Src. In some embodiments, the CD44 ligands that are effective to promote production of anti-inflammatory and/or immunomodulatory molecules by a cell are selectins. In some embodiments, the CD44 ligands that are effective to promote production of anti-inflammatory and/or immunomodulatory molecules by a cell include, but are not limited to, E-selectin, L-selectin, and P-selectin.

In some embodiments, the CD44 ligand that is effective to promote production of anti-inflammatory and/or immunomodulatory molecules by a cell is hyaluronic acid (HA). In some embodiments, the HA is a high molecular weight HA (HW HA) that exhibits pro-inflammatory effects in vivo, such as HA with a molecular weight of at least 100,000 daltons.

In some embodiments, the CD44 is ligated with a molecule with a specific binding affinity for CD44, such as for example, an antibody or antigen binding fragment thereof derived from any animal source, including, but not limited to monoclonal antibodies, polyclonal antibodies, phagemids, aptamers, Camel Ig (a camelid antibody (VHH)), Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody). In some embodiments, the CD44 is ligated with a monoclonal or polyclonal antibody effective to initiate or enhance production of anti-inflammatory cytokines in the CD44⁺ cells.

In some embodiments, the CD44 is ligated with a ligand that is effective to enhance the binding of HA to the CD44. In some embodiments, the CD44 is ligated with a monoclonal or polyclonal antibody that is effective to enhance the binding of HA to the CD44. Examples of antibodies that enhance the binding of HA to CD44 include, but are not limited to, IRAWB14 antibody (see, e.g, Zheng, Z., et al., Monoclonal Antibodies to CD44 and Their Influence on Hyaluronan Recognition, The Journal of Cell Biology, Vol. 130, No. 2, 485-495). In some embodiments, the cell bearing CD44 is activated with a small molecule that is effective in enhancing the capacity of CD44 to bind to HA. Examples of small molecules that enhance CD44 binding to HA include, but are not limited to, phorbol myristate acetate (PMA) (see, e.g., Sionov, R. V., et al., Cell Adhes Commun. 1998; 6(6):503-23).

In some embodiments, the CD44 ligand may be a modified ligand. In some embodiments, the CD44 ligand may be a hybrid of, or conjugated to, one or more other molecules. For example, in some embodiments, an E-selectin may be an E-selectin or L-selectin chimera with IgG (i.e., an E-Ig chimera or L-Ig chimera). Such chimeras are readily synthesized by those of skill in the art (Baldo, 2015)) or may be obtained from commercial sources (e.g., R&D systems).

In some embodiments, the cells are treated with the CD44 ligand in amounts and for durations effective to promote production of anti-inflammatory and/or immunomodulatory molecules in the CD44⁺ cells. In some embodiments, the cells are treated for at least 30 minutes, at least 1 hour, at least 2 hours, at least 4 hours, at least 8 hours, at least 12 hours, at least 24 hours, at least 36 hours, or at least 48 hours. In some embodiments, the cells are treated with the CD44 ligand for between about 30 minutes and about 48 hours, such as between about 30 minutes and about 2 hours, between about 30 minutes and 90 minutes, and between about 30 minutes and one hour. In some embodiments, the CD44 is ligated ex vivo with HA for a period of time sufficient to prime the CD44⁺ cells to initiate or enhance production of anti-inflammatory cytokines. Modifying CD44

In some embodiments, the CD44 is altered prior to, or concurrent with, ligation to promote interaction with a ligand. For example, the interactions of CD44 with ligands may be regulated via certain naturally occurring modifications to the intracellular or extracellular regions of CD44. Those naturally occurring modifications may block or reduce the ability of CD44 to bind to a ligand. In some embodiments, the naturally occurring modifications of CD44 are altered ex vivo to permit and/or promote binding of a ligand to CD44. For example, in some embodiments, terminal sialic acids on CD44 O-glycans or N-glycans, which are known to block HA binding, are removed by treatment with one or more sialidases, prior to or concurrent with ligation of HA.

In some embodiments, the glycans decorating CD44 are altered to promote binding of one or more ligands, such as selectins. In some embodiments, CD44 expressing cells are treated ex vivo with one or more glycosidases and/or glycosyltransferases to construct on the CD44 a glycan structure that is effective for ligand binding. In some embodiments, CD44 expressing cells are treated ex vivo with one or more fucosyltransferases, for example, to enforce expression of HCELL, which binds to E-selectin and L-selectin. In some embodiments, a glycosyltransferase is used to install a chemically reactive group, orthogonal functional group, or molecular tag. For example, in some embodiments, addition of a donor GDP-fucose wherein the fucose has been modified by methods known in the art with a chemically reactive group, or orthogonal functional group, or molecular tag (e.g., biotinylated GDP-fucose, azido-GDP-fucose, etc.) thereby allowing for subsequent linkage of other molecules onto the installed fucose within cell surface lactosaminyl glycans (examples of this approach include, but are not limited to, use of biotinylated GDP-fucose with subsequent complexing using streptavidin-conjugated molecules (see, e.g., Elhalabi and Rice, Current Medicinal Chemistry, vol. 6, No. 2, 108-109, 1999) and/or use of “click chemistry” wherein the azido-containing fucose molecule is then complexed to an alkyne-containing molecule). (see, e.g., Sekhon, B S, Click chemistry: Current developments and applications in drug discovery, J. Pharm. Educ. Res. Vol. 3, Issue 1, 2012; e.g. Click-IT Fucose Alkyne, Thermo Fisher, At. No. C10264) In other embodiments, molecules covalently linked to the donor nucleotide fucose (i.e., GDP-fucose with covalent attachment of additional molecule(s)) can be stereospecifically added in a distinct pattern onto cell surface lactosaminyl glycans to endow CD44 with the ability to bind to desired ligand.

In some embodiments, the CD44 is modified ex vivo to provide structures receptive to ligands. In some embodiments, the CD44 is modified ex vivo via exofucosylation to enforce hematopoietic cell E-Selectin/L-Selectin Ligand (HCELL) expression. In some such embodiments, the modified CD44 may be ligated with one or more of a selectin (e.g., E-selectin and L-selectin) and monoclonal antibodies (mAbs) (e.g., the mAbs “CSLEX-1” and “HECA452”) effective to increase production of anti-inflammatory or immunomodulatory molecules in the CD44⁺ cells.

In some embodiments, the CD44 modifying enzymes, e.g., glycosidases, glycosyltransferases and fucosyltransferases are obtained from any convenient source, e.g., purified from eukaryotic or prokaryotic cells or obtained from commercial sources, including R&D Systems, SigmaAldrich, SCHsciences, and CarbExplore Research.

### Anti-Inflammatory or Immunomodulatory Molecules

In some embodiments, ligation of the CD44 is effective to produce elevated levels of at least one anti-inflammatory or immunomodulatory molecule in the CD44⁺ cells. As used herein, the term “anti-inflammatory molecule” and grammatical variation thereof means any molecule produced by a cell that acts to dampen inflammation, for example, by suppressing or restraining the action(s) of inflammatory effectors in a cell or tissue. As used herein, the term “immunomodulatory molecule” and grammatical variation thereof means any molecule produced by a cell that modulates an innate or adaptive immune response, excluding deleterious molecules that exacerbate an inflammatory disease or condition (e.g., pro-inflammatory molecules).

In some embodiments, the anti-inflammatory molecule is an anti-inflammatory cytokine. As used herein, the term “cytokine” includes, but is not limited to, leukocyte-generated peptides (e.g., lymphocyte-generated lymphokines, monocyte-produced monokines), chemokines, interferons, interleukins, adipocyte-secreted adipokines and muscle-generated myokines. In some embodiments, the anti-inflammatory molecules include, but are not limited to, Interleukin-10 (IL-10), TGF-β, IDO, nitric oxide (NO) metabolites, PGE₂ and combinations thereof. In some embodiments, ligation of the CD44 is effective to elevate production of at least one anti-inflammatory molecule at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to native population of cells, e.g., an untreated population of cells, such as an untreated population of CD44⁺ cells. In some embodiments, ligation of the CD44 is effective to elevate production of at least one immunomodulatory molecule at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of cells, e.g., an untreated population of cells, such as an untreated population of CD44⁺ cells.

In some embodiments, the anti-inflammatory and/or immunomodulatory molecule is released from the CD44⁺ cells via secratome. In some embodiments, the anti-inflammatory and/or immunomodulatory molecule is released from the CD44⁺ cells via extracellular vesicle (e.g., microvesicles and exosome). In some embodiments, the anti-inflammatory and/or immunomodulatory molecule is released from the CD44⁺ cells in soluble form. In some embodiments, the extracellular vesicles released by the cells in a media, such as microvesicles and exosomes, are isolated and/or purified from the media. In some embodiments, one or more extracellular vesicles may be isolated using a two-step protocol: (1) ExoQuick™ solution (Systems Biosciences) may be added to cell culture media at a volume of one to five, centrifuged at 1,500 g for 30 min, to form a pellet; and (2) ExoCap™ (JSR Life Sciences) composite reagent containing magnetic beads for CD9, CD63, and CD81 may be used to purify exosomes, which may then be eluted from beads using modified manufacturer's elution buffer (See, e.g., Cooper et al., Advances in Wound Care, vol. 7, no. 9, 299-308).

In some embodiments, the anti-inflammatory or immunomodulatory molecules produced by the CD44⁺ cells are effective to promote tissue repair in a subject. As used herein, the term “subject” and grammatical variation thereof, includes, but is not limited to, any mammal, such as a human, non-human primate, mouse, rat, dog, cat, horse, or cow. In some embodiments, the production of elevated level of anti-inflammatory cytokines (such as IL-10) by the CD44⁺ cells are effective to facilitate regenerative healing of damaged tissue in a subject. In some embodiments, the production of elevated level of anti-inflammatory cytokines (such as IL-10) by the CD44⁺ cells are effective to facilitate regenerative healing via one or more of promoting the production and deposition of extracellular matrix components in damaged tissue of a subject, modulating fibroblast function, modulating myofibroblast differentiation, and modulating endothelial progenitor cell survival and function. In some embodiments, ligation of the CD44 is effective to produce elevated levels of at least one anti-inflammatory or immunomodulatory molecules in the CD44⁺ cells that is effective to promote tissue repair in a subject. CD44⁺ Cells

According to some aspects, the present disclosure provides methods of making and using a modified population of CD44⁺ cells that have been modified ex vivo to increase production of one or more anti-inflammatory cytokines by interacting, treating or otherwise bringing CD44 into contact with a natural or artificial ligand (i.e., ligating the CD44). In some embodiments, the CD44⁺ cells comprise CD34⁻/CD44⁺/PSGL⁻ cells. In some embodiments, the CD44⁺ cells are human cells. In some embodiments, the CD44⁺ cells are non-human animal cells.

In some embodiments, the CD44⁺ cells are mesenchymal stem cells (MSCs), hematopoietic stem cells, tissue stem/progenitor cells (for example, a neural stem cell, myocyte stem cell or pulmonary stem cell), umbilical cord stem cells, or embryonic stem cells, induced pluripotent stem cells, differentiated progenitors derived from embryonic stem cells or from induced pluripotent stem cells, differentiated progenitors derived from adult stem cells, primary cells isolated from any tissue (e.g., blood, bone marrow, brain, liver, lung, gut, stomach, fat, muscle, testes, uterus, ovary, skin, spleen, eye, endocrine organ and bone), a culture-expanded progenitor cell population, a culture-expanded stem cell population, or a culture-expanded primary cell population.

In some embodiments, the CD44⁺ cells are mesenchymal stem cells. The term “mesenchymal stem cell” (MSC) refers to cells isolated from stroma, the connective tissue that is embedded within tissues and organs. Isolation of MSCs from diverse tissues has led to multiple names for these cells (e.g., if isolated from umbilical cord, they can have names such as “umbilical lining stem cells” or “Wharton jelly cells”), but, herein, all plastic-adherent cells derived from any tissue that possesses multipotency as defined by ability to differentiate into at least two of the following four cell lineages—osteoblast, adipocyte, chondrocyte, and/or fibroblast—will be considered to be an “MSC”. MSCs are also known as “multipotent stromal cells” or “mesenchymal stromal cells” or “mesenchymal precursor cells” or “mesenchymal lineage cells”, in each case, herein, such cells are considered “mesenchymal stem cells” and will be abbreviated as “MSCs.” MSCs characteristically express a panel of markers including, but not limited to, CD13, CD44, CD73, CD105. MSCs do not typically express either the CD34 or PSGL (CD162) markers. MSCs are postnatal stem cells capable of self-renewing and can differentiate into a variety of cells such as osteoblasts, chondrocytes, adipocytes, fibroblasts, and neural cells. MSCs typically express STRO-1, CD29, CD73, CD90, CD105, CD146, and SSEA4, but do not typically express hematopoietic cell markers, especially CD14 and CD34. However, MSCs derived from tissues other than marrow (e.g., from adipose tissue) and a subset of MSCs known as “pericytes” or “adventitial” cells can natively express CD34 (such cells may comprise the population known as “stromal vascular fraction”), and this marker is lost on culture-expansion. In some embodiments, the MSCs are cultured at low densities (i.e., less than 70% maximum confluency). An MSC according to the present disclosure may be unmodified or may be modified (e.g., by nucleic acid transfection to express a desired protein product of interest, by viral transduction, etc.). In some embodiments, the CD44⁺ cells are culture-expanded mesenchymal stem cells. In some embodiments, the CD44⁺ cells are culture-expanded mammalian adipose-derived mesenchymal stem cells (AdMSCs). In some embodiments, the CD44⁺ cells are culture-expanded human adipose-derived mesenchymal stem cells (hAdMSCs). In the present disclosure, the CD44⁺ cells may be culture expand using any appropriate method, including, e.g., the method disclosed in the Examples below.

In some embodiments, the CD44⁺ cell is a somatic human cell such as an epithelial cell (e.g., a skin cell), a hepatocyte (e.g. a primary hepatocyte), a neuronal cell (e.g. a primary neuronal cell), a myoblast (e.g. a primary myoblast), or a leukocyte. The CD44⁺ cell could be a human tissue progenitor cell or a stem cell (e.g., a mesenchymal stem cell). In some embodiments, the CD44⁺ cell type includes, but is not limited to, embryonic stem cells, adult stem cells, induced pluripotent stem cells, blood progenitor cells, tissue progenitor cells, stromal vascular fraction cells, or primary cells from any tissue or blood, e.g., epithelial, endothelial, neuronal, adipose, cardiac, skeletal muscle, fibroblast, immune cells (for example, dendritic cells, monocytes, macrophages, granulocytes, lymphocyte-type leukocytes (e.g., a lymphocyte such as a B-lymphocyte, a T-lymphocyte, or a subset of T-lymphocytes, such as regulatory lymphocyte (e.g., CD4⁺/CD25⁺/FOXP3⁺ cells, Breg cells, etc.), a naive T cell, a central memory T cell, an effector memory T cell, an effector T cell, NK cells, etc.), hepatic, splenic, lung, circulating blood cells, platelets, reproductive cells, gastrointestinal cells, renal cells, bone marrow cells, cardiac cells, endothelial cells, endocrine cells, skin cells, muscle cells, neuronal cells, and pancreatic cells. In some embodiments, the CD44⁺ cell can be an umbilical cord-derived stem cell, an embryonic stem cell, or a cell isolated from any tissue (such as a primary cell) including, but not limited to blood, bone marrow, brain, liver, lung, gut, stomach, fat, muscle, testes, uterus, ovary, skin, spleen, eye, endocrine organ and bone, and the like. In certain embodiments, the CD44⁺ cell can be culture-expanded and/or modified in vitro by introduction of any nucleic acid sequence encoding a protein of interest. The CD44⁺ cell can be derived from a tissue progenitor cell or a stem cell or a somatic cell (e.g., a monocyte-derived dendritic cell).

Where the CD44⁺ cell is maintained under in vitro conditions, conventional tissue culture conditions and methods can be used, and are known to those of skill in the art. Isolation and culture methods, and cell expansion methods, for various cells are well within the knowledge of one skilled in the art. Moreover, various CD44⁺ cells that contain nucleic acid encoding therapeutically advantageous protein products are also within the scope of the present disclosure (e.g., CAR-T cells (Casucci, 2013), nucleic acid modified cells, gene-modified cells, RNA-modified cells, etc. (See, e.g., Levy, 2013; Warren, 2010))

In addition, both heterogeneous and homogeneous CD44⁺ cell populations are contemplated for use with the methods and compositions of the present disclosure. In addition, CD44⁺ cells that are aggregates of cells, cells attached to or encapsulated within particles, cells within injectable delivery vehicles such as hydrogels, and cells attached to transplantable substrates (including scaffolds) or applied into tissue(s) that harbors scaffolds or transplantable substrates are contemplated for use with the methods and compositions of the present disclosure. Moreover, CD44⁺ cells according to the present disclosure may be used in combination with tissue proliferative and/or enhancing agents and/or anti-inflammatory agents (e.g., growth factors, cytokines, prostaglandins, trophic agents, Resolvins, NSAIDS, steroids, etc.) Inflammatory Disorder Treatments

According to some embodiments, the present disclosure provides, inter alia, a method of treating a disease or disorder associated with elevated levels of at least one pro-inflammatory cytokine in a subject comprising administering to the subject a composition as disclosed herein.

In some embodiments, the disease or disorder associated with elevated levels of at least one pro-inflammatory cytokine either locally or systemically is selected from neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), ocular diseases (e.g., Behcet's syndrome, macular degeneration, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, SIRS, acute respiratory disease syndrome secondary to bacteria, fungi or viruses (e.g., influenza, coronavirus, COVID-19, SARS, MERS, etc.)), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.). In some embodiments, the disease or disorder associated with elevated levels of at least one pro-inflammatory cytokine is a cytokine storm. In some embodiments, the disease or disorder associated with elevated levels of at least one pro-inflammatory cytokine is graft versus host (GvH) disease. In some embodiments, the disease or disorder associated with elevated levels of at least one pro-inflammatory cytokine is multi-system inflammatory syndrome. In some embodiments, the disease or disorder associated with elevated levels of at least one pro-inflammatory cytokine is COVID-19. In some embodiments, the E-selectin or L-selectin-primed cells are further exofucosylated prior to administration to the subject. In some embodiments, the HA-primed cells are exofucosylated prior to administration to the subject.

The compositions, including cell populations, pharmaceutical compositions and methods disclosed herein are useful for the treatment of a disease associated with one or more of neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, SIRS, acute respiratory disease syndrome secondary to bacteria, fungi or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.). In some embodiments, the compositions, including cell populations, pharmaceutical compositions and methods disclosed herein are useful for the treatment of a disease associated with a cytokine storm. As used herein, the term “cytokine storm” and grammatical variation thereof, means any systemic inflammatory response or severe immune reaction in which cytokines are released in a subjects body and that may cause one or more of fever, inflammation, fatigue, nausea, organ failure, and death. In some embodiments, the compositions, including cell populations, pharmaceutical compositions and methods disclosed herein are useful for effecting immunohomeostasis in a subject. In some embodiments, the compositions, including cell populations, pharmaceutical compositions and methods disclosed herein are useful for the treatment of graft versus host (GvH) disease. In some embodiments, the compositions, including cell populations, pharmaceutical compositions and methods disclosed herein are useful for the treatment of respiratory diseases, especially diseases associated with single-strand enveloped RNA viruses, such as those belonging to the family Coronaviridae, including coronaviruses, such as SARS, MERS, and SARS-CoV2 also known as COVID-19. In some embodiments, the compositions, including cell populations, pharmaceutical compositions and methods disclosed herein are useful for the treatment of a disease or disorder in a human subject.

According to some embodiments, the compositions and methods disclosed herein are useful for decreasing plasma levels of at least one pro-inflammatory molecule in a subject. As used herein, the term “pro-inflammatory molecule” and grammatical variation thereof means any molecule produced by a cell that acts to amplify inflammation. In some embodiments, the pro-inflammatory molecule is a pro-inflammatory cytokine. In some embodiments, the compositions and methods disclosed herein are useful for decreasing plasma levels of at least one pro-inflammatory molecule in a subject when administered to the subject. In some embodiments, the at least one pro-inflammatory molecule comprises a group selected from IFNγ, TNFα, IL-1α, IL-1β, IL-6, IL-12, IL-17 and combinations thereof. In some embodiments, the compositions and methods disclosed herein are useful for decreasing plasma levels of at least one pro-inflammatory molecule in a subject when administered to the subject by an amount of at least 2%, 5%, 10%, 25%, 50%, 80%, or 90%.

According to some embodiments, the compositions and methods disclosed herein are useful for increasing one or more anti-inflammatory molecules systemically or locally in a subject. According to some embodiments, the compositions and methods disclosed herein are useful for increasing the level of anti-inflammatory molecules in the plasma of a subject and/or within damaged/inflamed tissue (i.e., a lesional site) of a subject. In some embodiments, the level of anti-inflammatory molecule (such as IL-10) within the affected tissue and/or within the plasma is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to its baseline level. In some embodiments, the increase in at least one anti-inflammatory molecule in lesional sites or plasma, and the decrease in levels of at least one pro-inflammatory molecule in lesional sites or plasma is observed for a prolonged period of time. In some embodiments, the prolonged period of time is for at least 10 days, at least 20 days or at least 30 days.

### Low Cell Dose

Given the surprisingly increased potency of the compositions, including cell populations and pharmaceutical compositions disclosed herein, it is possible to use approximately ⅕ (one fifth) the number of modified cells in the present disclosure compared to conventional cell-based therapies to achieve the same or better level of therapeutic benefit. Thus, according to some embodiments, the compositions and methods disclosed herein are effective to produce or induce the production of enhanced levels of anti-inflammatory and/or immunomodulatory molecules such that low doses of cells, relative to a conventional therapy, may be administered to effectively treat a disease associated with an inflammatory disease or disorder. In some embodiments, the low dose compositions, including cell populations, pharmaceutical compositions and use of such compositions in the methods disclosed herein comprises at least about 50,000 primed cells/kg (based on the weight of the subject), at least about 200,000 primed cells/kg (based on the weight of the subject), at least about 400,000 primed cells/kg (based on the weight of the subject), at least about 500,000 primed cells/kg (based on the weight of the subject), at least about 600,000 primed cells/kg (based on the weight of the subject), at least about 700,000 primed cells/kg (based on the weight of the subject), at least about 800,000 primed cells/kg (based on the weight of the subject), or at least about 900,000 primed cells/kg (based on the weight of the subject). In some embodiments, the low dose compositions, including cell populations, pharmaceutical compositions and use of such compositions in the methods disclosed herein comprises at least about 50,000 primed cells, at least about 200,000 primed cells, at least about 400,000 primed cells, at least about 500,000 primed cells, at least about 600,000 primed cells, at least about 700,000 primed cells, at least about 800,000 primed cells, or at least about 900,000 primed cells. In some embodiments, the low dose compositions, including cell populations, pharmaceutical compositions and use of such compositions in the methods disclosed herein comprise less than 200,000 primed cells.

### Exofucosylation

According to some embodiments, cells of the present disclosure are contacted with a glycosyltransferase to enforce a glycan on the cell surface. In some embodiments, the glycosylstranferase is a human glycosyltransferase. In some embodiments, the glycosylstranferase is a non-human glycosyltransferase. According to some embodiments, fucosylated lactosaminyl glycans are enforced by a member of the α(1,3)-fucosyltransferase family. The human α(1,3)-fucosyltransferase family includes Fucosyltransferase III (also called FTIII, FT3, FUTIII, or FUT3), Fucosyltransferase IV (also called FTIV, FT4, FUTIV, or FUT4), Fucosyltransferase V (also called FTV, FT5, FUTV, or FUT5), Fucosyltransferase VI (also called FTVI, FT6, FUTVI, or FUT6), Fucosyltransferase VII (also called FTVII, FT7, FUTVII, or FUT7), Fucosyltransferase IX (also called FTIX, FT9, FUTIX, or FUT9), and variants thereof. The cDNA/protein sequences for the α(1,3)-fucosyltransferase family are as follows:

| Name | GenBank Acc. No. | ID NO: |
|---|---|---|
| Fucosyltransferase III | BC108675 | SEQ ID NO: 1 (AA) |
| (FUT3; FT3) | | SEQ ID NO: 2 (cDNA) |
| Fucosyltransferase IV | BC136374 | Long |
| (FUT4; FT4) | | SEQ ID NO: 3 (AA) |
| | | SEQ ID NO: 4 (cDNA) |
| | | Short |
| | | SEQ ID NO: 5 (AA) |
| | | SEQ ID NO: 6 (cDNA) |
| Fucosyltransferase V | BC140905 | SEQ ID NO: 7 (AA) |
| (FUT5; FT5) | | SEQ ID NO: 8 (cDNA) |
| Fucosyltransferase VI | BC061700 | SEQ ID NO: 9 (AA) |
| (FUT6; FT6) | | SEQ ID NO: 10 (cDNA) |
| Fucosyltransferase VII | BC074746 | SEQ ID NO: 11 (AA) |
| (FUT7; FT7) | | SEQ ID NO: 12 (cDNA) |

As used herein, the notation for a fucosyltransferase should not be construed as limiting to the nucleotide sequence or the amino acid sequence. For example, the notation of Fucosyltransferase VII, FTVII, FT7, FUTVII or FUT7 are used interchangeably as meaning the nucleotide, amino acid sequence, or both, of Fucosyltransferase VII. According to some embodiments, cells are contacted by one or more of the α(1,3)-fucosyltransferase family members to enforce fucosylated lactosaminyl glycans.

In some embodiments, fragments of α(1,3)-fucosyltransferase family members are contacted with a cell. For example, a peptide/nucleotide having at least 50%, 60%, 70%, 80%, 90%, 95%, 96%, 97%, 98%, or 99% identity to an α(1,3)-fucosyltransferase family member is contacted with a cell. As used herein, the term “identity” and grammatical versions thereof means the extent to which two nucleotide or amino acid sequences have the same residues at the same positions in an alignment. Percent (%) identity is calculated by multiplying the number of matches in a sequence alignment by 100 and dividing by the length of the aligned region, including internal gaps.

In some embodiments, the cells may be contacted with the desired fucosyltransferase via exofucosyltation using, for example, the methods disclosed herein. U.S. Pat. Nos. 7,875,585 and 8,084,236, (which disclosures are expressly incorporated by reference as if recited in full herein) provide non-limiting examples of compositions and methods for ex vivo modification of cell surface glycans on a viable cell, which may be used to enforce expression of fucosylated lactosaminyl glycans (e.g. HCELL) on a cell according to the present disclosure. In some embodiments, the cells may be contacted with a purified glycosyltransferase polypeptide and a physiologically acceptable solution, for use together with appropriate donor nucleotide sugars in reaction buffers and reaction conditions specifically formulated to retain cell viability. In some embodiments, the physiologically acceptable solution may be free or substantially free of divalent metal co-factors, to such extent that cell viability is not compromised. In these and other embodiments, the cells may be contacted with a solution that is also free or substantially free of stabilizer compounds such as for example, glycerol, again, to such extent that cell viability is not compromised. Glycosyltransferases of the present disclosure include for example, one or more fucosyltransferase. In certain embodiments, the fucosyltransferase is an α(1,3)-fucosyltransferase such as an α(1,3)-fucosyltransferase III, α(1,3)-fucosyltransferase IV, an α(1,3)-fucosyltransferase V, an α(1,3)-fucosyltransferase VI, or an α(1,3)-fucosyltransferase VII.

According to some embodiments the human or mammalian cells of the present disclosure may be contacted with a desired fucosyltransferase by transfecting a DNA or RNA nucleotide sequence encoding the desired fucosyltransferase into the cell. According to some embodiments, modified RNA (modRNA) encoding the relevant α(1,3)-FT transcripts is used to enforce the desired pattern of fucosylated lactosaminyl glycans. (Levy, 2013; Warren 2010) In some embodiments, the transfected nucleotide sequence encodes a full length or partial peptide sequence of the desired fucosyltransferase. In some embodiments, the nucleotide sequence encodes a naturally existing isoform of a fucosyltransferase. (See, e.g., Mondal N. et al. Distinct human α(1,3)-fucosyltransferases drive Lewis-X/sialyl Lewis-X assembly in human cells. J Biol Chem. 2018; 293(19):7300-7314.)

According to some embodiments, the cells may be contacted with the desired fucosyltansferase by transfecting the cells with a recombinant DNA or RNA molecule. (Levy 2013, Warren 2010) As used herein, the term “recombinant DNA or RNA” means a DNA or RNA molecule formed through recombination methods to splice fragments of DNA or RNA from a different source or from different parts of the same source. In some embodiments the recombinant DNA may comprise a plasmid vector, which controls expression of the DNA in the cell. Proteins, such as the enzymes disclosed herein, which are encoded by recombinant DNA or RNA are recombinant proteins.

In some embodiments, glycans are modified on the surface of a cell by contacting a population of cells with one or more glycosyltransferase compositions described above. In some embodiments, the cells are contacted with the glycosyltransferase composition together with an appropriate nucleotide sugar donor (e.g., GDP-fucose, CMP-sialic acid) under conditions in which the glycosyltransferase has enzymatic activity. For example, cells may be incubated for 60 min at 37° C. in fucosyltransferase reaction buffer composed of Hank's Balanced Salt Solution (HBSS) (without Ca²⁺ and Mg²⁺) (Lonza) containing 20 mM HEPES (Lonza), 0.1% human serum albumin (HSA) (Grifols, Barcelona, Spain), 30 μg/ml fucosyltranferase, and 1 mM GDP-fucose. Glycan modification according to this method results in cells according to the present disclosure that have at least 70%, 75%, 80%, 85%, 90%, 95%, 96%, 97%, 98%, 99% or more viability at 24 hours or more after treatment. In one embodiment, for example, the cells of the present disclosure have at least 70% viability at 48 hours after treatment. In one such embodiment, for example, the cells of the present disclosure have at least 75% viability at 48 hours after treatment. In one embodiment, for example, the cells of the present disclosure have at least 80% viability at 48 hours after treatment. In addition, the phenotype of the cells of the present disclosure (other than the glycan modification) is preferably preserved after treatment. By preserved phenotype, it is meant the cell of the present disclosure maintains its native function and/or activity. For example, if the cell of the present disclosure is a stem cell it retains its potency, i.e., its relevant totipotency or pluripotency or multipotency or unipotency, as would be characteristic of that particular stem cell type.

According to some embodiments, glycosyltransferases are contacted with cells of the present disclosure in the absence of (or substantially in the absence of) divalent metal co-factors (e.g. divalent cations such as manganese, magnesium, calcium, zinc, cobalt or nickel) and stabilizers such as glycerol. In some embodiments, a purified glycosyltransferase polypeptide and a physiologically acceptable solution free or substantially free of divalent metal co-factors is used to enforce a desired glycosylation pattern. Such a composition is free or substantially free of stabilizer compounds such as for example, glycerol, or the composition contains stabilizers at levels that do not affect cell viability. The glycosyltransferases used with solutions that are free or substantially free of divalent metal cofactors include for example, α(1,3)-fucosyltransferases such as an α 1,3 fucosyltransferase III, α 1,3 fucosyltransferase IV, an α 1,3 fucosyltransferase VI, or an α 1,3 fucosyltransferase VII. According to some embodiments, the glycosyltransferase is biologically active. As used herein “biologically active” means that the glycosyltransferase is capable of transferring a sugar molecule from a donor to acceptor. For example, a glycosyltransferase according to the present disclosure is capable of transferring 0.1, 0.2, 0.3, 0.4, 0.5, 1.0, 1.5, 2.0, 2.5, 5, 10 or more pmoles of sugar per minute at pH 6.5 at 37° C. In some embodiments, the contacting of a glycosyltranferase with a cell occurs in a physiologically acceptable solution, which is any solution that does not cause cell damage, e.g. death. For example, the viability of the cell is at least 80%, 85%, 90%, 95%, 96%, 97%, 98%, 99% or more after treatment with the compositions of the invention. Suitable physiologically acceptable solutions include, for example, Hank's Balanced Salt Solution (HBSS), Dulbecco's Modified Eagle Medium (DMEM), a Good's buffer such as a HEPES buffer, a 2-Morpholinoethanesulfonic acid (MES) buffer, or phosphate buffered saline (PBS).

### Administration of Cells

Administration of compositions, pharmaceutical compositions, including cell populations disclosed herein for therapeutic indications can be achieved in a variety of ways, in each case as clinically warranted, using a variety of anatomic access devices, a variety of administration devices, and a variety of anatomic approaches, with or without support of anatomic imaging modalities (e.g., radiologic, MRI, ultrasound, etc.) or mapping technologies (e.g., epiphysiologic mapping procedures, electromyographic procedures, electrodiagnostic procedures, etc.). The compositions, pharmaceutical compositions and cell populations of the present disclosure can be administered systemically, via either peripheral vascular access (e.g., intravenous placement, peripheral venous access devices, etc.) or central vascular access (e.g., central venous catheter/devices, arterial access devices/approaches, etc.). The compositions, pharmaceutical compositions and cell populations of the present disclosure can be delivered intravascularly into anatomic feeder vessels of an intended tissue site using catheter-based approaches or other vascular access devices (e.g., cardiac catheterization, etc.) that will deliver a vascular bolus of cells to the intended site. The compositions, pharmaceutical compositions and cell populations of the present disclosure can be introduced into the spinal canal and/or intraventricularly intrathecally, into the subarachnoid space to distribute within cerebrospinal fluid and/or within the ventricles). The compositions, pharmaceutical compositions and cell populations of the present disclosure can be administered directly into body cavities or anatomic compartments by either catheter-based approaches or direct injection (e.g., intraperitoneal, intrapleural, intrapericardial, intravesicularly (e.g., into bladder, into gall bladder, into bone marrow, into biliary system (including biliary duct and pancreatic duct network), intraurethrally, via renal pelvis/intraureteral approaches, intravaginally, etc.)). The compositions, pharmaceutical compositions and cell populations of the present disclosure can be introduced by direct local tissue injection, using either intravascular approaches (e.g., endomyocardial injection), or percutaneous approaches, or via surgical exposure/approaches to the tissue, or via laparoscopic/thoracoscopic/endoscopic/colonoscopic approaches, or directly into anatomically accessible tissue sites and/or guided by imaging techniques (e.g., intra-articular, intra-ocular, into spinal discs and other cartilage, into bones, into muscles, into skin, into connective tissues, and into relevant tissues/organs such as central nervous system, peripheral nervous system, heart, liver, kidneys, spleen, joints, eye, etc.). The compositions, pharmaceutical compositions and cell populations of the present disclosure can also be placed directly onto relevant tissue surfaces/sites (e.g., placement onto tissue directly, onto ulcers, onto burn surfaces, onto serosal or mucosal surfaces, onto epicardium, etc.). The compositions, pharmaceutical compositions and cell populations of the present disclosure can also administered into tissue or structural support devices (e.g., tissue scaffold devices and/or embedded within scaffolds placed into tissues, etc.), and/or administered in gels, and/or administered together with enhancing agents (e.g., admixed with supportive cells, cytokines, growth factors, resolvins, anti-inflammatory agents, etc.).

According to some embodiments, the compositions, pharmaceutical compositions and cell populations of the present disclosure are administered to the subject with an enforced expression of glycosylation. According to some embodiments, the enforced glycosylation on the surface of administered cells will aid in revascularization, in host defense (e.g., against infection or cancer) and/or in tissue repair/regeneration and/or mediate immunomodulatory processes that will dampen inflammation and/or prevent inflammation. According to some embodiments, the enforced glycosylation pattern guides delivery of intravascularly administered cells to sites of inflammation by mediating binding of blood-borne cells to vascular E-selectin expressed on endothelial cells at sites of inflammation. Moreover, whether cells are administered systemically, intravascularly, into the spinal canal and/or intraventricularly intrathecally, into the subarachnoid space to distribute within cerebrospinal fluid), directly into body cavities or compartments, by direct local tissue injection, or by placement onto relevant tissue surfaces/sites, the enforced expression of ligands for E-selectin and/or L-selectin on administered cells promotes lodgment of cells within the affected tissue milieu, in apposition to cells bearing E-selectin (i.e., endothelial cells) and/or L-selectin (i.e., leukocytes), respectively, within the target site. Thus, the spatial distribution and localization of administered cells within the target tissue is modulated by the enforced glycosylation on administered cells.

Particularly, the colonization of a desired cell type at a site of inflammation occurs as a result of the enforced glycosylation on the administered cells, such that the administered cells have augmented binding to E-selectin, thereby promoting the systemic delivery of the desired cells and/or the lodgement of cells when injected directly into the affected site. For example, the enforced glycosylation of E-selectin ligands (e.g., HCELL) is advantageously capable of anchoring directly injected cells within E-selectin-expressing vessels at sites of inflammation, tissue injury, or cancer. Thus, the present methods augment efficiency in the delivery of relevant cells at or to a site of inflammation, tissue injury, or cancer, including, for example, the capacity to deliver immunomodulatory cells (e.g., mesenchymal stem cells).

In some embodiments the disease, disorder, or medical condition having associated inflammation can be treated using the instant methods even in the absence of differentiation of the cell population in the subject. That is, there are trophic effects of administered cells at the site of inflammation without persistent engraftment and/or repopulation of the administered cells, irrespective of the type of tissue involved. These trophic effects include release of cytokines/growth factors that promote revascularization (e.g., VEGF), that promote tissue repair (e.g., TGF-β), that are immunomodulatory (e.g., IL-10), that stimulate growth/proliferation of tissue-resident progenitors (e.g., SCF, LIF, etc) and many other tissue-reparative processes (e.g., mitochondria delivery to cells). In addition, administered cells (e.g., MSCs) may have potent immunomodulatory properties, including direct suppression of activated lymphocytes (e.g., via expression of PDL-1).

According to some aspects, the present disclosure provides a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells. According to some aspects, the present disclosure provides a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory cytokine; or (2) modified ex vivo via treatment with hyaluronic acid (HA) for a period of time sufficient to prime the cells to produce interleukin-10 (IL-10) and at least one additional anti-inflammatory cytokine, wherein the modified cells of (1) or (2) produce elevated levels of IL-10 and at least one additional anti-inflammatory cytokine relative to a native population of CD44⁺ cells.

In some embodiments, the conditioned media may be administered to a subject by any of the methods and routes of administration disclosed herein. In some embodiments, the conditioned media may be administered to a subject in aerosolized form.

In some embodiments, the compositions, cell populations and pharmaceutical compositions and methods disclosed herein are useful for the treatment of respiratory diseases, such as diseases associated with single-strand enveloped RNA viruses, such as those belonging to the family Coronaviridae, including coronaviruses, such as SARS, MERS, and SARS-CoV2 also known as COVID-19. In some embodiments, the compositions, cell populations and pharmaceutical compositions disclosed herein are effective to treat COVID-19 in human subjects. In some embodiments, the compositions, cell populations and pharmaceutical compositions disclosed herein are effective to treat acute respiratory distress syndrome (ARDS) and/or who are experiencing a cytokine storm. In some embodiments, the compositions, cell populations and pharmaceutical compositions disclosed herein are administered to treat a subject by any of the methods and routes of administration disclosed herein, including in aerosolized form directly into the lungs of a subject. In some embodiments, cell populations or cell secretome products may be aerosolized for delivery to a subject, such as for delivery to a subject's lungs. A variety of conditions can be utilized to aerosolize cells or cell secretome products. In some embodiments, cells are suspended in saline (e.g., 1-5 mL) and aerosolized at a pressure of 3-100 psi for 1-15 minutes, or until cells begin to rupture and/or die. Any form of aerosolizer can be used to deliver cells to a subject's lungs provided the cells can be delivered substantially without damage. In some embodiments, the compositions, cell populations, secretome products and pharmaceutical compositions disclosed herein can be aerosolized in particles of various sizes (e.g., nanoparticles). For example, in some embodiments, an aerosolizer can be used that aerosolizes to a particle size of about 2 microns to about 50 microns. In some embodiments, an aerosolizer can be used that aerosolizes to a particle size of about 4 microns to about 30 microns. In some embodiments, an aerosolizer can be used that aerosolizes to a particle size of about 6 microns to about 20 microns. In some embodiments, an aerosolizer can be used that aerosolizes to a particle size of about 6 microns to about 200 microns.

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the methods of the present invention. These examples are illustrative only and are not intended to limit the scope of the invention in any way.

### Example 1—Materials and Methods

### Mice

BALB/c (H-2^{d}) donors and C57BL/6J (H-2^{b}) recipient mice were purchased from Envigo (Huntingdon, United Kingdom), whereas p-actin-GFP transgenic C57BL/6-Tg(CAG-EGFP) was from The Jackson Laboratory (Bar Harbor, ME, United States). All animal procedures were approved by the Institutional Animal Care and Use Committee at University of Murcia (Murcia, Spain) and performed according to Institutional guidelines (approved protocol A13150201).

### Mesenchymal Stem Cell Isolation and Culture

mAdMSCs, hAdMSCs and hBMMSCs were isolated as described previously (Valencia et al., 2016; Yanez et al., 2006). In brief, mAdMSCs from C57BL/6 or C57BL/6-Tg (CAG-EGFP) mice, or hAdMSCs and hBMMSCs from healthy human donors, were flask-seeded in DMEM low glucose medium (Gibco, Carlsbad, CA, United States) supplemented with 15% fetal bovine serum (Gibco), 1% L-glutamine (Lonza, Basel, Switzerland), 100 U/ml penicillin, and 100 μg/ml streptomycin (Lonza) (complete medium). MSCs in culture passages 3-4 were used for experiments. The Institutional Review Board of the University Hospital Virgen de la Arrixaca (Murcia, Spain) approved the protocols used to obtain and process all human samples. As needed, written informed consent was obtained from donors as per Helsinki Declaration guidelines.

### Mesenchymal Stem Cell Characterization and Multipotent Differentiation Assays

The immunophenotype of cultured mAdMSCs was analyzed by flow cytometry using anti-mouse antibodies directed to markers CD44, CD73, CD90, CD105, CD29, Sca-1, CD166, CD106, CD34, CD45, CXCR4, c-Kit, CD80, CD86 (all from BioLegend, San Diego, CA, United States) and HECA452 (BD Biosciences, San Jose, CA, United States), together with their specific control isotypes. Recombinant mouse E-selectin-human Fc chimera (mE-Ig) was purchased from R&D Systems (Minneapolis, MN, United States). The immunophenotypic characterization of cultured hMSCs was performed using an MSC phenotyping kit (Miltenyi Biotec, Bergisch Gladbach, Germany) that includes antibodies specific for human CD73, CD90, CD105, CD14, CD20, CD34 and CD45. Briefly, mMSCs and hMSCs were detached with TripLE Select solution (Gibco), washed, and resuspended in phosphate-buffered saline (Gibco) containing 1% bovine serum albumin (Sigma-Aldrich, St. Louis, MO, United States). Thereafter, cells were incubated with the indicated antibodies for 30 min in the dark at 4° C., washed and analyzed in a FACSCanto flow cytometer (BD Biosciences).

For multipotent differentiation assays, mAdMSCs were differentiated toward the adipogenic, osteogenic and chondrogenic mesodermal lineages as reported previously (Tormin et al., 2009). Adipogenic differentiation was induced after culturing cells in complete medium and adipogenic supplement containing hydrocortisone, isobutylmethylxantine and indomethacin, following the manufacturer's instructions (mouse mesenchymal stem cell functional identification kit (R&D Systems). After 14 days, cells were fixed with 4% paraformaldehide in PBS and stained with Oil Red O solution (Sigma-Aldrich) to detect the accumulation of neutral lipids in fat vacuoles. In addition, expression of fatty acid binding protein 4 (FABP4) was detected by immunofluorescence using a polyclonal goat anti-mouse FABP4 antibody (R&D Systems). For osteogenic differentiation, mAdMSCs were cultured in complete medium and osteogenic supplement containing ascorbate-phosphate, β-glycerophosphate and recombinant human bone morphogenetic protein-2 (BMP-2). After 21 days, cells were fixed with 4% paraformaldehide in PBS and stained with Alizarin Red (Sigma-Aldrich) to detection of calcium depositions in the cultures. Also, alkaline phosphate activity was assessed by staining with SigmaFast™ BCIP-NBT (Sigma-Aldrich). Chondrogenic differentiation was accomplished by culturing mAdMSCs for 21 days in DMEM medium supplemented with dexamethasone, ascorbate-phosphate, proline, pyruvate, recombinant human TGFβ3, and 1% ITS supplement (R&D Systems). Afterwards, the cells were fixed with 4% paraformaldehide in PBS and stained with Alcian blue (Sigma-Aldrich) to detect cartilage mucopolysaccharides and glycosaminoglycans. Also, expression of collagen II was detected by immunofluorescence using a polyclonal sheep anti-mouse collagen II antibody (R&D Systems).

### Ex Vivo Fucosyltransferase VII Treatment

mAdMSCs were derived from C57BL/6 mice, the recipient strain for MHC-mismatched HSCT, whereas hAdMSCs and hBMMSCs were isolated from healthy human donors. Fucose was stereoselectively installed onto sialyllactosaminyl glycans of CD44 using an α(1,3)-linkage-specific fucosyltransferase, fucosyltransferase VII (FTVII; obtained from R&D Systems), in presence of donor fucose substrate (GDP-fucose; Sigma Aldrich): MSCs were resuspended at 2×10⁷ cells/ml and incubated for 60 min at 37° C. in FTVII reaction buffer composed of Hank's Balanced Salt Solution (HBSS) (without Ca²⁺ and Mg²⁺) (Lonza) containing 20 mM HEPES (Lonza), 0.1% human serum albumin (HSA) (Grifols, Barcelona, Spain), 30 μg/ml FTVII (R&D Systems), and 1 mM GDP-fucose (Fucosylation-modified, “FucmAdMSCs”). Controls consisted of MSCs treated with reaction buffer alone (i.e., Unmodified MSCs, “UmAdMSCs” or, “UhAdMSCs” and “UhBMMSCs”).

Exofucosylation efficacy was measured by analysis of HECA452 antibody staining and murine E-selectin-human Fc chimera (mE-Ig; from R&D Systems) binding by flow cytometry and western blot. In some experiments, FucmAdMSCs were digested with bromelain (Sigma-Aldrich) and proteinase K (Roche Diagnostics, Basel, Switzerland), as previously reported (Abdi et al., 2015).

### Western Blot

FTVII-treated and untreated control MSCs were homogenized with lysis buffer (Pro-Prep protein extraction solution, iNtRON Biotechnologies, Sevilla, Spain), containing protease inhibitor cocktail (Thermo Scientific, Waltham, MA, United States). All whole cell lysates were resolved by SDS-PAGE in 9% polyacrylamide gels. The amount of lysate in each lane was normalized to cell number for each experiment. After transferring to PVDF blotting membranes (GE Healthcare Life Science, Barcelona, Spain), samples were immunoblotted by incubation with mE-Ig chimera or CD44 primary antibody (BD Biosciences) and horseradish peroxidase-conjugated secondary antibodies, as previously described (Abdi et al., 2015). Blots were visualized using ECL Prime Western Blotting Detection Reagent (GE Healthcare Life Science).

### Mitogen Proliferative Assays

Mitogen proliferative assays were performed as described previously (Yanez et al., 2006; Yanez et al., 2010). Briefly, splenocytes were isolated from C57BL/6 and BALB/c mice spleen cell suspensions by passage through 40-μm nylon cell strainers (Becton Dickinson), followed by red blood lysis with 0.83% ammonium chloride in 0.01 M Tris-HCl buffer pH 7.5 (Sigma-Aldrich). To induce splenocyte proliferation, 1 ×10⁵ splenocytes were resuspended in RPMI 1640 medium (Gibco, Carlsbad, CA, United States) supplemented with 10% FBS (proliferation medium) and treated with 10 μg/ml concanavalin A (ConA) (Sigma-Aldrich). To assess for effect(s) on splenocyte proliferation, MSCs were resuspended in complete medium and seeded in wells together with splenocytes at decreasing ratios of MSC:splenocyte (from 1:1 to 1:100). After 3 days of MSC:splenocyte co-cultures, splenocyte proliferation was measured using an ELISA BrdU colorimetric kit (Roche Diagnostics). In brief, BrdU labeling reagent was added to wells 16 h before determination. Then, cells were fixed, DNA denatured and incubated with an anti-BrdU-POD antibody. After washing and substrate addition, absorbance was measured. In some experiments, UmAdMSCs and FucmAdMSCs, or FuchAdMSCs and FuchBMMSCs, were treated with sialidase from Vibrio cholerae (0.1 U/ml, Roche Diagnostics) to remove terminal sialic acids (i.e., sialUmAdMSCs and sialFucmAdMSCs, or sialFuchAdMSCs and sialFuchBMMSCs). As indicated, mMSCs or hMSCs were cultured for 24 h or 72 h with different concentrations of mE-Ig chimera or of hyaluronic acid (HA, from rooster comb; Sigma-Aldrich) for 24 h or 72 h following described protocols (Burdick et al., 2006; Thankamony and Sackstein, 2011). Briefly, mE-Ig or HA were immobilized on plates. HA-coated plates were incubated with 3% BSA in DMEM medium to block non-specific interactions. Thereafter, mAdMSCs or hMSCs were cultured in cell adhesion media consisting of HBSS containing 2 mM CaCl₂, 10 mM HEPES, 0.2% BSA and 1 mM sodium pyruvate (for mE-Ig), or DMEM medium containing 10 mM HEPES, 0.2% BSA and 1 mM sodium pyruvate (for HA), respectively. To block CD44/HCELL interactions to HA, a purified rat anti-mouse CD44 antibody (clone KM114, Santa Cruz Biotechnology, Dallas, TX, United States) were employed. In some experiments, cultures of mAdMSCs seeded on E-selectin and/or HA for 72 h were washed, then co-cultured in presence of continuous E-selectin and/or HA with murine splenocytes (at MSC:splenocyte ratio of 1:20) for 72 h in presence of ConA, and splenocyte proliferation was assessed.

To examine the contribution of different anti-inflammatory molecules in the immunosuppressive properties of mAdMSCs, the TGFβ inhibitor SB-431542 (final concentration (Cf)=10 μM), the IDO inhibitor 1-methyl-DL-tryptophan (Cf=1 μM) or the iNOS inhibitor N^{G}-monomethyl-L-arginine (Cf=1 mM) (all from Sigma-Aldrich) was added at the beginning of co-culture with splenocytes. In some experiments, hAdMSCs and hBMMSCs were adhered to E-selectin or HA for 72 h, washed, co-cultured at MSC:T cell ratio of 1:20 for 72 h with human peripheral blood T cells in presence of phytohemagglutinin (PHA, Sigma-Aldrich) and supernatants recollected for immunomodulatory molecules analysis.

aGvHD Model

Fully MHC-mismatched allo-HSCT was performed by transplanting bone marrow cells from donor BALB/c mice into 10-week-old C57BL/6 recipients previously irradiated with a potentially lethal dose of 10 Gy divided into two doses of 5 Gy spaced 24 hours apart (days −1 and +0). On day +0 recipient mice were transplanted intravenously with 1 ×10⁷ bone marrow cells from donor mice, either without (i.e., whereby aGvHD did not develop) or with 1.5 ×10⁷ donor splenocytes to induce aGvHD. Those mice treated with mAdMSC received intravenous infusions of 5×10⁴ recipient-type UmAdMSC or FucmAdMSCs on days 0, +7, and +14 post-transplantation. Survival of animals after transplantation was monitored daily whereas clinical aGvHD was assessed using a previously described scoring system (Hill et al., 1997).

### Histopathology Analysis

Histopathological changes of aGvHD were analyzed in at least two distant areas of liver, gut (colon), and skin as described previously (Gatza et al., 2008; Hill et al., 1997) by a single pathologist blinded to the treatment groups. Samples from all organs were collected and fixed in 4% neutral buffered formaldehyde for 24 h, processed and paraffin-embedded. Three-μm-thick sections were then obtained and stained with a standard hematoxylin and eosin (H&E) staining for routine histopathological analysis. The skin histopathologic lesions were graded as follows: grade 0 (normal), grade I (slight vacuolar degeneration of epidermal basal cells), grade II (scattered individual apoptotic epidermal basal cells and spongiosis), grade III (separation of dermo-epidermal junction) and grade IV (diffuse and severe ulceration, extensive destruction of epidermis). The scoring system for gut was: grade 0 (normal), grade I (scattered individual apoptotic cells and inflammatory cell infiltrate), grade II (crypt epithelial cell apoptosis, villous blunting, exploding crypts), grade III (focal mucosal ulceration and moderate villous atrophy) and grade IV (diffuse and severe mucosal ulceration). Histopathologic changes of liver sections were scored as: grade 0 (normal), grade I (epithelial damage and <25% bile ducts affected), grade II (epithelial damage and 25-49% bile ducts affected), grade III (epithelial damage and 50-74% bile ducts affected) and grade IV (epithelial damage and >75% bile ducts affected). In addition, polymorphonuclear neutrophils (PMNs) were identified on H&E stained sections on basis to its morphological features (segmented nuclei).

A standard indirect ABC immunohistochemical staining was performed in sections from all organs. Briefly, after deparaffination, rehydration, antigen demasking and peroxidase-blocking, sections were incubated with a polyclonal rabbit anti-CD3 antibody (Agilent Technologies, Santa Clara, CA, United States) for 1 h at 37° C. In other experiments and to detect the distribution of transplanted GFP-expressing mAdMSCs, sections were incubated with a polyclonal chicken anti-GFP antibody (Aves Labs, Tigard, OR, United States). Analysis of endothelial E-selectin and CD31 (both antibodies from Abcam, Cambridge, United Kingdom) co-localization was performed on sequential sections. After washing, sections were incubated with a secondary anti-rabbit labeled polymer (EnVision®, Agilent Dako, Barcelona, Spain) for 20 min at 37° C. Finally, immunolabeling was revealed using 3-3′diaminobencidine (DAB) and counterstained with hematoxylin. Positive reaction was identified as a dark-brown precipitated with a membrane or cytoplasmic pattern for CD3, E-selectin, and CD31 or GFP staining, respectively. Histopathologic and immunohistochemical analysis was performed using a standard light microscope (Zeiss Axio A10, Carl Zeiss, Barcelona, Spain).

### Quantification of Cytokines and Nitric Oxide

Murine IFN-γ, IL-1β, TNF-α, TGF-β, IL-1β, IL-12, IL-6, IL-17, PGE₂ and IDO were quantified in plasma of animals or culture supernatants by ELISA (RayBiotech, Peachtree Corners, GA, United States; Diaclone, bioNova Cientifica, Madrid, Spain; Elabscience, Bethesda, MD, United States and Cusabio Biotech, Houston, TX, United States). Human TGFβ, IDO and IL-10 ELISA kits were purchased from RayBiotech and Elabscience. Nitric oxide was detected in culture supernatants using a modified Griess reagent (Parameter™ total nitric oxide and nitrate/nitrite assay, R&D Systems). Briefly, all nitrates are converted into nitrites by nitrate reductase, and total nitrites detected by the Griess reaction (Miranda et al., 2001). Nitrites were assayed spectrophotometrically using a Griess reagent (1% sulfanilamide in 5% phosphoric acid and 0.1% N-1-naphthylethylenediamine dihydrochloride in twice distilled H2O), measuring optical density at 550 nm. Samples and standards were analyzed in triplicates according to the manufacturer's instructions.

### Statistical Analysis

Data are expressed as mean±SD. The number of independent experimental replicates is indicated in figure legends, with n representing the number of replicates for in vitro experiments or number of animals used per experimental group. Comparisons between groups were analyzed using Student's paired t-test or one-way ANOVA followed by Bonferroni's post-hoc comparisons tests. Survival curves were plotted using Kaplan-Meier estimates and statistically analyzed using the Mantel-Cox log-rank test. Linear correlation was determined by using the Pearson correlation coefficient. P values <0.05 were considered statistically significant. GraphPad prism 5.0 was used to perform statistical analyses and to generate graphs.

### Example 2—Results

E-Selectin Expression is Upregulated in Microvessels within Target Tissues of aGvHD

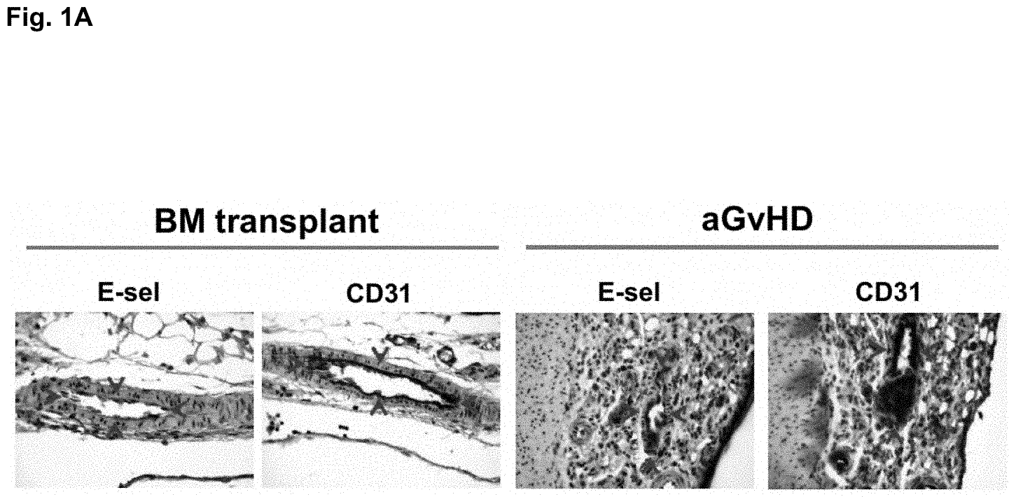

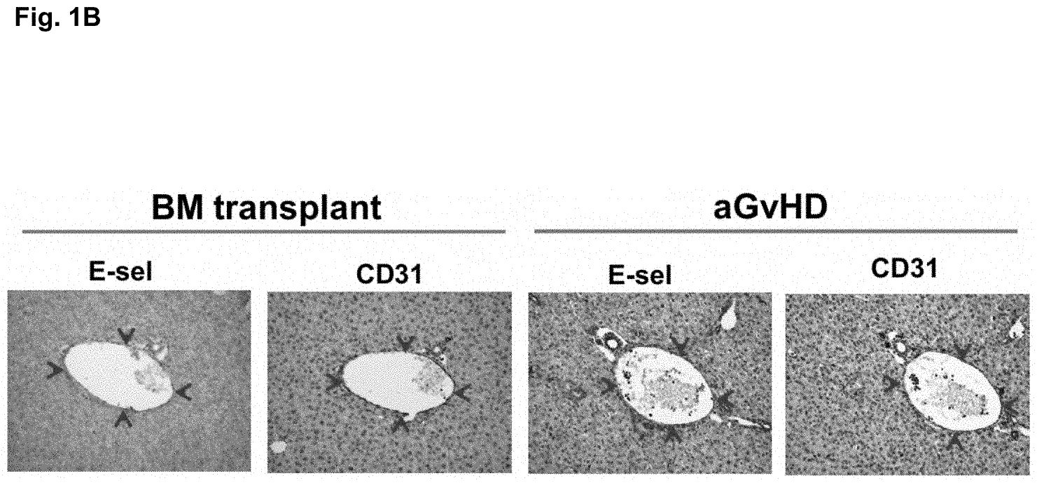

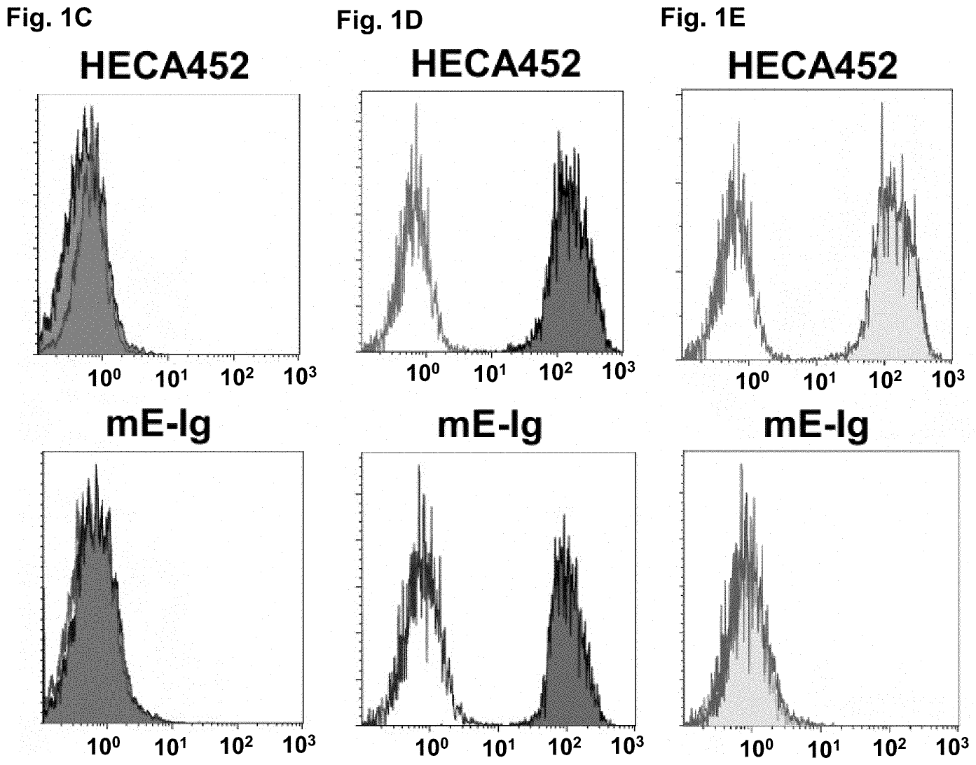

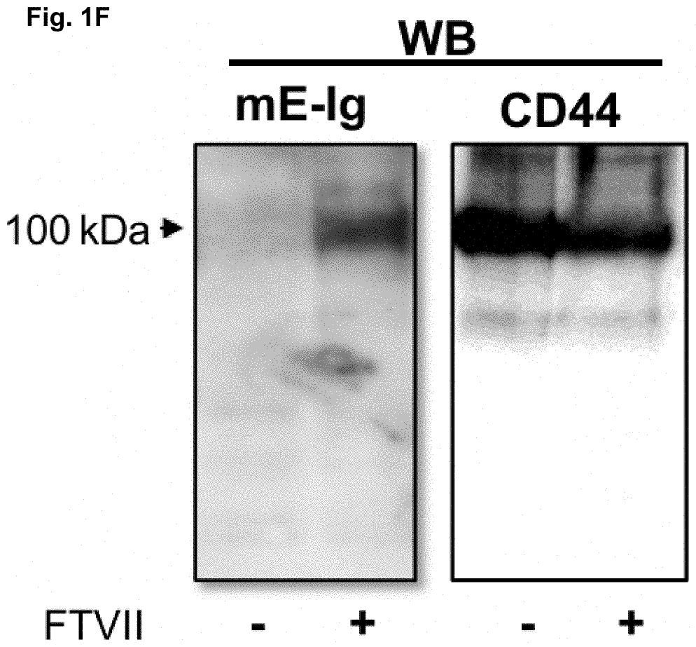

E-selectin expression was assessed by immunohistochemistry in samples of skin, liver and intestine from healthy C57BL/6 mice (H-2^{b}), and from C57BL/6 mice that were transplanted with MHC-mismatched BALB/c (H-2^{d}) bone marrow with donor splenocytes (“allo-HSCT/S” group) or without splenocytes (“allo-HSCT” group). In mice without aGVHD (healthy C57BL/6 mice and those allo-HSCT mice receiving “bone marrow only”, i.e., without addition of donor splenocytes), no significant E-selectin staining was observed in skin, liver or intestinal vessels. However, in allo-HSCT/S mice (all of whom developed florid aGvHD and died within 14 days post-transplant), microvessels in the intestine (FIG. 1A), liver (FIG. 1), and skin consistently displayed E-selectin, which co-localized with the endothelial marker CD31 in sequential sections. Notably, the intensity of E-selectin expression in gut and liver was uniformly higher than that in skin.

FTVII Treatment Enforces HCELL Expression on mAdMSCs

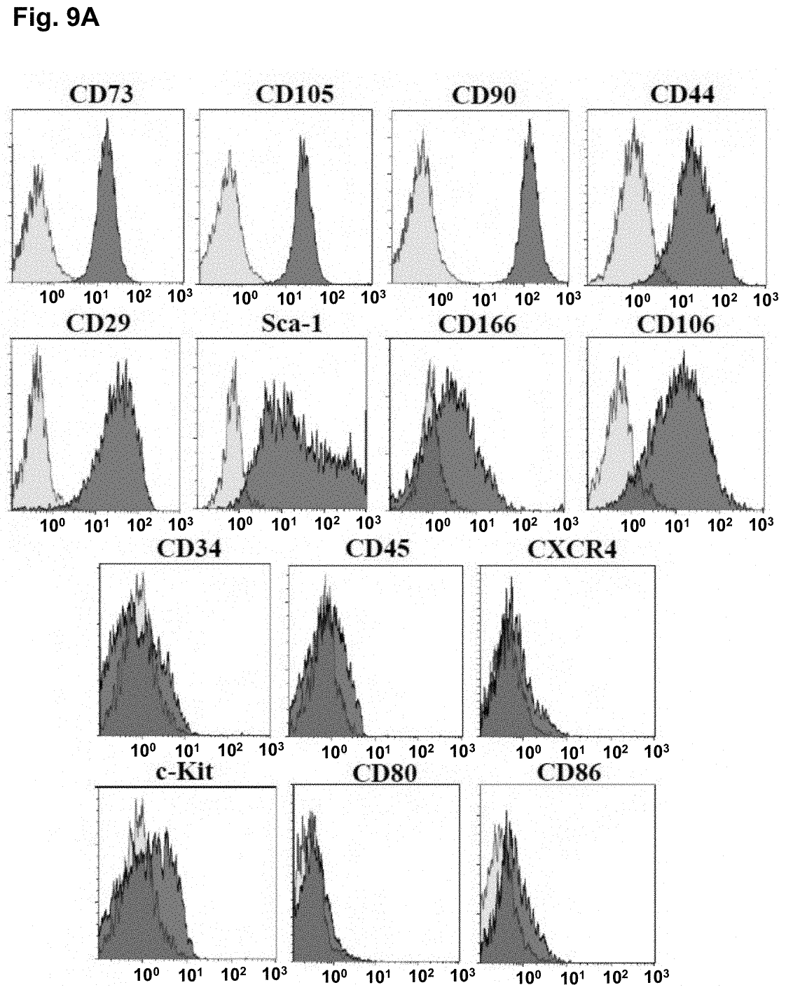

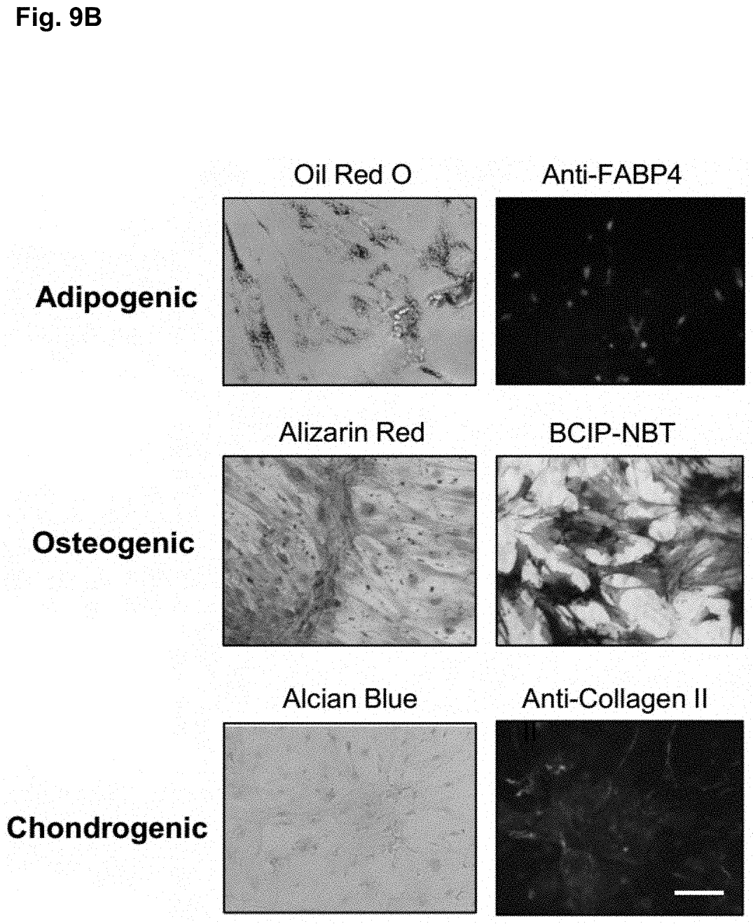

Culture-expanded mAdMSCs displayed characteristic MSC immunophenotype (FIG. 9A) and multipotential differentiation to adipocytes, osteoblasts and chondroblasts (FIG. 9B), and did not natively stain with the anti-sLe^{x} monoclonal antibody HECA452 nor bound to murine E-selectin/Human IgG1-Fc chimera (“E-selectin-Ig chimera”; mE-Ig), a reporter for E-selectin ligand activity (FIG. 1C). Exofucosylation of mAdMSCs using fucosyltransferase VII (FTVII) engendered robust sLe^{x} expression and commensurate Ca²⁺-dependent mE-Ig binding (FIG. 1D), providing direct evidence of creation of E-selectin ligands. Digestion of exofucosylated mAdMSCs (“FucmAdMSCs”) with broad-spectrum proteases such as proteinase K and bromelain significantly reduced mE-Ig binding but not staining by mAb HECA452, indicating that glycoproteins instead of glycolipids serve as the main E-selectin ligands (FIG. 1E). Moreover, western blot analysis of FucmAdMSC lysates showed that the principal membrane glycoprotein recognized by mE-Ig is the standard form of CD44, as found previously in exofucosylated marrow-derived murine MSCs (FIG. 1F) (Abdi et al., 2015). Thus, FTVII-mediated exofucosylation enforces HCELL expression on mAdMSCs.

HCELL⁺ mAdMSC Colonize Tissues Affected by aGVHD but not Lymphoid Organs

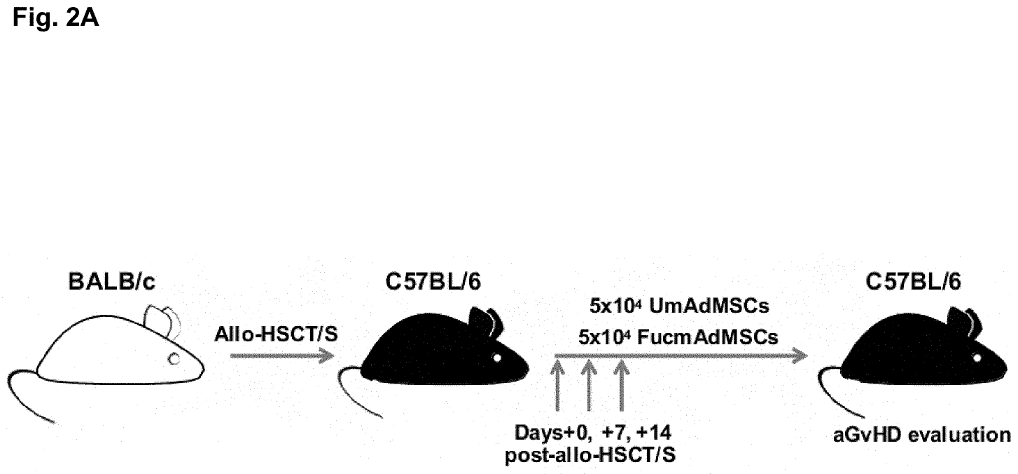

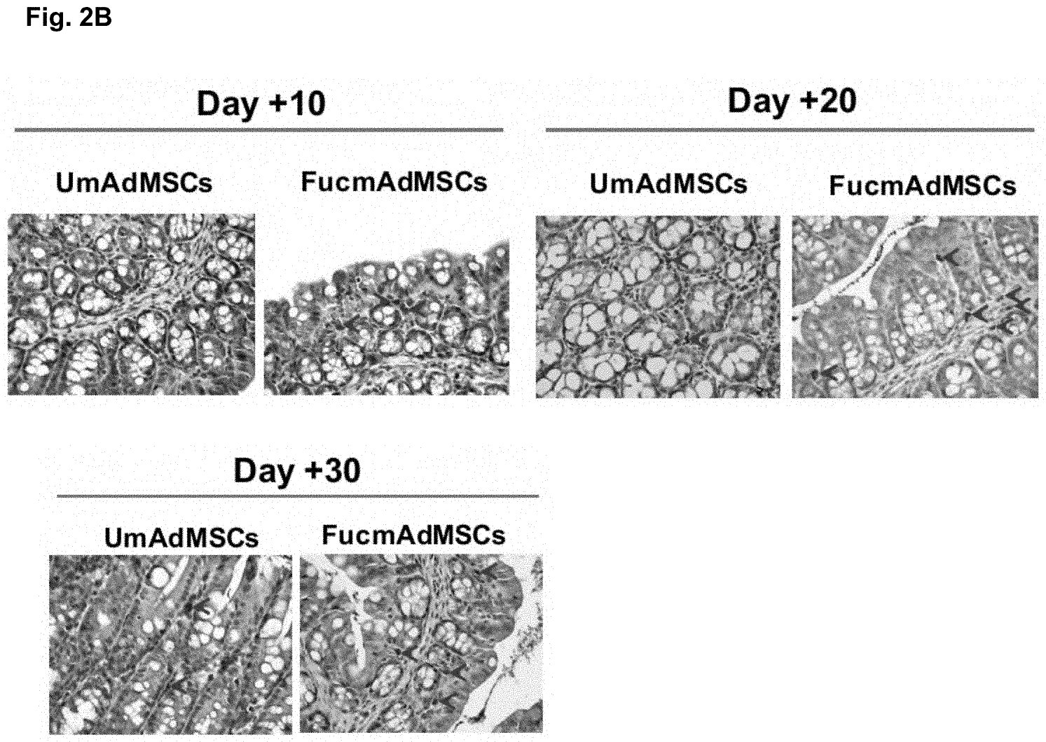

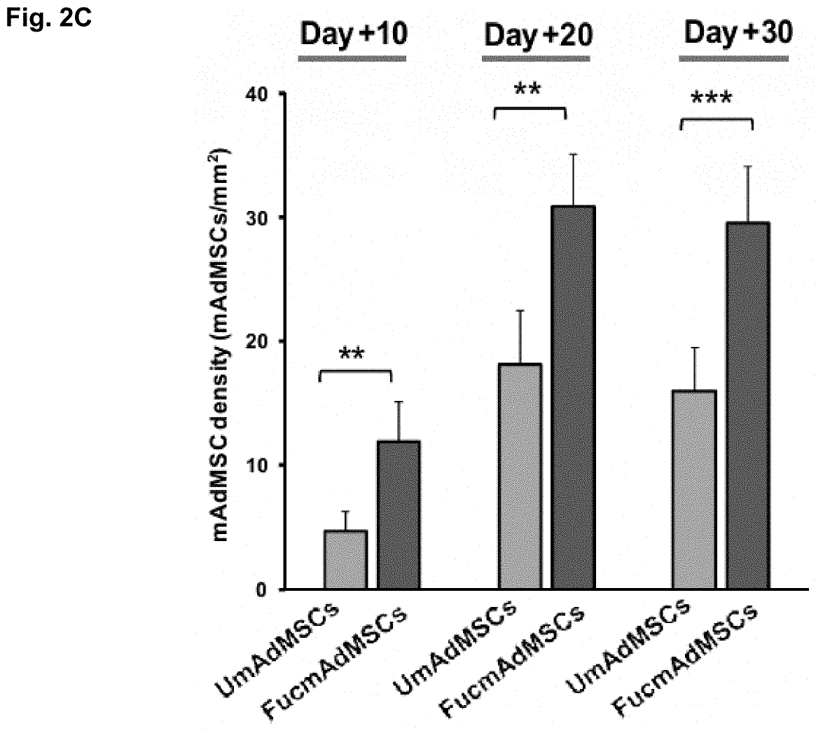

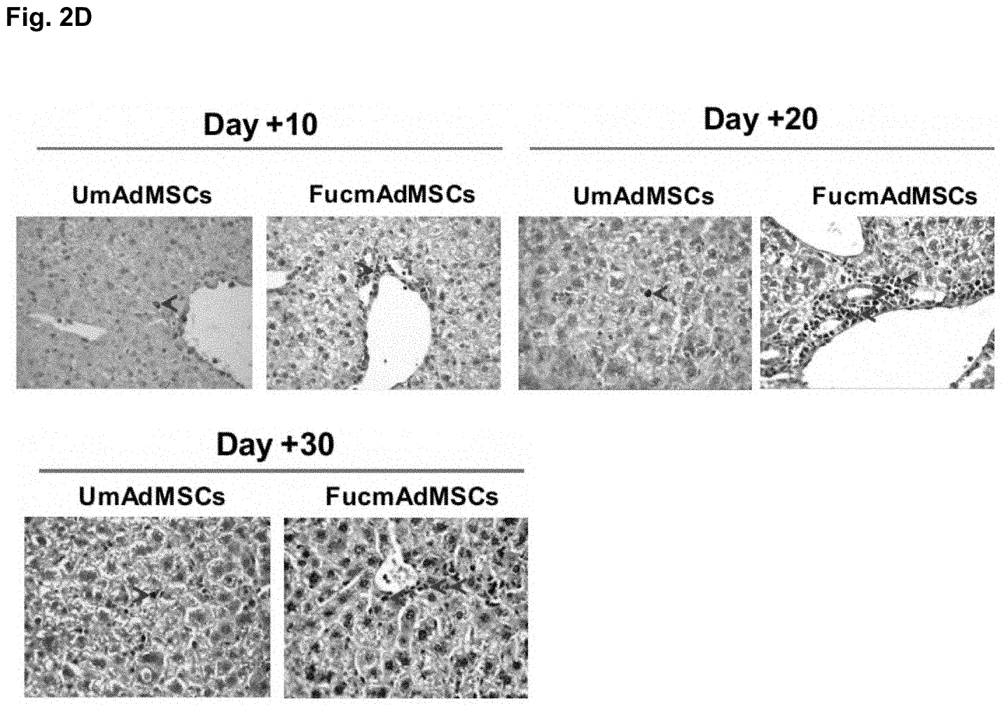

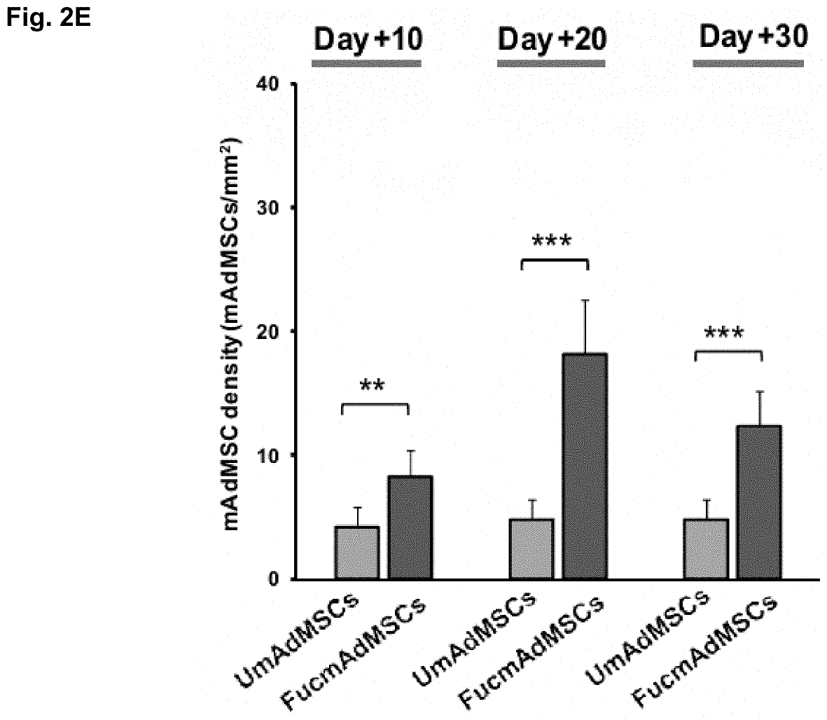

To analyze the effect of enforced HCELL expression on mAdMSC tissue colonization, recipient-type GFP⁺ mAdMSCs was utilized to track parenchymal distribution following systemic administration. To this end, allo-HSCT/S mice received 5×10⁴ GFP⁺ mAdMSCs, either exofucosylated (FucmAdMSCs) or not (UmAdMSCs), by intravenous injection on days 0, +7, and +14 post-transplant. GFP⁺ mAdMSCs were then identified by immunohistochemistry within mesenteric and peripheral lymph nodes, spleen, skin, liver, and gut of recipient mice (schematic diagram shown in FIG. 2A). FucmAdMSCs and UmAdMSCs were not detectable in any lymphoid tissues or in skin at any time-point post-transplantation. However, as early as day 10 post-HSCT (day +10), exofucosylated mAdMSC showed marked intestinal tropism, with FucmAdMSC infiltrates in intestinal lamina propria being three-fold higher than that of mice receiving UmAdMSCs. Intestinal infiltrates steadily increased, plateauing at day +20 post-HSCT (FIGS. 2B and 2C). Similarly, histological analysis of livers obtained at days +10, +20 and +30 from animals treated with FucmAdMSCs showed significantly higher numbers of GFP⁺ cells in hepatic periportal areas compared to that of mice receiving UmAdMSCs (FIGS. 2D and 2E).

Increased mAdMSC Tissue Colonization Improves Survival, Reduces Both Clinical and Histopathologic Severity of aGVHD, and Significantly Decreases Donor T Cell and Neutrophil Infiltration in aGvHD Target Organs

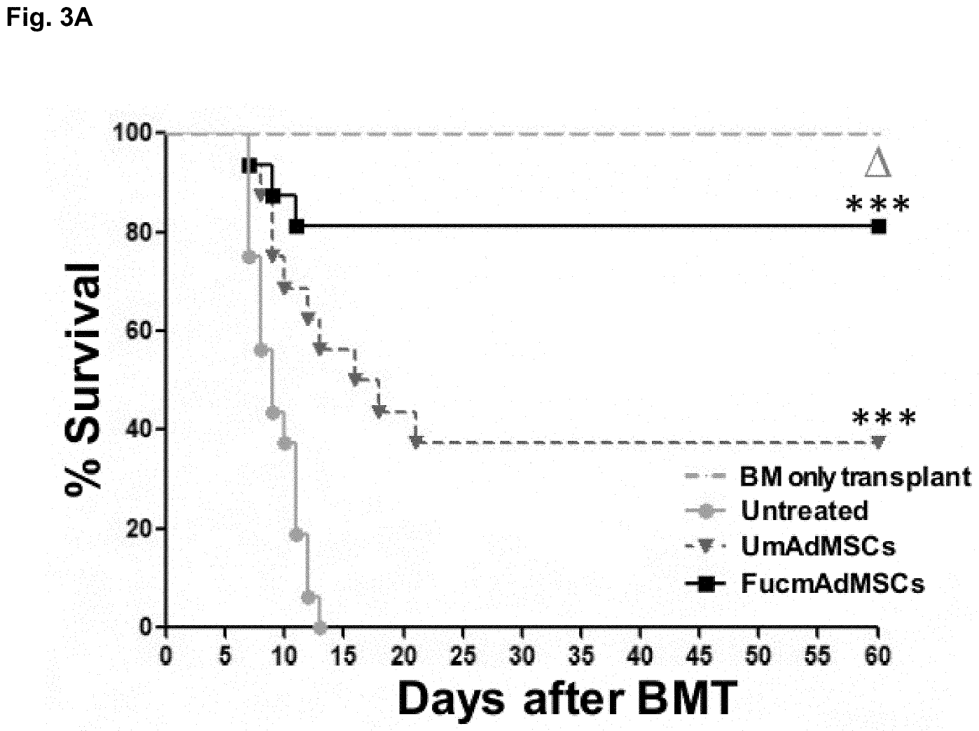

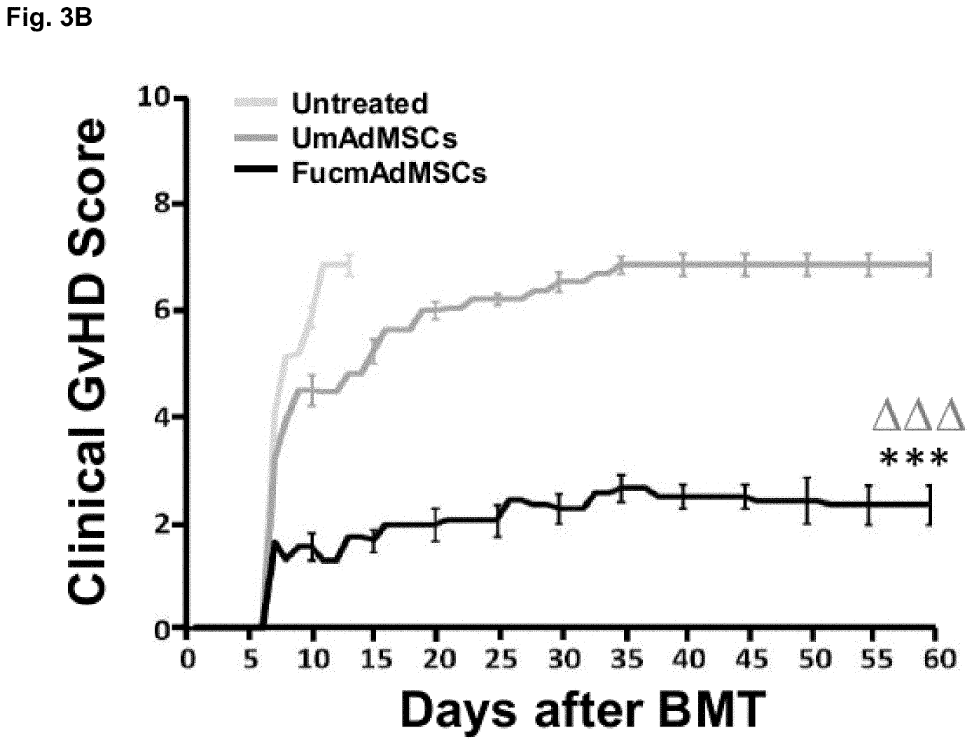

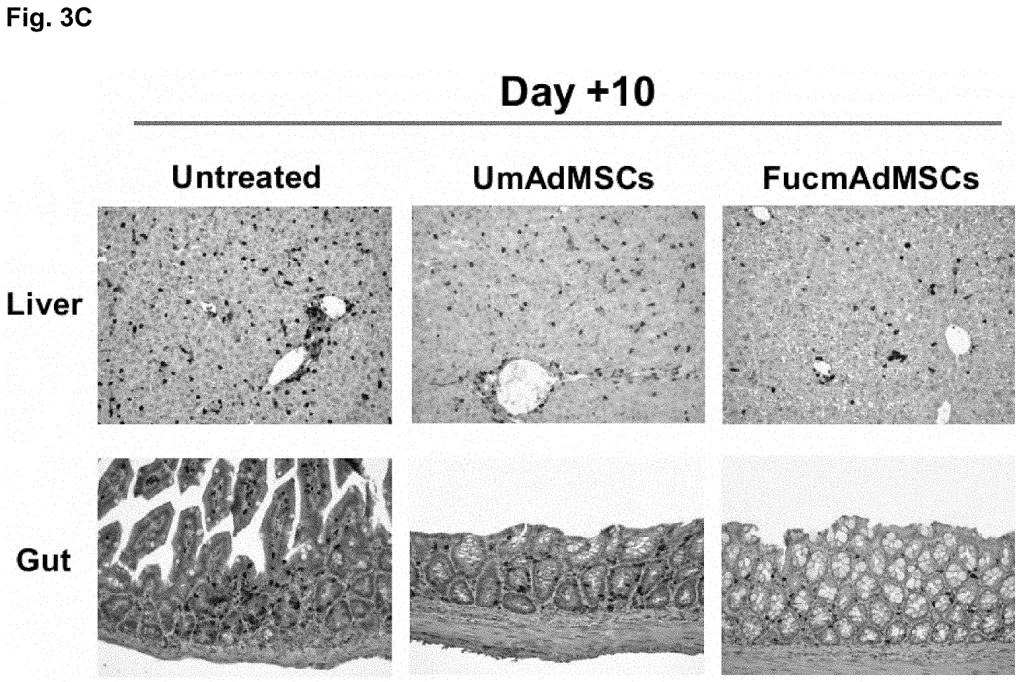

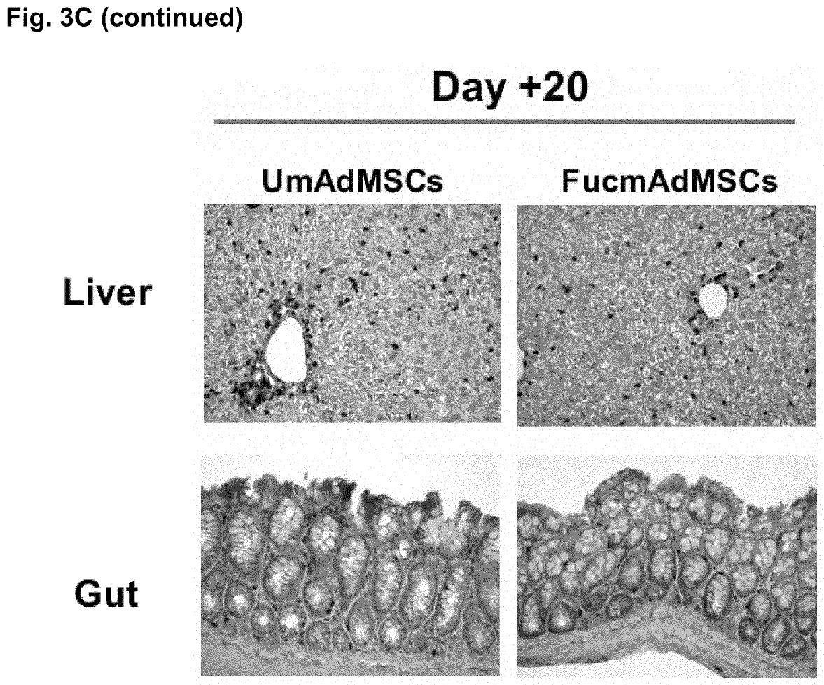

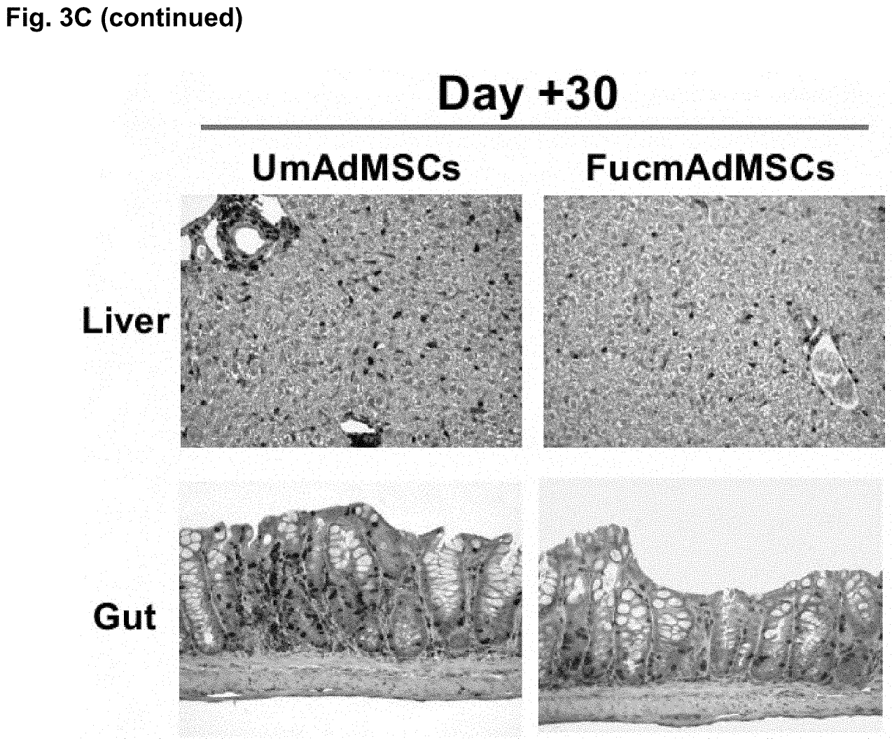

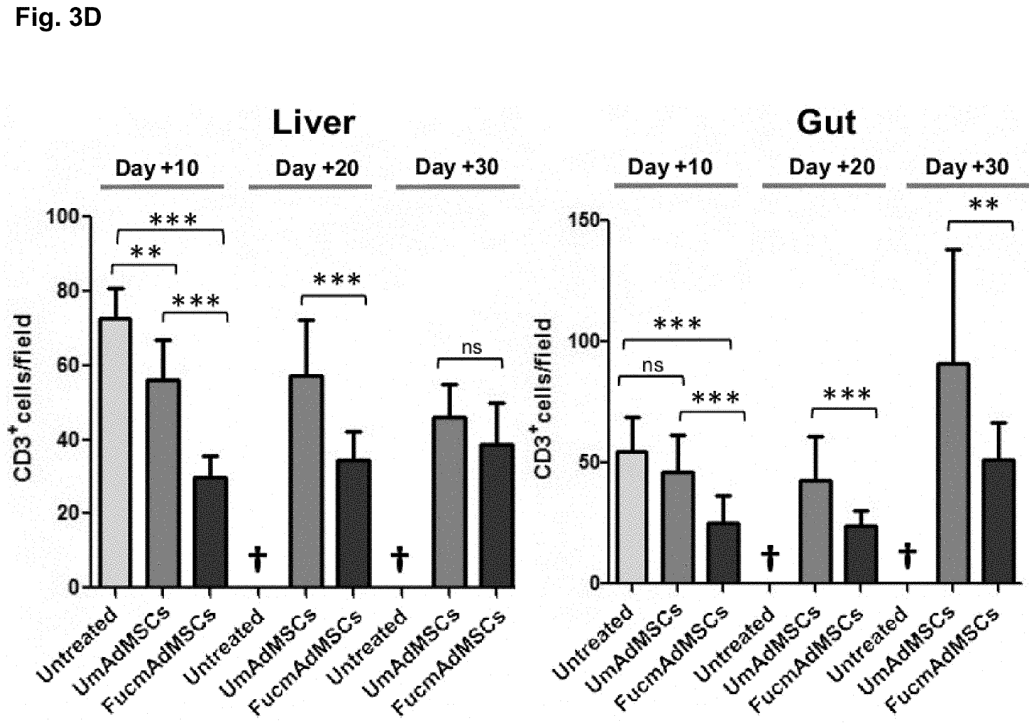

To assess whether enhanced mAdMSC tissue colonization impacts immune-mediated tissue damage, clinical outcome and tissue histology were evaluated in animals undergoing allo-HSCT and allo-HSCT/S, without or with intravenous infusions of 5×10⁴ recipient-type UmAdMSC or FucmAdMSCs on days 0, +7, and +14 post-transplantation. Mice in the allo-HSCT group (i.e., “BMT only” group) did not develop aGvHD, whereas mice receiving allo-HSCT/S without mAdMSC administration (“Untreated” group) displayed a rapid development of florid aGvHD, resulting in death in all mice within 14 days post-transplant (FIG. 3A). Allo-HSCT/S mice that received early post-transplant infusions of UmAdMSCs displayed significantly decreased aGvHD-related mortality compared to mice not receiving mAdMSCs (p<0.001), but infusions of FucmAdMSCs yielded a profound survival advantage compared to those receiving UmAdMSCs (82% versus 38% survival, p<0.05). As shown in FIG. 3B, the marked improved survival of the FucmAdMSC-treated group of animals correlated with a sustained reduction of the clinical aGvHD score, being significantly lower than that observed for mice receiving UmAdMSCs or no mAdMSCs (p<0.001).

Histopathology of inflammatory changes within aGVHD target organs was analyzed beginning at day +10. Whereas no observable lesions were noted in the skin of mice at early times post-transplantation, histological analysis of liver and gut revealed significant differences in the severity of aGvHD between the treatment groups. Livers of Untreated and of UmAdMSC-treated animals showed extensive epithelial damage with abundant periportal inflammatory infiltrates and destruction of the majority of bile ducts (grade III-IV aGvHD), whereas FucmAdMSC-treated mice showed only minimal hepatic injury (grade I-II) (Table 1, showing histopathological scoring of aGvHD in tissues of allo-HSCT/S recipients: Untreated, UmAdMSC-treated or FucmAdMSC-treated mice.).

TABLE 1
GvHD pathological score

| | | | |
|---|---|---|---|
| Group | Skin | Liver | Gut |

Day +10 post-transplantation

| | | | |
|---|---|---|---|
| Untreated | Grade 0-I | Grade III-IV | Grade III |
| UmAdMSCs | Grade 0 | Grade III-IV | Grade III |
| FucmAdMSCs | Grade 0 | Grade I-II | Grade I-II |

Day +20 post-transplantation

| | | | |
|---|---|---|---|
| UmAdMSCs | Grade 0-I | Grade III-IV | Grade II-III |
| FucmAdMSCs | Grade 0-I | Grade I-II | Grade I |

Day +30 post-transplantation

| | | | |
|---|---|---|---|
| UmAdMSCs | Grade 0-I | Grade III-IV | Grade III |
| FucmAdMSCs | Grade 0-I | Grade I-II | Grade I |

Moreover, small intestines of Untreated mice or of mice that received UmAdMSCs similarly displayed focal mucosal ulcerations with moderate villous atrophy (grade III), whereas small intestines of mice receiving FucmAdMSCs had only scattered individual apoptotic cells and limited villous atrophy (grade I-II). Significantly, at later times post-transplantation (days +20 and +30), mice receiving FucmAdMSCs showed reduced aGvHD-associated damage in both liver and small intestine compared to their UmAdMSC-treated counterparts (Table 1).

Given the reported correlation between the level of T cell infiltration and the lesional spectrum of aGvHD-affected organs (Beilhack et al., 2005; Wysocki et al., 2005), levels of CD3⁺ T cells within skin, liver and gut of surviving animals at different time-points after allo-HSCT/S was analyzed. Livers of mice that did not receive mAdMSCs displayed abundant and extensive areas of inflammatory T cell infiltrates within hepatic portal triads at day +10, which were significantly higher than those observed in both mAdMSC-treated groups (p<0.001), with lowest levels observed in FucmAdMSC-treated mice (FIGS. 3C and 3D). Remarkably, after 10-20 days post-HSCT, CD3⁺ T cell counts were significantly lower in livers from animals receiving FucmAdMSCs compared to the UmAdMSC-treated group (p<0.001). At longer times post-HSCT (day +30), the T cell burden in livers of surviving UmAdMSC-treated animals significantly decreased, showing levels similar to that observed in the FucmAdMSC-treated group. The same trend of decreasing numbers of infiltrating T cells was also observed in the intestinal lamina propria of surviving mice at days +10 and +20, although UmAdMSC-treated mice displayed a consistently higher number of T cell infiltrates in gut compared to that of FucmAdMSC-treated mice (p<0.01). Interestingly, however, intestinal T cell infiltrates increased between day +20 and day +30 in the mAdMSC-treated mice to levels higher than at days +10 and +20, but infiltrates were consistently less in FucmAdMSC-treated mice compared to UmAdMSC counterparts (FIGS. 3C and 3D).

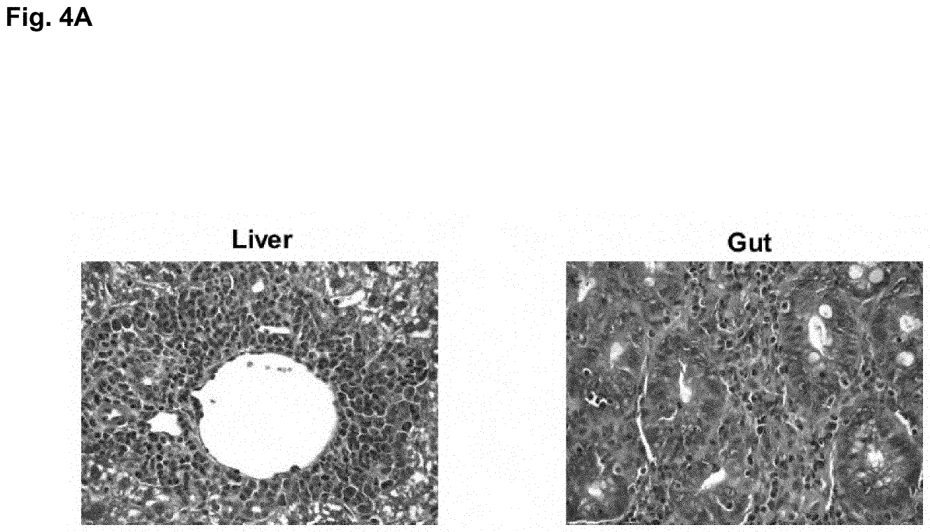

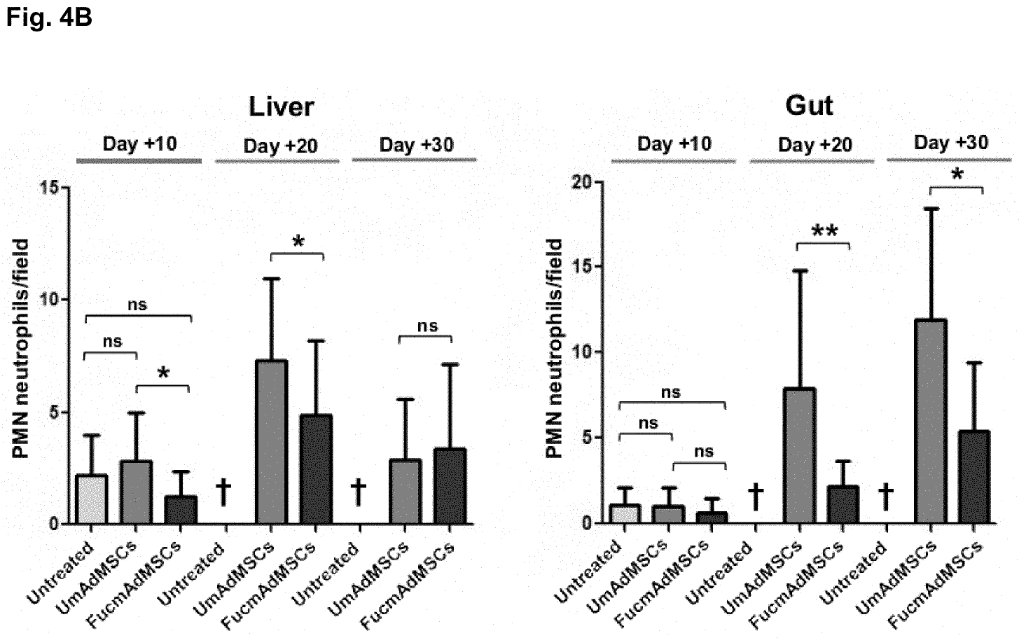

In addition to levels of T cell infiltrates, the presence of neutrophils in aGvHD-affected organs has been described as a highly predictive marker of transplant-related mortality (Socié et al., 2004). Thus, neutrophil infiltrates in liver and gut in surviving mice that received allo-HSCT/S was analyzed. At day +10, there were scarce neutrophil infiltrates in liver and gut in the Untreated, UmAdMSC-treated and FucmAdMSC-treated mice (FIGS. 4A and 4B), consistent with expected neutropenia (i.e., pre-engraftment). However, at day +20, neutrophil infiltrates were prominent in liver and gut from UmAdMSC-treated animals, whereas neutrophil infiltrates were sparse in the FucmAdMSC-treated mice, especially in the gut (p<0.01). At day +30 compared to day +20, similar to that observed for infiltrating T cells, surviving mice had lower neutrophil infiltrates in liver, but higher infiltrates in gut. However, consistently, mice receiving FucmAdMSCs displayed lower neutrophil numbers in these organs compared to mice treated with UmAdMSCs. There was a close inverse linear correlation between the increased numbers of intestinal mAdMSCs and the lower burden of T cells and neutrophils in gut of animals receiving FucmAdMSCs at days +20 and +30 post-transplant (Pearson correlation coefficients (PCC): −0.574 and −0.897 for T cells and neutrophils, respectively). Importantly, there were no significant differences in peripheral blood leukocyte counts among any of the treatment groups (values x 103/μl, Mean+/−SD for each group: (1) Untreated 1.63+/−0.21 lymphocytes and 0.24+/−0.12 neutrophils; (2) UmAdMSC-treated 1.78+/−0.59 lymphocytes and 0.38+/−0.17 neutrophils; FucmAdMSC-treated 1.97+/−0.80 lymphocytes and 0.30+/−0.22 neutrophils), indicating that the observed variances in levels of lymphocyte and neutrophil tissue infiltrates were not due to variations in hematopoietic engraftment.

FucmAdMSC Administration in Mice with aGVHD Prominently Alters Plasma Levels of Pro-Inflammatory and Anti-Inflammatory Cytokines

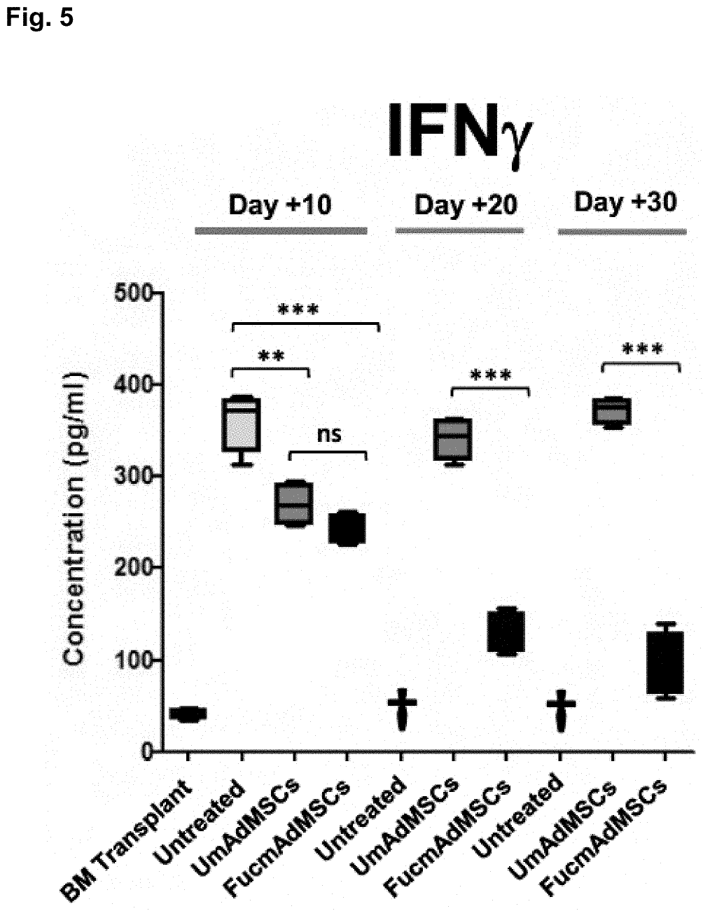

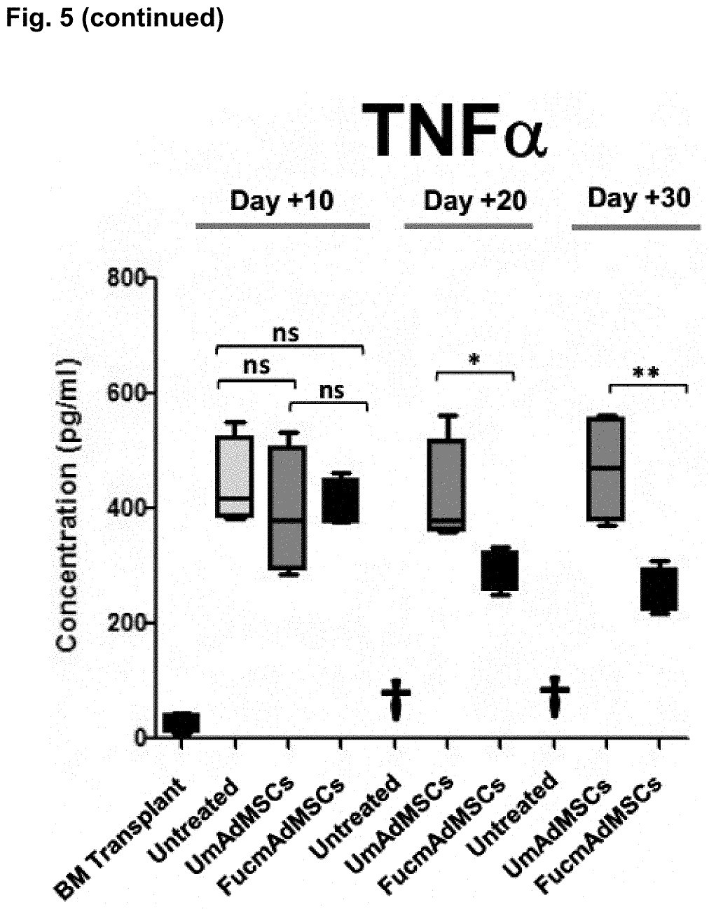

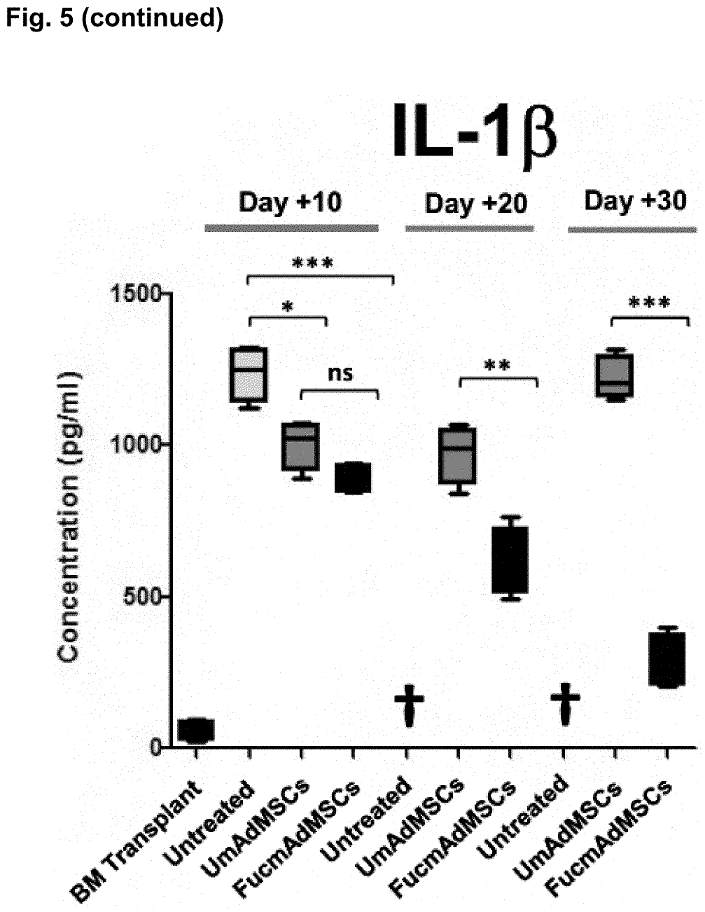

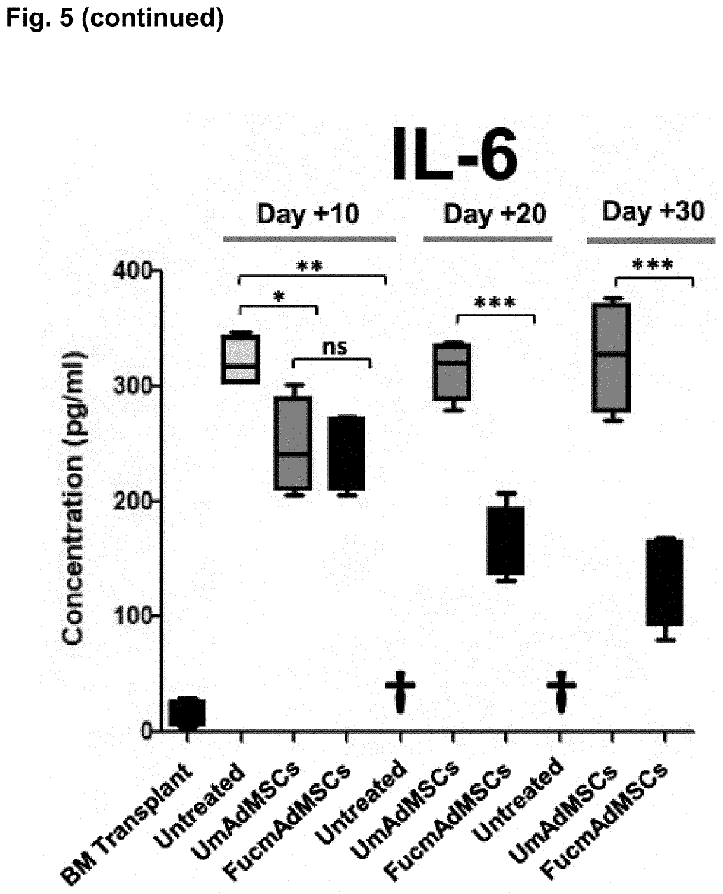

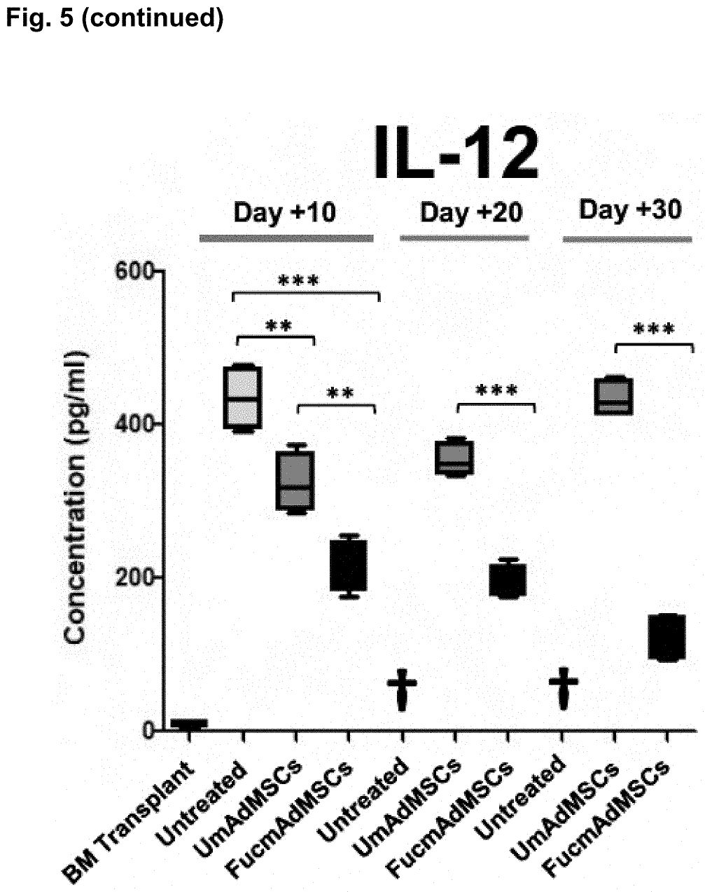

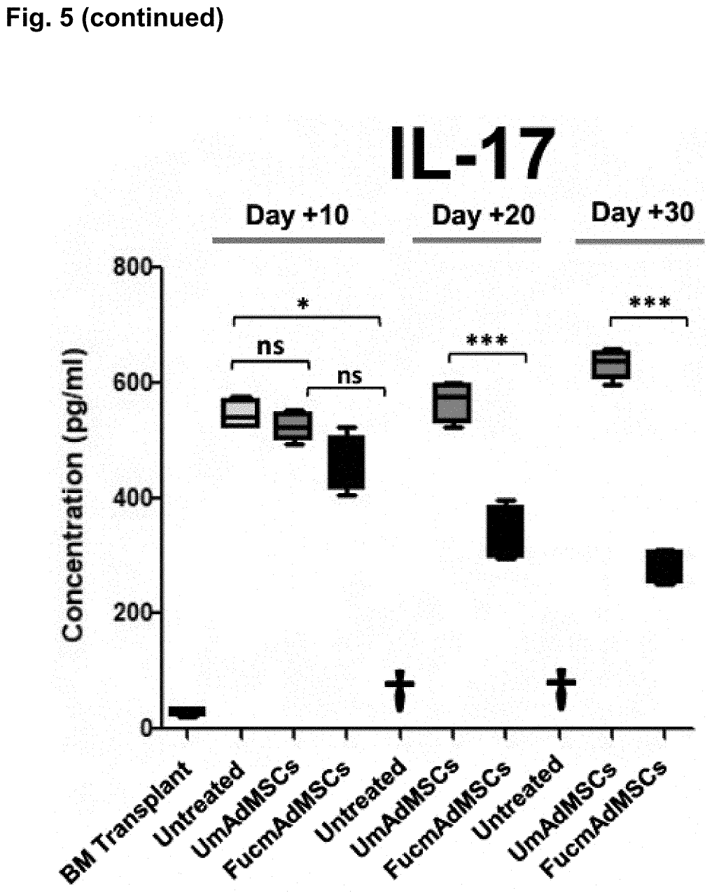

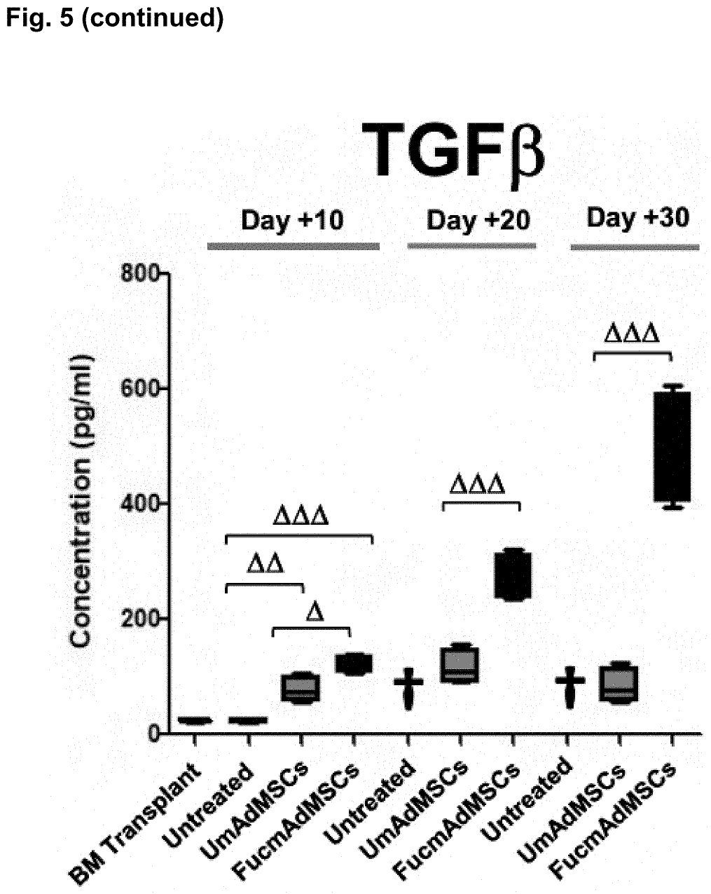

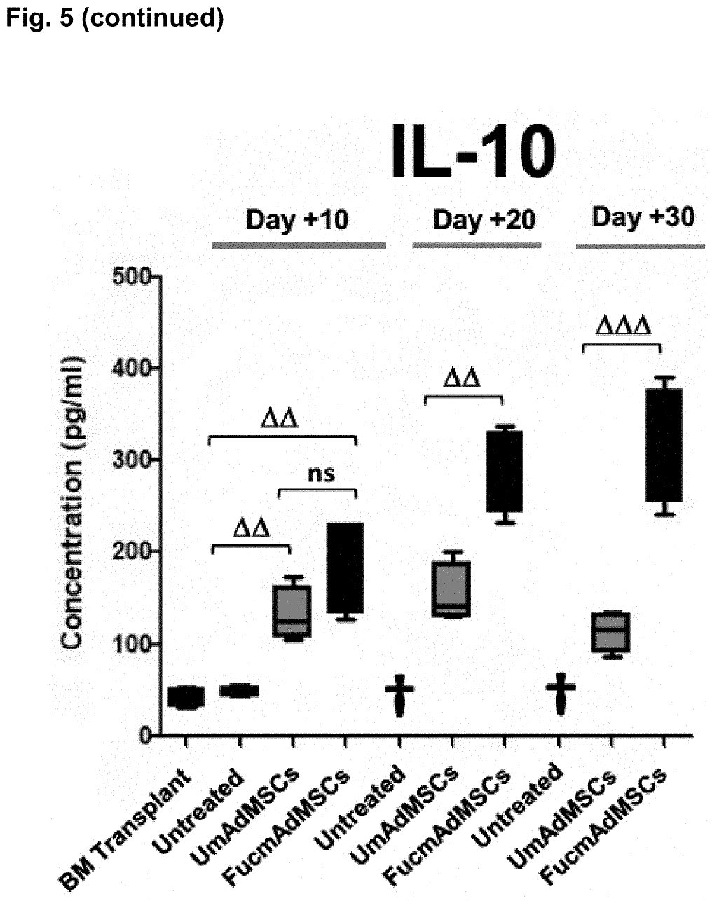

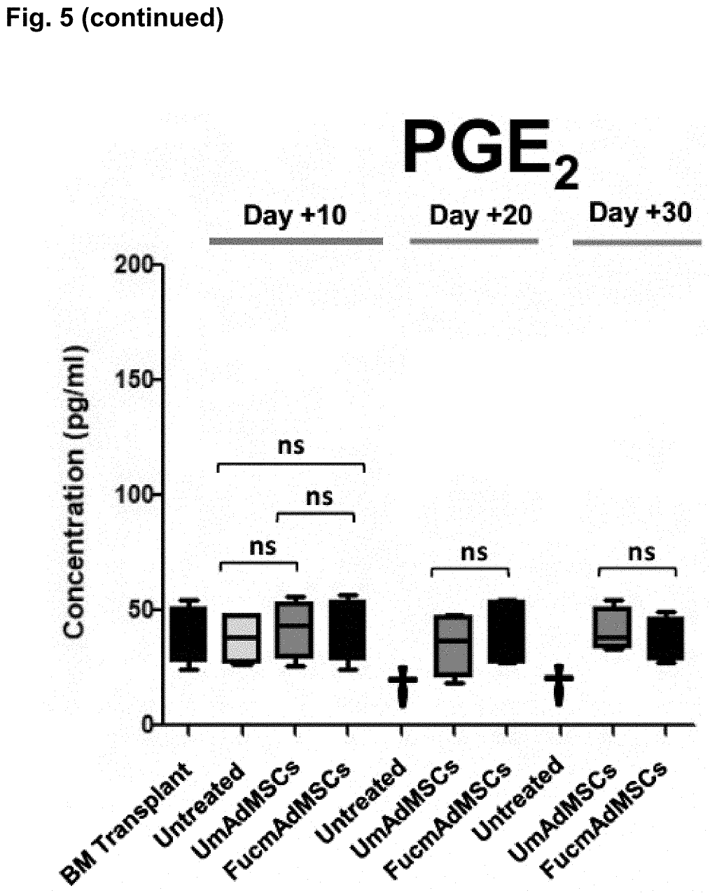

Apart from profound cellular changes within the tissues, and consistent with results of others (Burman et al., 2007; Robb et al., 2011), it was observed that mice receiving allo-HSCT/S without mAdMSC infusion (i.e., Untreated animals) showed marked increases in plasma levels of the pro-inflammatory cytokines IFNγ, TNFα, IL-1β, IL-6, IL-12 and IL-17 on day +10 post-transplantation, and low levels of the anti-inflammatory cytokines TGFβ and IL-10 (FIG. 5). Importantly, administration of UmAdMSCs or FucmAdMSCs yielded a steep decrease in pro-inflammatory mediators compared to that of Untreated mice, with strikingly lower levels of TNFα, IL-1β, IL-6, IL-12, and IL-17 in those mice receiving FucmAdMSCs. Plasma concentrations of PGE₂, an anti-inflammatory factor released by MSCs (Yanez et al., 2010), did not change among any of the treatment groups, but mice receiving mAdMSCs had increased levels of anti-inflammatory cytokines TGFβ and IL-1β, with markedly higher levels of these cytokines in those mice receiving FucmAdMSCs. Thus, as compared to mice receiving UmAdMSCs, FucmAdMSC-treated mice displayed a more persistent and more profound depression of plasma levels of pro-inflammatory cytokines with much higher and more sustained increases in anti-inflammatory cytokines.

HCELL/CD44 Ligation by Either E-Selectin or HA, Respectively, Augments mAdMSC-Induced Inhibition of Mitogen-Stimulated Splenocyte Proliferation and Boosts Production of Immunoregulatory Molecules by Both Murine and Human MSCs

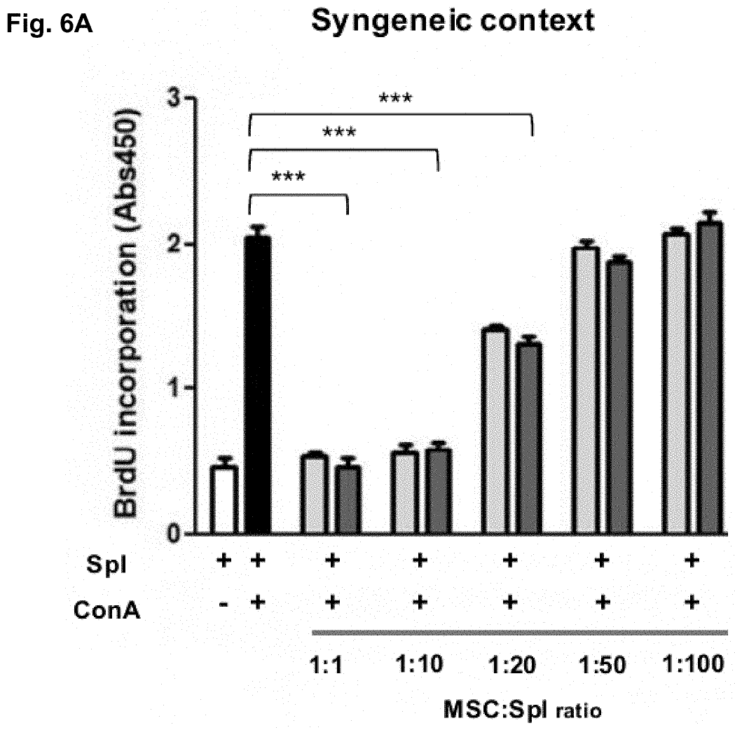

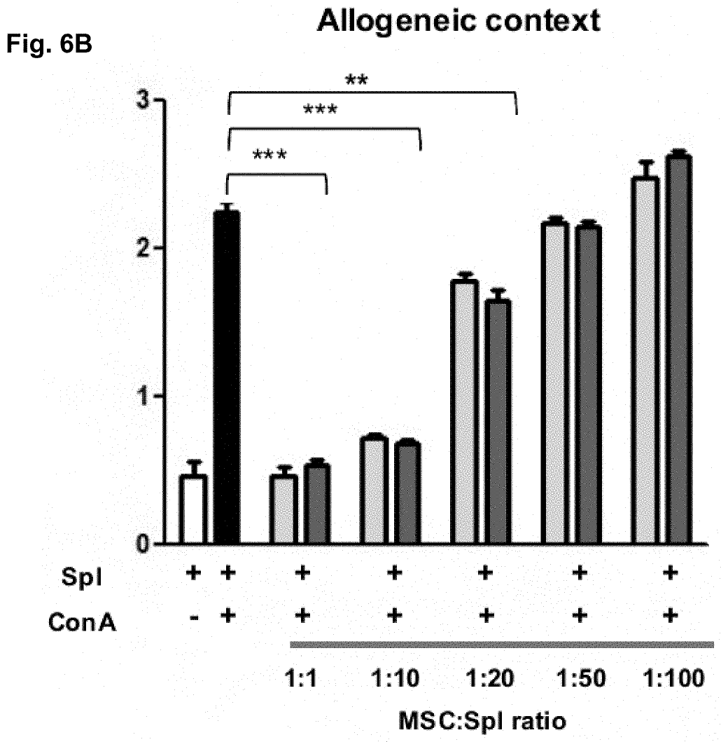

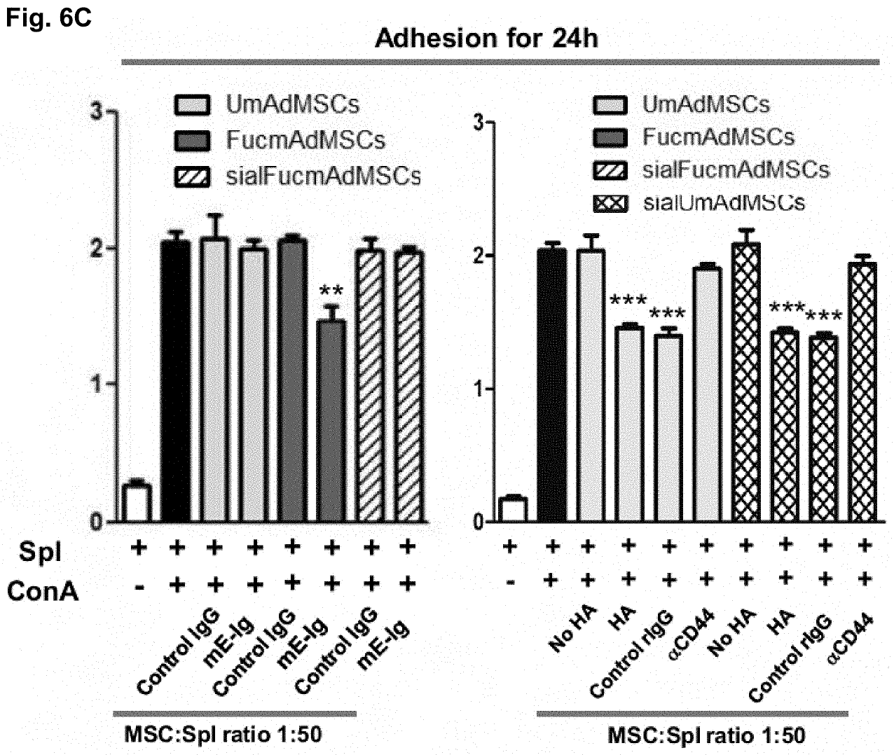

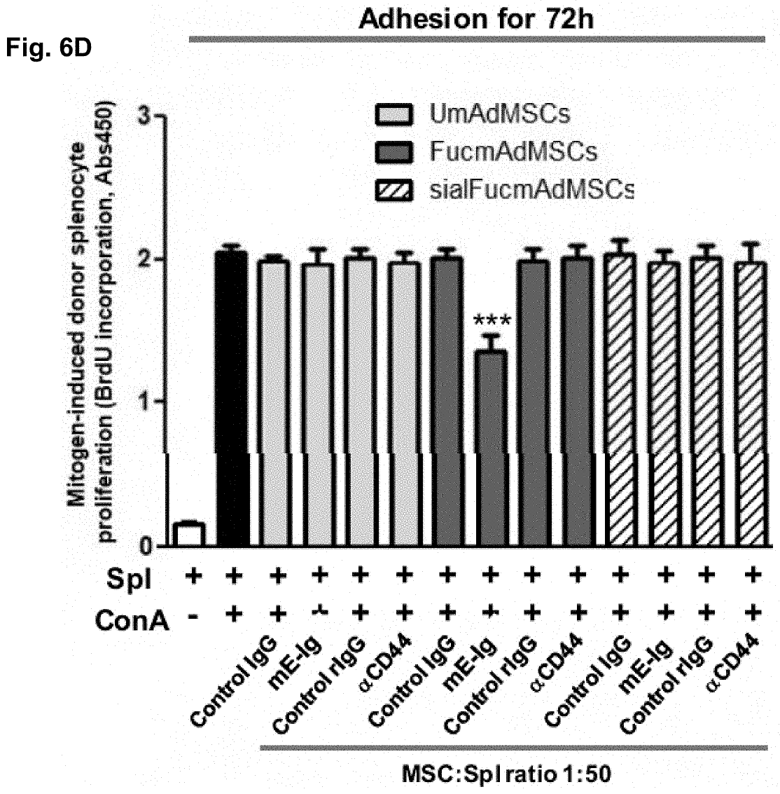

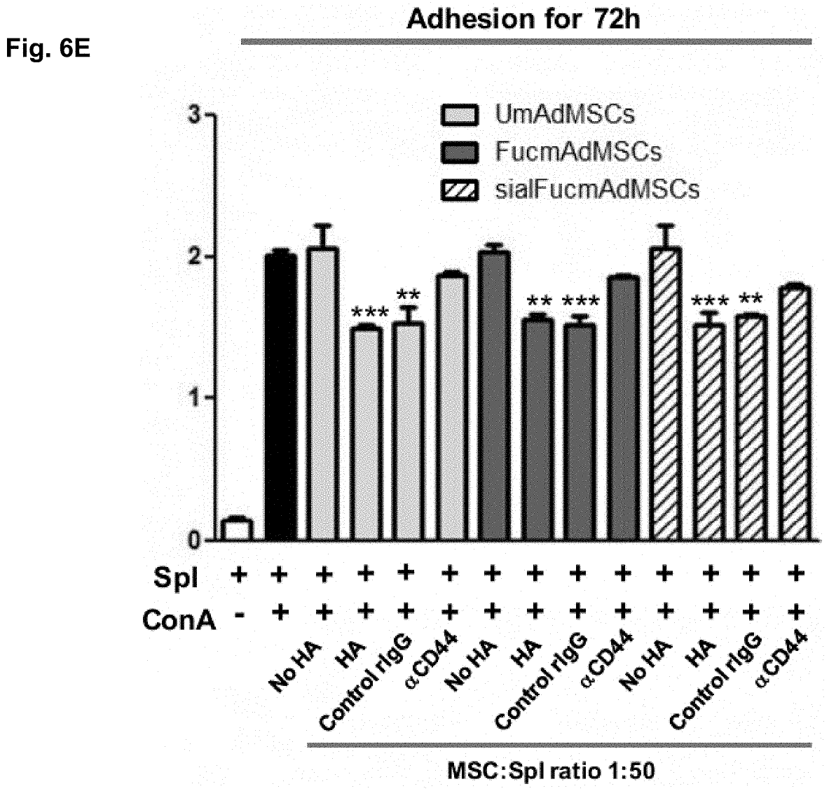

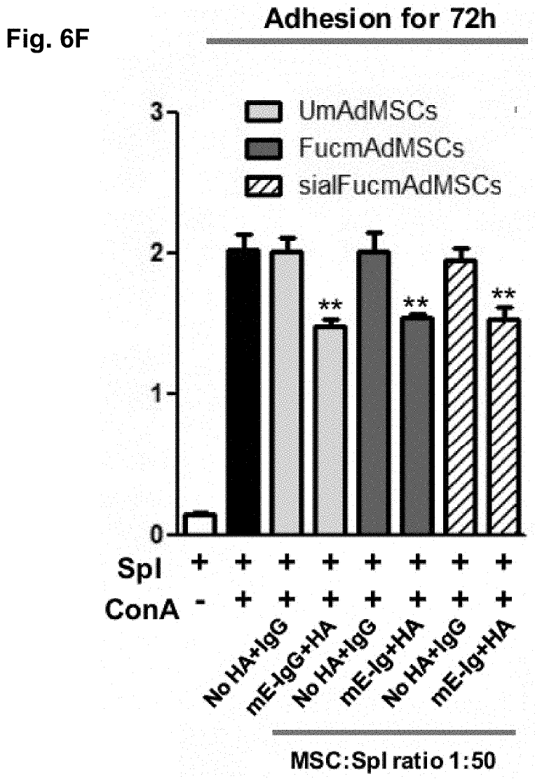

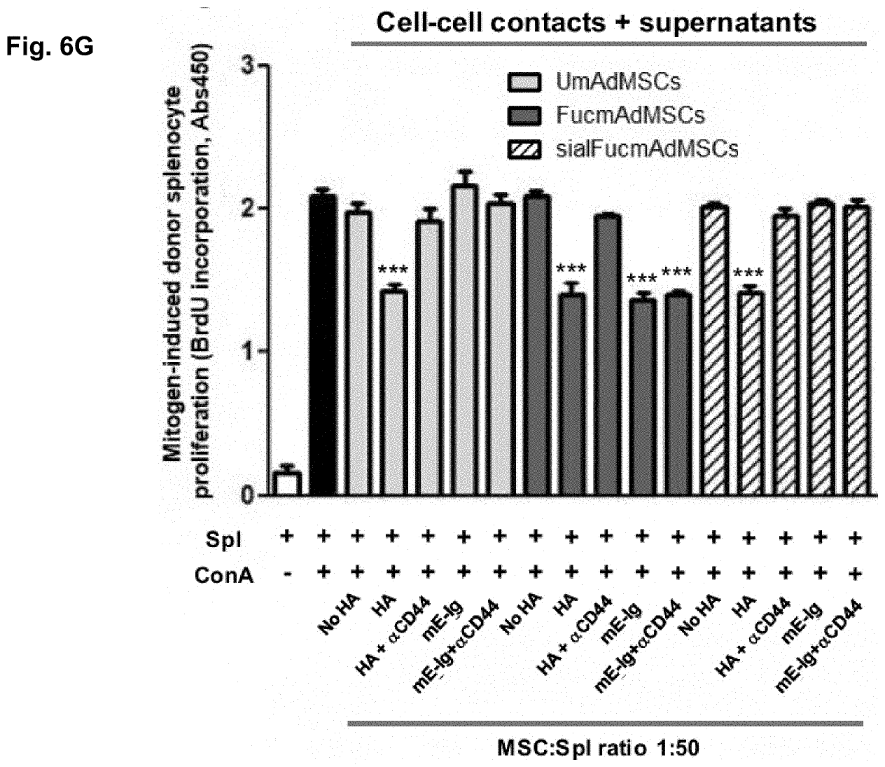

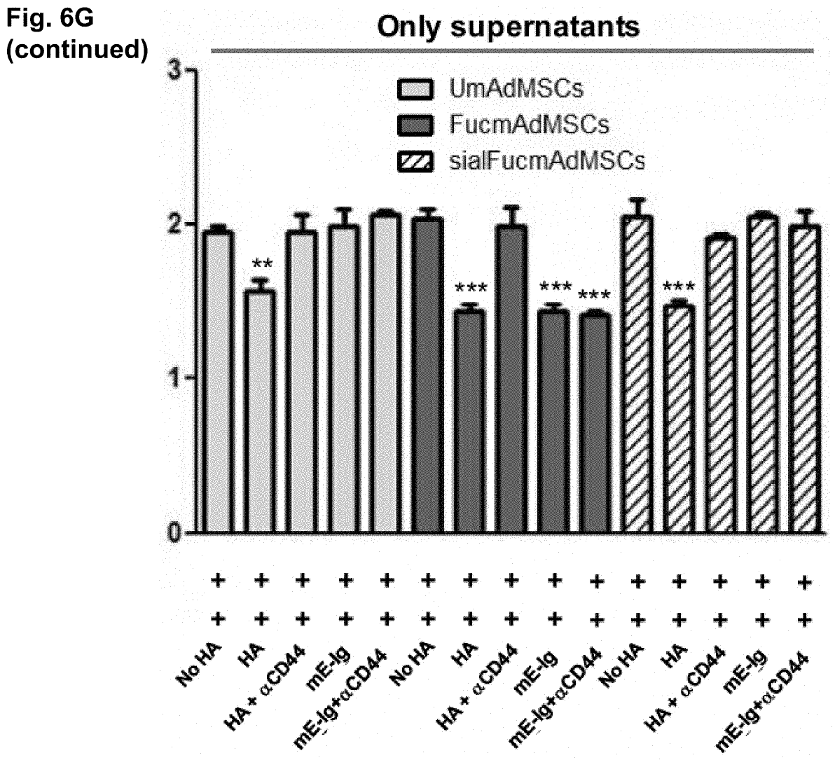

To analyze whether exofucosylation itself impacts the immunoregulatory properties of mAdMSCs, the capacity of UmAdMSCs and FucmAdMSCs to suppress mitogen-induced proliferation of both syngeneic and allogeneic murine T cells was evaluated. To this end, responding splenocytes were stimulated with concanavalin A (ConA) in the presence of varying amounts of both types of mAdMSCs, from ratios of 1:1 to 1:100 MSC:splenocyte. Co-incubation with UmAdMSCs or FucmAdMSCs significantly inhibited mitogen-stimulated splenocyte proliferation in a dose-dependent manner from ratio 1:1 to ratio 1:20, with identical inhibitory effects using both types of mAdMSCs, in both syngeneic (FIG. 6A) and allogeneic contexts (FIG. 6B). These findings indicate that mAdMSC-mediated suppression of mitogen-induced T cell proliferation is unaffected by the exofucosylation process and the resulting fucose installation on CD44.

The observed higher suppressive effect coincident with increased MSC:splenocyte ratios suggests that increased MSC infiltrates in aGvHD-affected tissues could, alone, yield heightened in situ immunomodulation. However, apart from increased MSC infiltration, it was also reasoned that since HCELL⁺ MSCs engage E-selectin during the process of extravasation, HCELL ligation could impact the immunobiology of FucmAdMSCs. Accordingly, in vitro mitogenic assays were performed of mAdMSC/ConA-stimulated splenocyte (“ConA-splenocyte”) co-cultures using mAdMSCs that were incubated with mE-Ig chimera or isotype control human IgG1 for 24 h prior to introduction of splenocytes, and maintained in contact with mE-Ig chimera or IgG1, respectively, during the co-culture period. As shown in FIG. 6C (left), at 1:50 ratio of MSC:ConA-splenocyte, FucmAdMSCs (i.e., HCELL⁺ mAdMSCs) previously incubated for 24 h with mE-Ig, but not with isotype control IgG1, more completely dampened donor T cell proliferation in the mitogenic assay than FucmAdMSCs not previously incubated with mE-Ig. Importantly, this effect was abrogated by elimination of E-selectin adherence by sialidase treatment of FucmAdMSCs (“sialFucmAdMSCs,” which are HCELL⁻), indicating that the observed augmented anti-proliferative effect is directly related to the capacity of HCELL to engage E-selectin. To determine whether this effect is secondary to ligation of CD44 itself, this analysis was performed using UmAdMSCs previously cultured for 24 h in presence of the conventional CD44 ligand, HA, with continued exposure to HA during the co-culture with ConA-splenocytes. As is shown in FIG. 6C (right), ligation of CD44 with HA induced a similar anti-proliferative effect, and, consistent with specificity for engagement of CD44, there was no boosting of UmAdMSC (i.e., HCELL⁻ mAdMSC) immunomodulation in presence of mE-Ig.

To further evaluate the effects of E-selectin-mediated HCELL ligation or HA-mediated CD44 ligation in MSC immunobiology, in vitro mitogenic assays were performed using ConA-splenocytes and HCELL⁺- or HCELL⁻ mAdMSCs that were previously incubated for 72 h in presence or absence of input E-selectin and HA (FIGS. 6D-6F). Extending the E-selectin or HA pre-incubation time from 24 h to 72 h did not further potentiate the relevant MSC-mediated anti-proliferative effect. However, whereas UmAdMSCs or sialFucmAdMSCs (each being HCELL⁻ mAdMSCs) displayed an improved anti-mitogenic effect only after CD44-mediated HA ligation (an effect that was abrogated in presence of a blocking anti-mouse CD44 antibody) (FIG. 6E), FucmAdMSCs exhibited a substantial anti-mitogenic effect after either HA or E-selectin engagement that was abrogated by function-blocking anti-CD44 mAb treatment or sialidase treatment, respectively (FIGS. 6D and 6E); the fact that HA exposure of MSCs equally enhanced the anti-mitogenic effect of FucmAdMSCs and UmAdMSCs indicates that the creation of HCELL by exofucosylation does not affect the capacity of CD44 to bind HA, and, thus, HCELL engagement of either HA or E-selectin potentiates MSC immunomodulation. Importantly, for either UmAdMSCs or FucmAdMSCs, the simultaneous pre-incubation with both ligands (i.e., mE-Ig and HA) did not additively augment the MSC immunomodulatory effect on lymphocyte proliferation (FIG. 6F), and, moreover, sialidase-treated FucmAdMSCs (sialFucmAdMSCs) and sialidase-treated UmAdMSCs (sialUmAdMSCs) did not show an increased anti-mitogenic effect after HA-mediated ligation of CD44 compared to HA-ligation of FucmAdMSCs or UmAdMSCs not treated with sialidase (FIG. 6C, right). This latter result indicates that, in contrast to reported findings in other cell types (Skelton et al., 1998), the native sialylation of CD44 (on terminal type 2 lactosamines) of MSCs does not inhibit its binding to HA. Altogether, these results indicate that ligation of the CD44 protein, whether as HCELL via E-selectin or CD44/HCELL via HA, unleashes MSC anti-mitogenic effects.

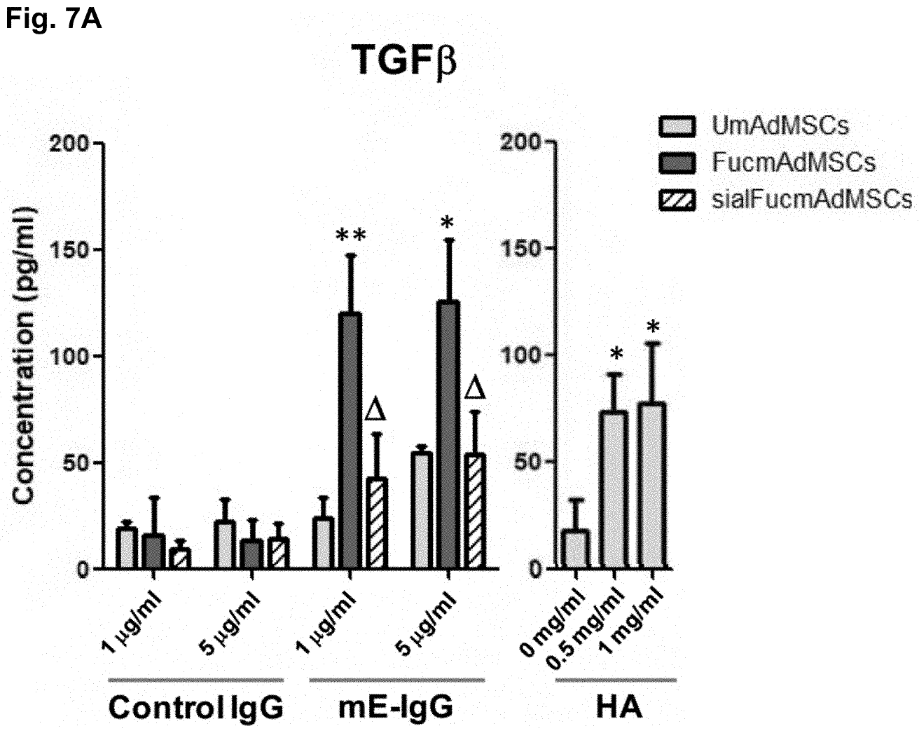

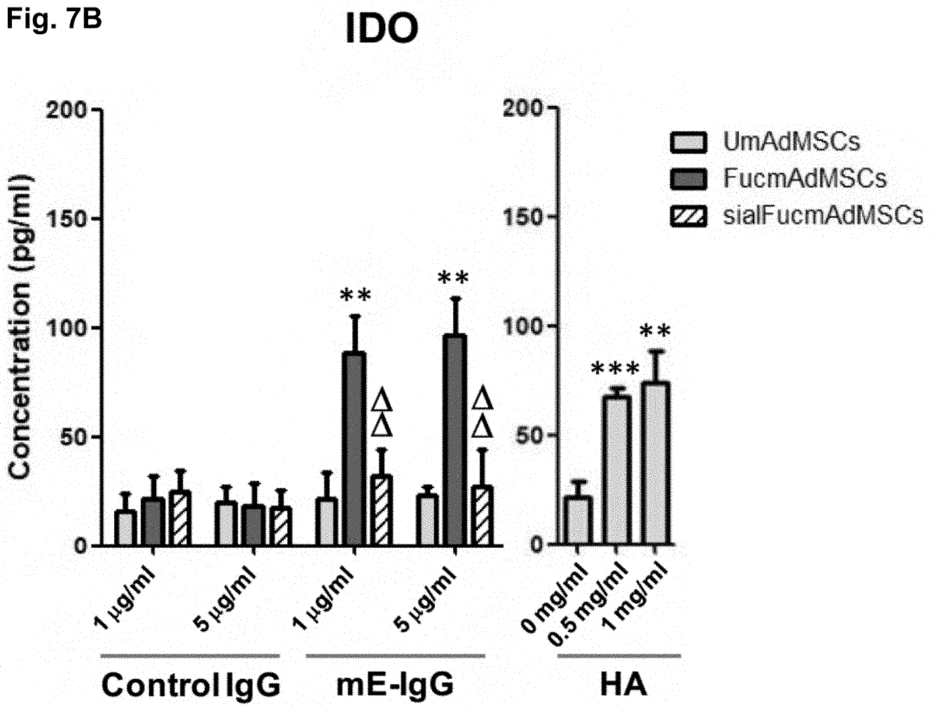

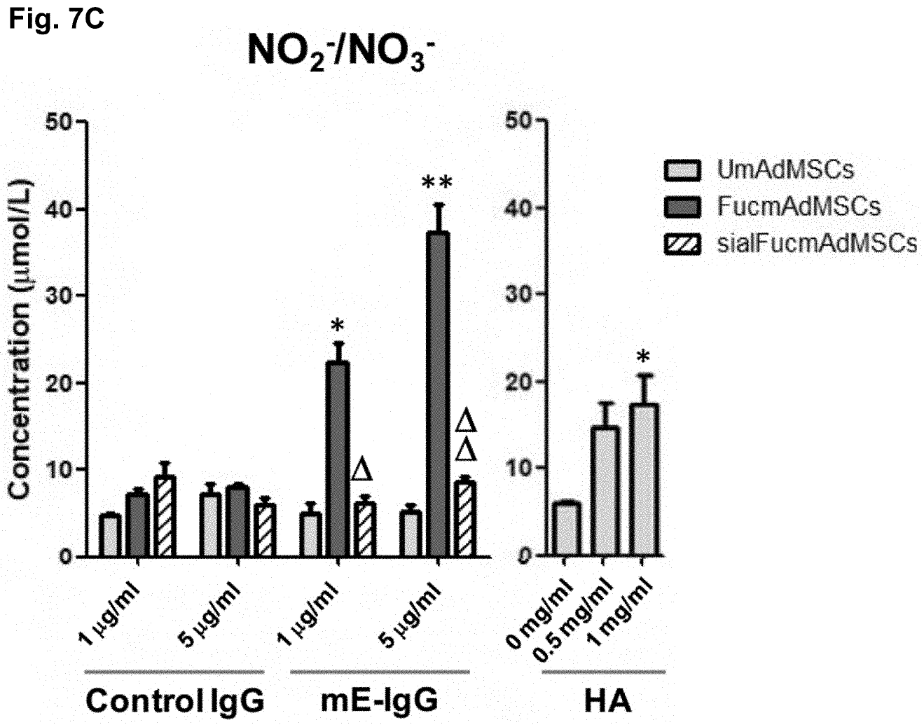

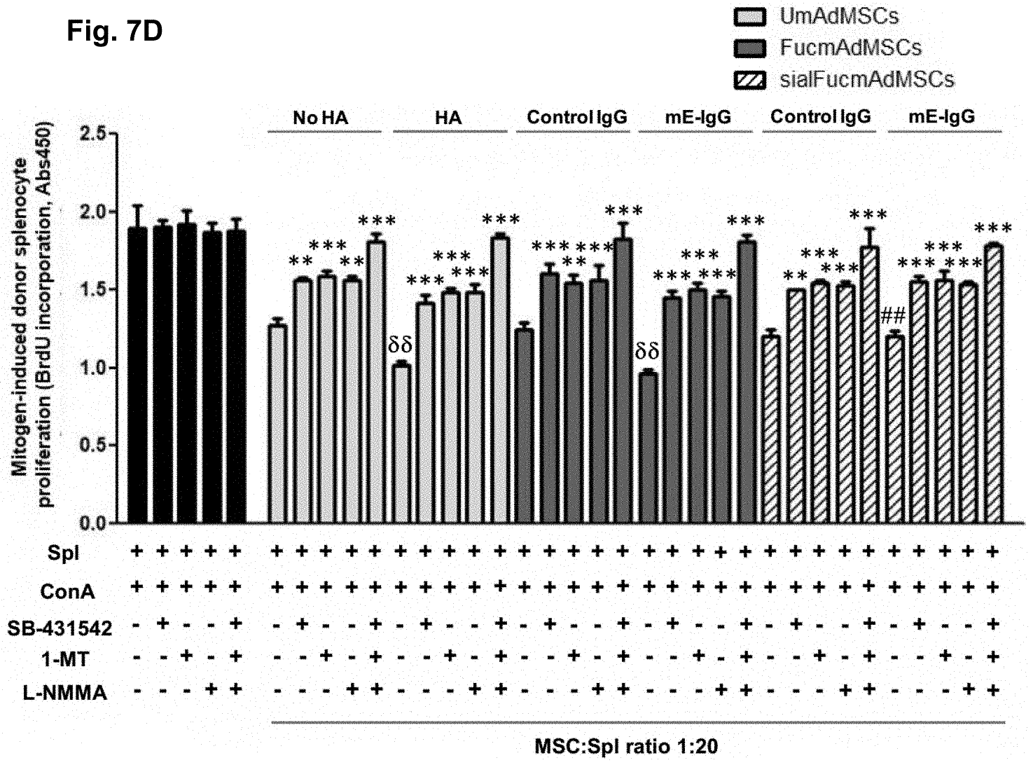

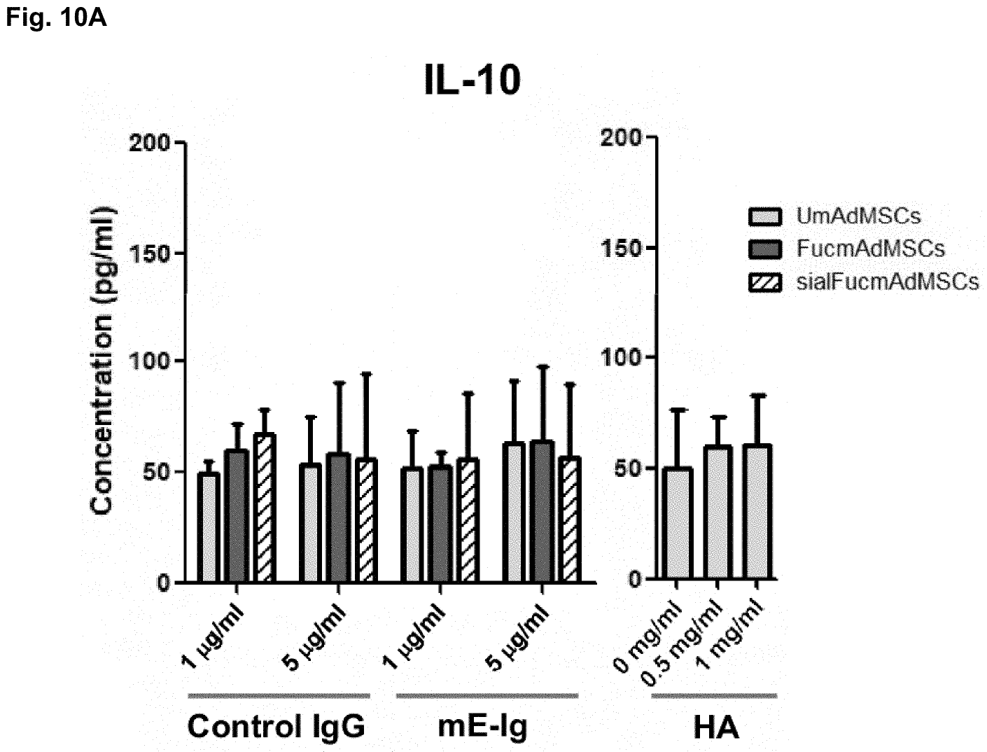

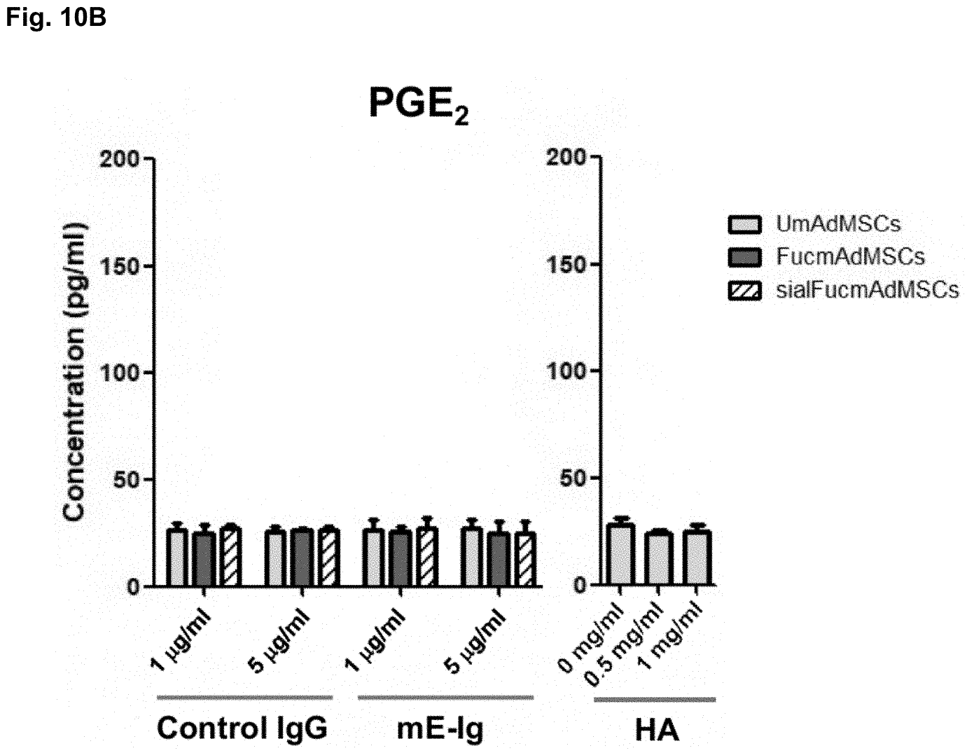

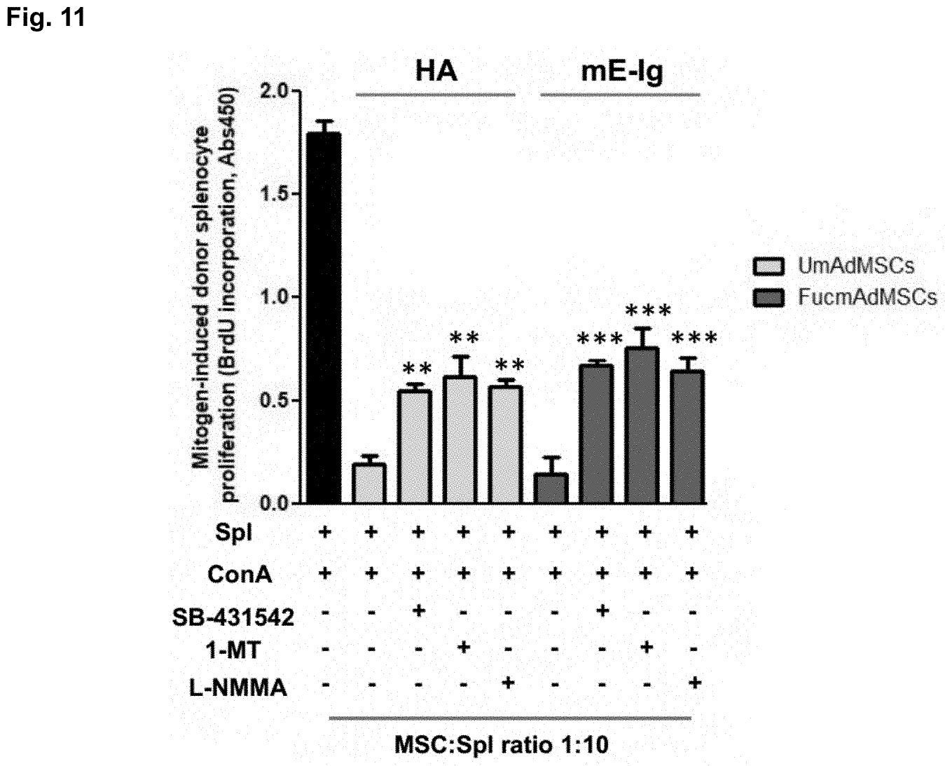

To ascertain whether MSC-lymphocyte cell-cell contacts are mandatory for the observed anti-proliferative effects of MSC CD44/HCELL ligation, supernatants were obtained of HCELL⁻ mAdMSCs and HCELL⁺ mAdMSCs after engagement with either HA or E-selectin and tested the effects of conditioned media on mitogen-induced splenocyte proliferation. As shown in FIG. 6G, media obtained from HA-ligated UmAdMSCs or HA-ligated sialFucmAdMSCs, and also from HA-ligated or E-selectin-ligated FucmAdMSCs, in each case profoundly inhibited mitogen-induced splenocyte proliferation (FIG. 6G, right) to an extent similar to that observed in continuous presence of MSCs (FIG. 6G, left). These findings indicate that secreted products of MSCs drive the observed dampening of splenocyte proliferation. Accordingly, to elucidate the relevant molecular effector(s), the supernatant levels were analyzed of TGFβ, IDO, nitrates/nitrites (e.g., nitric oxide (NO) metabolites), PGE₂, and IL-1β, each of which are reported to mediate immunosuppression. As shown in FIGS. 7A-7C and FIGS. 10A-10B, engagement of HCELL via E-selectin and of CD44 via HA on mAdMSCs, in each case, profoundly boosts levels of TGFβ, IDO, and NO metabolites (FIGS. 7A-7C); however, levels of IL-10 and PGE₂ on murine AdMSCs are unaffected by CD44/HCELL ligation (FIGS. 10A-10B). To further analyze the contributions of TGFβ, IDO, and NO to the observed mAdMSC anti-proliferative effect, in vitro mMSC:splenocyte co-cultures were performed in presence of inhibitors of these molecules at mAdMSC:splenocyte ratio of 1:20 (FIG. 7D) and of 1:10 (FIG. 11), with or without pre-incubation with HA or E-selectin. Addition of SB-431542 (TGFβ inhibitor), 1-methyl-DL-tryptophan (IDO inhibitor), or N^{G}-monomethyl-L-arginine (iNOS inhibitor) in each case significantly rescued the proliferation of mitogen-stimulated splenocytes, and simultaneous use of all three inhibitors resulted in complete recovery of proliferation (FIG. 7D). Collectively, these results indicate that these soluble molecules play pivotal roles in the mAdMSC-mediated immunomodulatory effects on activated splenocytes.

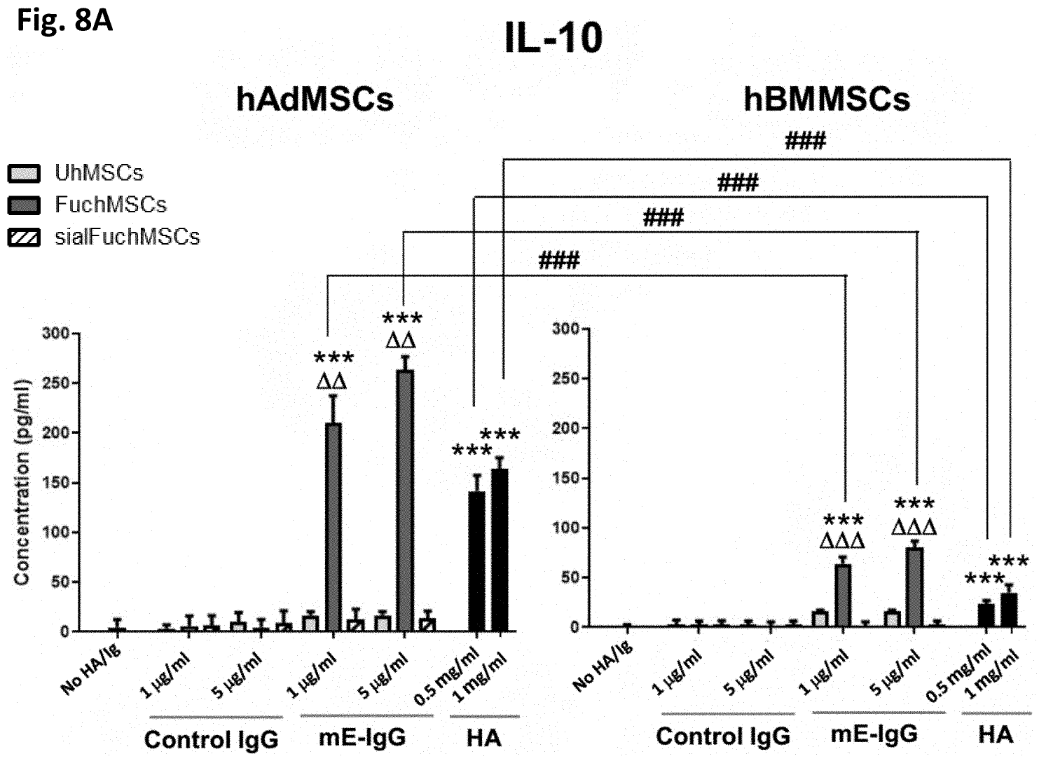

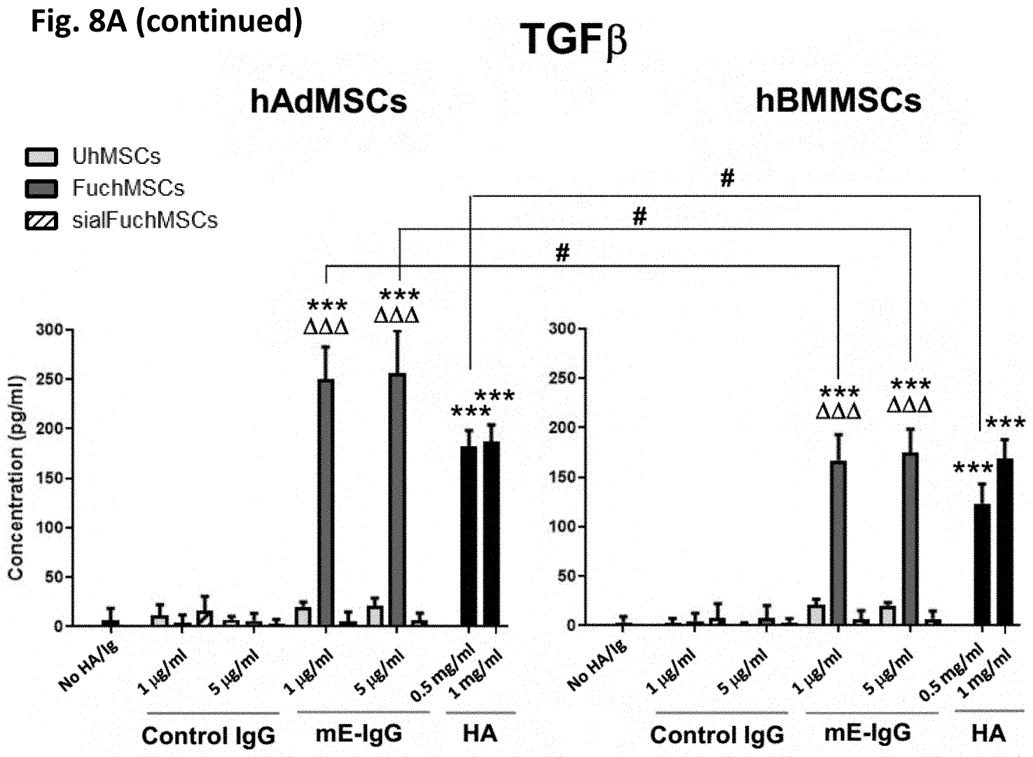

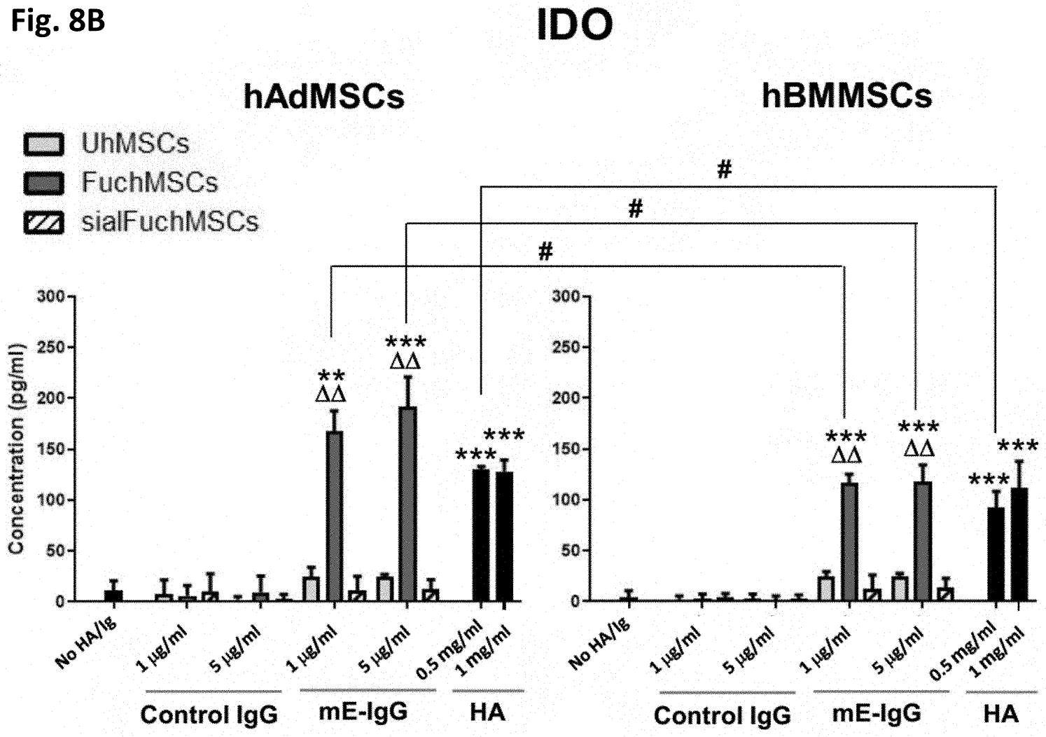

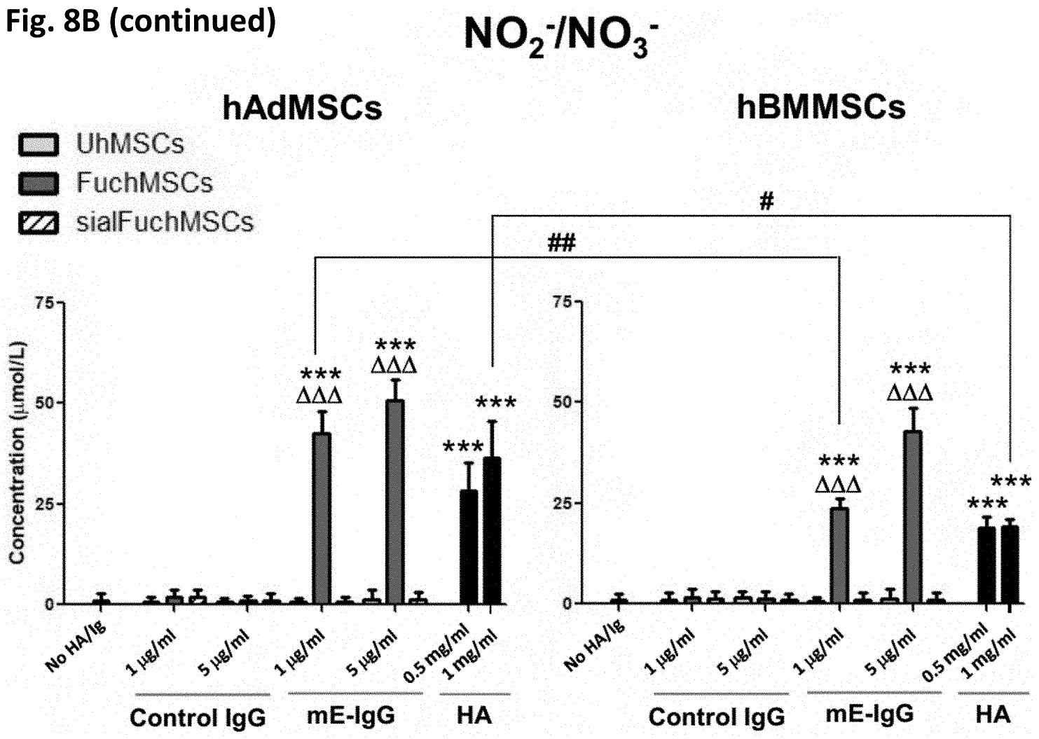

To analyze whether the observed immunomodulatory effect of CD44/HCELL engagement in murine MSCs is also characteristic of human MSCs (hMSCs), the levels of secreted anti-inflammatory molecules were measured after CD44/HCELL ligation of HCELL⁺ (i.e., exofucosylated) and HCELL⁻ hMSCs derived from both adipose tissue (hAdMSCs) and bone marrow (hBMMSCs) sources. Strikingly, just as observed in murine MSCs, both hAdMSCs and hBMMSCs each produced markedly higher levels of TGFβ, of IDO, and of NO metabolites after HCELL or CD44 ligation to E-selectin or HA, respectively (FIGS. 8A and 8B). However, in stark contrast to murine MSCs, CD44/HCELL ligation also profoundly boosted the production of the anti-inflammatory cytokine IL-10 by human MSCs from both marrow and adipose tissue sources (FIG. 8A). Notably, under stimulation by CD44/HCELL ligation with HA or E-selectin, respectively, production of all the immunomodulatory molecules tested was much higher in hAdMSCs than in hBMMSCs, especially for IL-10 (FIGS. 8A and 8B) (Table 2, showing comparisons of the levels of soluble immunomodulatory molecules in culture supernatants after E-selectin-mediated HCELL ligation (ratios of Fucosylated (“Fuc”)/unmodified (“U”) hMSCs) or HA-mediated CD44 ligation (ratios of HA-exposed (“w/HA”)/unexposed (“w/o HA”)) among culture-expanded adipose-derived human MSCs (hAdMSCs) and bone marrow-derived human MSCs (hBMMSCs).).

TABLE 2

| | Ratio | Ratio | Ratio | Ratio |
|---|---|---|---|---|
| Anti- | FuchAdMSCs/ | FuchBMMSCs/ | FuchAdMSCs/ | FuchBMMSCs/ |
| inflammatory | UhAdMSCs | UhBMMSCs | UhAdMSCs | UhBMMSCs |
| molecule | mE-IG (1 μg/ml) | mE-IG (1 μg/ml) | mE-IG (5 μg/ml) | mE-IG (5 μg/ml) |
| IL-10 | 12.74 ± 1.64 *** | 3.87 ± 0.41 | 15.96 ± 0.81 *** | 4.85 ± 0.41 |
| TGFβ | 12.16 ± 1.58 * | 8.11 ± 1.27 | 12.42 ± 2.10 * | 8.50 ± 1.13 |
| IDO | 6.75 ± 0.80 * | 4.71 ± 0.33 | 7.72 ± 1.18 * | 4.74 ± 0.68 |
| NO₂⁻/NO₃⁻ | 5.12 ± 0.52 ** | 3.31 ± 0.24 | 5.90 ± 0.51 | 5.14 ± 0.58 |
| | Ratio | Ratio | Ratio | Ratio |
| | UhAdMSCs | UhBMMSCs | UhAdMSCs | UhBMMSCs |
| | w/HA/ | w/HA/ | w/HA/ | w/HA/ |
| Anti- | UhAdMSCs | UhADMSCs | UhADMSCs | UhBMMSCs |
| inflammatory | w/o HA | w/o HA | w/o HA | w/o HA |
| molecule | HA (0.5 mg/ml) | HA (0.5 mg/ml) | HA (1 mg/ml) | HA (1 mg/ml) |
| IL-10 | 9.58 ± 0.96 *** | 2.42 ± 0.23 | 10.96 ± 0.67 *** | 3.13 ± 0.47 |
| TGFβ | 9.84 ± 0.79 * | 6.99 ± 0.79 | 10.10 ± 0.81 | 9.20 ± 0.93 |
| IDO | 6.23 ± 0.14 * | 4.73 ± 0.63 | 6.16 ± 0.46 | 5.50 ± 1.05 |
| NO₂⁻/NO₃⁻ | 3.73 ± 0.68 | 3.73 ± 0.68 | 4.52 ± 0.89 * | 2.86 ± 0.17 |

Indeed, in adipose-derived hAdMSCs, when analyzed as a ratio between IL-10 levels in hMSCs that engaged/did not engage either E-selectin or HA (i.e., ratio of FuchMSCs/UhMSCs for E-selectin exposure or of UhMSCs with HA exposure/UhMSCs without HA exposure), IL-10 production was heightened 10-fold by engagement of either HCELL (via E-selectin) or of CD44 (via HA); furthermore, following CD44/HCELL ligation, adipose-derived hMSCs consistently showed >3-fold higher production of IL-10 compared to that of bone marrow-derived hMSCs (Table 2).

### Example 3—Discussion

Despite many decades of studies on the pathobiology of immune diseases, there exists limited knowledge into how native components of the tissue microenvironment can ameliorate or prevent the attendant immunopathology in a site-specific manner. In “systemic” immune diseases, the presence of focal tissue-sparing within affected target organ(s) reflects the persistence of immunohomeostasis at those pertinent unaffected site(s), i.e., the preservation of native tissue integrity in the face of incipient inflammation indicates that sufficient amounts/density of immunomodulatory cellular elements are microenvironmentally present in situ to restrain the action(s) of inflammatory effectors. Every tissue of the body contains a reservoir of MSCs, but understanding of their immunobiology in vivo is obscure because there are no surface markers that uniquely identify these cells. As such, it is not possible to readily quantify MSC distribution within tissues, nor possible to monitor their response to inflammatory insults in situ. Moreover, MSCs do not emigrate from their parenchymal location(s) into blood stream, and they natively lack expression of “homing receptors” such as E-selectin ligands that guide their extravasation at distant inflammatory sites. Thus, to elucidate the immunoregulatory capacity of tissue-resident MSCs, it was sought to enforce colonization of culture-expanded E-selectin ligand-bearing MSCs at lesional sites given that their recruitment would be driven by upregulation of E-selectin expression within target endothelial beds at the onset/progression of immune-mediated tissue injury. Because donor splenocyte-enriched full MHC-mismatched transplants in mice reproducibly triggers aGVHD, a fulminant immunopathologic process, it was reasoned that enforced migration of MSCs to sites of aGVHD tissue injury would provide insights as to whether tissue-resident MSCs can promote and/or preserve immunohomeostasis.

The results disclosed herein provide direct evidence that enhancing the colonization of MSCs within inflamed tissues commensurately results in superior immunoregulation. Immunohistochemistry was performed to analyze the distribution of intravenously administered recipient-type HCELL⁺ and HCELL⁻ GFP⁺ mAdMSCs within a variety of tissues and consistently observed increased recruitment of HCELL⁺ mAdMSCs within aGVHD-affected sites as compared to that observed with infusion of HCELL⁻ mAdMSCs. MSC infiltrates were not detected in lymphoid tissues of mice receiving either type of AdMSCs, indicating that MSC infiltration within aGVHD-affected tissue itself, and not enhanced colonization of lymphoid tissues, elicits the observed immunomodulatory effect. The increased tissue residency of HCELL⁺ AdMSCs was associated with significant blunting of the severity of the evolving aGvHD, and strikingly increased animal survival compared to Untreated mice and those receiving HCELL⁻ AdMSCs. Though mice that received HCELL⁺ AdMSCs were not completely devoid of disease, a much lower lesional spectrum was observed: histological analysis revealed that administration of HCELL⁺ AdMSCs markedly attenuated aGvHD damage, prominently within the gastrointestinal tract and liver, compared to that observed in mice receiving HCELL⁻ AdMSCs and those that did not receive MSCs (“Untreated mice”). Moreover, the administration of HCELL⁺ AdMSCs yielded durable suppression of aGvHD, whereas administration of HCELL⁻ AdMSCs engendered only a partial and transient capacity to prevent/reverse aGvHD. Importantly, compared to early post-transplant administration of HCELL⁻ AdMSCs, administration of HCELL⁺ AdMSCs yielded increased MSC residency within the gut and liver parenchyma, and, in particular, dramatically increased MSC tropism to intestinal lamina propria.

The immunopathology of aGvHD is driven by immunoreactive donor effector T cells within target tissues, a process that takes place even during periods of lymphopenia and before engraftment (Sackstein, 2006). As expected, analysis of T cell infiltration in liver and gut from animals with ongoing aGvHD showed the presence of these effector cells early post-transplant. Compared to levels of T cell infiltrates in Untreated mice, administration of HCELL⁻ AdMSCs resulted in a modest decrease in T cell infiltrates in these organs. However, in mice receiving HCELL⁺ AdMSCs, conspicuously lower T cell infiltrates were observed at 10 and 20 days post-transplant, with a concomitant substantially reduced degree of tissue damage. The pattern of neutrophil infiltration mirrored that of T cells: following neutrophil engraftment (i.e., beyond day +10), markedly lower gut and liver infiltrates were observed in those mice receiving HCELL⁺ AdMSCs. Though not mediators of immunoreactivity per se, neutrophils contribute to tissue damage both directly (e.g., by release of ROS) and indirectly through the promotion of T cell activation (Schwab et al., 2014; Ziegler et al., 2001). Thus, the decreased neutrophil burden itself would lessen tissue injury, and abundant neutrophil infiltrates in aGvHD-target organs has been associated with increased early transplant-related mortality (Socie et al., 2004).

The infiltration of effector immune cells into tissues is associated with release of pro-inflammatory cytokines that have harmful effects in the affected organs and play a crucial role in the pathophysiology of immune-mediated diseases such as aGvHD (Hill et al., 1997; Reddy, 2003). It was observed here that mice treated with mAdMSC showed significantly decreased plasma levels of several pro-inflammatory cytokines compared to that of Untreated mice, yet this effect was not sustained in mice receiving HCELL⁻ AdMSCs (i.e., pro-inflammatory cytokine levels in UmAdMSC-treated mice dropped then gradually increased to that observed in Untreated animals by day +30) (FIG. 5). In contrast, animals treated with HCELL⁺ AdMSCs displayed a marked and prolonged decrease in plasma levels of pro-inflammatory mediators, and, moreover, these mice had significant increases in plasma levels of anti-inflammatory cytokines IL-10 and TGFβ (FIG. 5), each of which potently inhibit lymphocyte proliferation and promote tolerance (Fox et al., 1993; Taga and Tosato, 1992). In particular, increased levels of IL-10 could in itself profoundly dampen immunoreactivity, as this cytokine, initially recognized as the “cytokine synthesis inhibitory factor” (CSIF) (Fiorentino et al., 1989; Fiorentino et al., 1991; Macatonia et al., 1993; Saraiva et al., 2020; Vieira et al., 1991), has pleiotropic effects in not only decreasing the production of a variety of pro-inflammatory cytokines, but also in inhibiting T cell proliferation and decreasing the expression of MHC molecules and costimulatory molecules on antigen-presenting cells (Couper et al., 2008; de Waal Malefyt et al., 1991); in fact, through its potent role in immunosuppression, IL-10 is known to mediate tissue preservation in the face of exaggerated host immune responses to pathogens, thereby establishing host-pathogen immune equilibrium resulting in infectious latency (Belkaid et al., 2001). Moreover, apart from the observed increased plasma levels of anti-inflammatory cytokines, the ability of MSC administration to diminish pro-inflammatory cytokine levels would also serve to promote tissue preservation, limiting the systemic extension of immunopathology by decreasing immunoresponsiveness and decreasing the (cytokine-driven) induction of endothelial adhesion molecules that mediate recruitment of immune effector cells to inflammatory sites (Uccelli et al., 2008).

The results disclosed herein indicate that MSC-lymphocyte cell-cell contacts are not mandatory for MSC attenuation of mitogen-induced lymphocyte proliferation. Indeed, supernatants obtained from HCELL⁺ AdMSCs and from HCELL⁻ AdMSCs after pre-incubation with either E-selectin or HA, respectively, equally reduced mitogen-induced splenocyte proliferation to levels observed with continuous MSC contact (FIG. 6). These results indicate that secreted products of MSCs drive the observed anti-proliferative property. Analysis of the expression of TGFβ, IL-10, IDO, nitric oxide (NO) metabolites, and PGE₂ indicate that in vitro engagement of HCELL via E-selectin or HA, or of CD44 via HA, in each case profoundly boosts levels of TGFβ, IDO, and NO metabolites in both mouse (FIG. 7) and human MSCs (FIG. 8). Interestingly, ligation of HCELL or of CD44 in murine MSCs did not directly increase production of IL-10 (FIGS. 10A-10B). These data suggest that the observed increased plasma levels of IL-10 in mice receiving MSCs may be secondary to well-recognized indirect effects of MSCs in supporting/upregulating IL-10 production among other cell types that express this cytokine (e.g., monocytes, macrophages, dendritic cells, B cells and subsets of T cells (Couper et al., 2008); this function of MSCs has been reported to be potent enough in itself to drive immunomodulation in a variety of contexts (Aggarwal and Pittenger, 2005; Batten et al., 2006; Najar et al., 2015). In contrast, ligation of HCELL or of CD44 among human MSCs directly and strikingly boosts production of IL-10 (as well as TGFβ, IDO, and nitrates/nitrites) (FIG. 8). Importantly, following HCELL or CD44 engagement, adipose-derived human MSCs have much higher production of these agents than do marrow-derived human MSCs, and, conspicuously, IL-10 production by adipose-derived human MSCs is most profoundly induced (FIG. 8 and Table 2). Direct production of IL-10 by human MSCs (or by mouse MSCs) has not been reported previously, and the observed marked increased production of anti-inflammatory cytokines, especially IL-10, by human adipose-derived MSCs compared to human marrow-derived MSCs could alone account for the reported better immunomodulatory activity of these cells compared to that of marrow-derived human MSCs (Li et al., 2015; Valencia et al., 2016). Interestingly, the observed robust production of IL-10 by human adipose-derived MSCs may also reflect non-immunologic functions of this cytokine within adipose tissue as emerging data indicate that IL-10 could promote “metabolic syndrome” by its effects on limiting energy utilization and thermogenesis by adipocytes (Rajbhandari et al., 2018).

The heightened production of TGFβ, IDO and NO following engagement of FucmAdMSCs with E-selectin in vitro indicates that, following engagement with E-selectin displayed on vascular beds at inflammatory sites in vivo, HCELL⁺ mAdMSCs are primed to exert immunomodulatory effects within the inflammatory milieu via increased release of anti-inflammatory molecules (Groh et al., 2005; Lim et al., 2016; Meisel et al., 2004; Sato et al., 2007). This mechanism, together with the observed higher MSC tissue density in situ, underlies the observed improved clinical outcome of mice with fulminant immunoreactivity. Moreover, independent of E-selectin/HCELL interactions, engagement of MSC CD44 with its cognate ligand, HA, similarly boosts TGFβ, IDO, and NO production. Thus, once extravasated and localized within tissue parenchyma, MSC immunomodulatory properties would be unleashed through interaction of CD44 with HA, an integral component of the extracellular matrix whose expression is itself upregulated at sites of immunoreactivity/inflammation (Jiang et al., 2011; Petrey and de la Motte, 2014). Thus, the results herein offer novel mechanistic perspectives on how molecules within the inflammatory milieu can boost MSC capabilities as effectors of immunohomeostasis. Moreover, the data suggest that engagement of MSC CD44/HCELL with ligands such as HA/E-selectin prior to administration of the MSCs could be exploited to potentiate their in vivo role in dampening immunopathology.

Current therapies for immune-mediated diseases are primarily pharmacologic. New therapeutic approaches are urgently needed to improve patient outcomes as pharmacologic agents produce broad-spectrum immunosuppression and each are associated with significant adverse effects. Ideally, therapeutic immunomodulation should be concentrated solely at the site(s) of immunopathology, thereby establishing anatomically focal immunohomeostasis and preserving systemic immunoprotection. The findings disclosed herein indicate that MSC organ/tissue colonization engenders refractoriness to the pathologic consequences of immune-mediated inflammatory processes in situ. Accordingly, inflammation-induced expression of E-selectin within endothelial beds at affected sites could be leveraged for clinical benefit to achieve efficient tissue residency of systemically administered E-selectin ligand-bearing immunomodulatory MSCs at the desired anatomic location(s). Thus, rather than antagonizing its bioactivity (e.g., by use of anti-E-selectin mAb or mimetics of sLe^{X}) or its expression (e.g., by use of biologics blocking TNF or IL-1 action) (Lowe and Ward, 1997), pathophysiologic endothelial E-selectin display could serve as a gateway in ushering forth a new era of immunoregulatory cell-based therapies for inflammatory disorders.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes can be made by those skilled in the art without departing from the spirit of this disclosure. Those skilled in the art will appreciate that the inventive principles can be practiced in other than the described embodiments, which are presented for purposes of illustration and not limitation.

### Various Embodiments

Embodiment 1: A pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells.
Embodiment 2: A pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells.
Embodiment 3: A pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells.
Embodiment 4: A pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells.
Embodiment 5: The pharmaceutical composition according to any preceding Embodiment wherein the population of cells are mesenchymal stem cells (MSCs), hematopoietic stem cells, tissue stem/progenitor cells (for example, a neural stem cell, myocyte stem cell or pulmonary stem cell), stromal vascular fraction cells, umbilical cord-derived stem cells, or embryonic stem cells, induced pluripotent stem cells, differentiated progenitors derived from embryonic stem cells or from induced pluripotent stem cells, differentiated progenitors derived from adult stem cells, primary cells isolated from any tissue (e.g., blood, bone marrow, brain, liver, lung, gut, stomach, fat, muscle, testes, uterus, ovary, skin, spleen, eye, endocrine organ and bone), a culture-expanded progenitor cell population, a culture-expanded stem cell population, or a culture-expanded primary cell population.
Embodiment 6: The pharmaceutical composition according to any preceding Embodiment, wherein the population of cells are mesenchymal stem cells.
Embodiment 7: The pharmaceutical composition according to any preceding Embodiment, wherein the population of cells are culture-expanded mesenchymal stem cells.
Embodiment 8: The pharmaceutical composition according to any preceding Embodiment, wherein the population of cells are culture-expanded mammalian adipose-derived mesenchymal stem cells (AdMSCs).
Embodiment 9: The pharmaceutical composition according to any preceding Embodiment, wherein the population of cells are culture-expanded human adipose-derived mesenchymal stem cells (hAdMSCs).
Embodiment 10: The pharmaceutical composition according to Embodiment 9, wherein the culture-expanded hAdMSCs are modified ex vivo via treatment with HA.
Embodiment 11: The pharmaceutical composition according to any preceding Embodiment, wherein the at least one additional anti-inflammatory molecule is selected from TGF-β, IDO, nitric oxide (NO) metabolites, PGE₂ and combinations thereof.
Embodiment 12: The pharmaceutical composition according to any preceding Embodiment, wherein the IL-10 production is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of the cells.
Embodiment 13: The pharmaceutical composition according to any preceding Embodiment, wherein the IL-10 production is elevated at least 3-fold relative to a native population of the cells.
Embodiment 14: The pharmaceutical composition according to any preceding Embodiment, wherein the IL-10 production is elevated at least 10-fold relative to a native population of the cells.
Embodiment 15: The pharmaceutical composition according to any preceding Embodiment useful for decreasing plasma levels of at least one pro-inflammatory molecule in a subject when administered to the subject.
Embodiment 16: The pharmaceutical composition according to Embodiment 15, wherein the at least one pro-inflammatory molecule comprises a group selected from IFNγ, TNFα, IL-1α, IL-1β, IL-6, IL-12, IL-17 and combinations thereof.
Embodiment 17: The pharmaceutical composition according to any preceding Embodiment, wherein the E-Selectin or L-selectin is an E-Selectin-immunoglobulin or L-selectin-immunoglobulin chimera (E-Ig chimera or L-Ig chimera).
Embodiment 18: The pharmaceutical composition according to any preceding Embodiment useful for the treatment of a disease associated with one or more of neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, systemic inflammatory response syndrome, acute respiratory disease syndrome secondary to bacteria, fungi or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.).
Embodiment 19: The pharmaceutical composition according to any preceding Embodiment useful for the treatment of a disease associated with a cytokine storm.
Embodiment 20: The pharmaceutical composition according to any preceding Embodiment useful for engendering immunohomeostasis in a subject.
Embodiment 21: The pharmaceutical composition according to any preceding Embodiment useful for the treatment of graft versus host (GvH) disease.
Embodiment 22: The pharmaceutical composition according to any preceding Embodiment useful for the treatment of COVID-19 infection or sequelae of COVID-19 infection (e.g., Kawasaki disease).
Embodiment 23: The pharmaceutical composition according to any preceding Embodiment, wherein the subject is a human.
Embodiment 24: A population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs.
Embodiment 25: A population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs.
Embodiment 26: A population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.
Embodiment 27: A population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs.
Embodiment 28: A population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs.
Embodiment 29: A population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.
Embodiment 30: The population of any one of Embodiments 24-29 which is culture-expanded.
Embodiment 31: The population of any one of Embodiments 24-26 in which the HA-primed cells are exofucosylated to enforce hematopoietic cell E-Selectin/L-Selectin Ligand (HCELL) prior to administration to a subject.
Embodiment 32: The population of any one of Embodiments 27-29 in which the E-Selectin or L-selectin-primed cells are exofucosylated a second time in vitro to enforce hematopoietic cell E-Selectin/L-Selectin Ligand (HCELL) prior to administration to a subject.
Embodiment 33: The population of any one of Embodiments 27-30 and 32, wherein the E-Selectin or L-selectin is an E-Selectin-immunoglobulin or L-selectin-immunoglobulin chimera (E-Ig chimera or L-Ig chimera).
Embodiment 34: The population of any one of Embodiments 24-33, wherein the IL-10 production is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of the cells.
Embodiment 35: The population according to any one of Embodiments 24-33, wherein the IL-10 production is elevated at least 3-fold relative to a native population of the cells.
Embodiment 36: The population of any one of Embodiments 24-33, wherein the IL-10 production is elevated at least 10-fold relative to a native population of the cells.
Embodiment 37: The population according to any one of Embodiments 24-36 useful for decreasing plasma levels of at least one pro-inflammatory molecule when administered to a subject.
Embodiment 38: The population according to Embodiment 37, wherein the at least one pro-inflammatory molecule comprises a group selected from IFNγ, TNFα, IL-1α, IL-1β, IL-6, IL-12, IL-17 and combinations thereof.
Embodiment 39: The population according to any one of Embodiments 24-38, wherein the at least one additional anti-inflammatory molecule is selected from TGF-β, IDO, nitric oxide (NO) metabolites, PGE₂ and combinations thereof.
Embodiment 40: The population according to any one of Embodiments 24-39 useful for the treatment of a disease associated with one or more of neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, systemic inflammatory response syndrome, acute respiratory disease syndrome secondary to bacteria, fungi or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.).
Embodiment 40A: The population according to any one of Embodiments 24-39 useful for the treatment of multi-system inflammatory syndrome.
Embodiment 41: The population according to any one of Embodiments 24-39 useful for the treatment of a disease associated with a cytokine storm.
Embodiment 42: The population according to any one of Embodiments 24-39 useful for engendering immunohomeostasis in a subject.
Embodiment 43: The population according to any one of Embodiments 24-39 useful for the treatment of graft versus host (GvH) disease.
Embodiment 44: The population according to any one of Embodiments 24-39 useful for the treatment of COVID-19 infection.
Embodiment 45: The population according to any one of Embodiments 24-44, wherein the subject is a human.
Embodiment 46: A unit dose of the population according to any one of Embodiments 24-45 comprising an effective amount of the primed cells.
Embodiment 47: The unit dose according to Embodiment 46, which effective amount is selected from at least about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg.
Embodiment 48: The unit dose according to Embodiment 46, which effective amount is less than about one million primed cells/kg.
Embodiment 49: The unit dose according to Embodiment 46, which effective amount is between about 50,000 to about 950,000 primed cells/kg.
Embodiment 50: A method of treating a disease or disorder associated with elevated levels of at least one pro-inflammatory molecule in a subject comprising administering to the subject:
   (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (iv) a pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs;
   (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs;
   (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule;
   (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs;
   (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or
   (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.
Embodiment 51: The method according to Embodiment 50 or composition for use according to Embodiment 138, wherein the disease or disorder associated with elevated levels of at least one pro-inflammatory molecule is selected from neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, systemic inflammatory response syndrome, acute respiratory disease syndrome secondary to bacteria, fung or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.).
Embodiment 51A: The method according to Embodiment 50 or composition for use according to Embodiment 138, wherein the disease or disorder associated with elevated levels of at least one pro-inflammatory molecule is multi-system inflammatory syndrome.
Embodiment 52: The method according to Embodiment 50 or composition for use according to Embodiment 138, wherein the disease or disorder associated with elevated levels of at least one pro-inflammatory molecule is a cytokine storm.
Embodiment 53: The method according to Embodiment 50 or composition for use according to Embodiment 138, wherein the disease or disorder associated with elevated levels of at least one pro-inflammatory molecule is graft versus host (GvH) disease.
Embodiment 54: The method according to Embodiment 50 or composition for use according to Embodiment 138, wherein the disease or disorder is associated with elevated levels of at least one pro-inflammatory molecule (e.g., IL-6).
Embodiment 55: The method according to Embodiment 50 or composition for use according to Embodiment 138, wherein the E-selectin or L-selectin-primed cells are further exofucosylated prior to administration to the subject.
Embodiment 56: The method according to Embodiment 50 or composition for use according to Embodiment 138, wherein the HA-primed cells are exofucosylated prior to administration to the subject.
Embodiment 57: A method of modulating the effects of a cytokine storm in a subject, the method comprising administering to the subject before, during or after onset of the cytokine storm:
   (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (iv) a pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs;
   (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs;
   (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule;
   (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs;
   (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or
   (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.
Embodiment 58: The method according to any one of Embodiments 50-57 or composition for use according to Embodiment 139, wherein the IL-10 production is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of cells.
Embodiment 58A: The method according to any one of Embodiments 50-57 or composition for use according to Embodiment 139, wherein the IL-10 production or the production of the at least one additional anti-inflammatory molecule is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of cells.
Embodiment 59: The method according to any one of Embodiments 50-57 or composition for use according to Embodiment 139, wherein the IL-10 production is elevated at least 3-fold relative to a native population of cells.
Embodiment 60: The method according to any one of Embodiments 50-57 or composition for use according to Embodiment 139, wherein the IL-10 production is elevated at least 10-fold relative to a native population of cells.
Embodiment 61: The method according to any one of Embodiments 50-57 or composition for use according to Embodiment 139, wherein the at least one pro-inflammatory molecule comprises a group selected from IFNγ, TNFα, IL-1α, IL-1β, IL-6, IL-12, IL-17 and combinations thereof.
Embodiment 62: The method according to any one of Embodiments 50-57 or composition for use according to Embodiment 139, wherein the at least one pro-inflammatory molecule is the cytokine IL-6.
Embodiment 63: The method according to any one of Embodiments 50-62 or composition for use according to Embodiment 139, wherein the at least one additional anti-inflammatory molecule is selected from TGF-β, IDO, nitric oxide (NO) metabolites, PGE₂ and combinations thereof.
Embodiment 64: The method according to any one of Embodiments 50-63 or composition for use according to Embodiment 139, wherein the increase in IL-10 production and decrease in plasma levels of at least one pro-inflammatory molecule is observed for a prolonged period of time.
Embodiment 65: The method according to Embodiment 64 or composition for use according to Embodiment 139, wherein the prolonged period of time is for at least 5 days, 10 days, at least 20 days or at least 30 days.
Embodiment 66: The method according to any one of Embodiments 50-65 or composition for use according to Embodiment 139, wherein the pharmaceutical composition or population of cells is administered to the subject topically, intravascularly, by direct injection, or as an aerosol.
Embodiment 67: The method according to any one of Embodiments 50-66 or composition for use according to Embodiment 139 further comprising administering the pharmaceutical composition or population of cells as an adjuvant to a primary immunotherapy.
Embodiment 68: The method according to any one of Embodiments 50-67 or composition for use according to Embodiment 139, wherein the administration step comprises delivering to the subject about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg.
Embodiment 69: The method according to any one of Embodiments 50-67 or composition for use according to Embodiment 139, wherein the administration step comprises delivering to the subject less than about one million primed cells/kg.
Embodiment 70: The method according to any one of Embodiments 50-67 or composition for use according to Embodiment 139, wherein the administration step comprises delivering to the subject between about 50,000 to about 950,000 primed cells/kg.
Embodiment 71: The method according to any one of Embodiments 50-70 or composition for use according to Embodiment 139, wherein the subject is a human.
Embodiment 72: The method according to Embodiment 57-71 or composition for use according to Embodiment 139, wherein the E-selectin- or L-selectin-primed cells are further exofucosylated prior to administration to the subject.
Embodiment 73: The method according to Embodiment 57-71 or composition for use according to Embodiment 139, wherein the HA-primed cells are exofucosylated prior to administration to a subject.
Embodiment 74: A low dose pharmaceutical composition in unit dosage form for intravascular (e.g., intravenous), direct injection, topical or aerosol delivery to a subject comprising:
   (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻CD44⁺/PSGL⁻ cells;
   (iv) a pharmaceutical composition comprising conditioned media obtained from a population of CD34⁻/CD44⁺/PSGL⁻CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻CD44⁺/PSGL⁻ cells;
   (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs;
   (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs;
   (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule;
   (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs;
   (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or
   (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule.
Embodiment 75: The low dose pharmaceutical composition according to Embodiment 74, which comprises about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg.
Embodiment 76: The low dose pharmaceutical composition according to Embodiment 74, which comprises less than about one million primed cells.
Embodiment 77: The low dose pharmaceutical composition according to Embodiment 74, which comprises between about 50,000 to about 950,000 primed cells/kg.
Embodiment 78: The low dose pharmaceutical composition according to any one of Embodiments 74-77, wherein the E-selectin or L-selectin-primed cells are further exofucosylated prior to administration to a subject.
Embodiment 79: The low dose pharmaceutical composition according to any one of Embodiments 74-77, wherein the HA-primed cells are exofucosylated prior to administration to a subject.
Embodiment 80: A method of producing a pharmaceutical composition for treating a disease or disorder associated with elevated levels of at least one pro-inflammatory molecule in a subject comprising: ex vivo exofucosylating a stem cell and culturing the stem cell under conditions sufficient to enforce hematopoietic cell E-Selectin/L-Selectin Ligand (HCELL) expression on a surface of the stem cell at levels above what is natively present on the stem cell and treating the HCELL⁺ stem cell with E-selectin or L-selectin to prime the HCELL⁺ stem cells to (a) produce elevated levels of interleukin-10 (IL-10) and at least one additional anti-inflammatory molecule relative to a native population of the stem cells and/or to (b) induce a decrease in the plasma levels of at least one pro-inflammatory molecule when administered to a subject.
Embodiment 81: A method of producing a pharmaceutical composition for treating a disease or disorder associated with elevated levels of at least one pro-inflammatory molecule in a subject comprising: ex vivo ligating CD44 on a surface of a stem cell with hyaluronic acid (HA) and culturing the stem cell under conditions sufficient to prime the stem cell to (a) produce elevated levels of interleukin-10 (IL-10) and at least one additional anti-inflammatory molecule relative to a native population of the stem cells and/or to (b) induce a decrease in the plasma levels of at least one pro-inflammatory molecule when administered to a subject.
Embodiment 82: The method according to Embodiment 80 further comprising culture expanding the stem cell prior to exofucosylation.
Embodiment 83: The method according to Embodiment 81 further comprising culture expanding the stem cell prior to ligation of CD44 with HA.
Embodiment 84: The method according to any one of Embodiments 80-83 further comprising preparing a unit dosage form comprising about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg.
Embodiment 85: The method according to any one of Embodiments 80-83 further comprising preparing a unit dosage form comprising less than about one million primed cells.
Embodiment 86: The method according to any one of Embodiments 80-83 further comprising preparing a unit dosage form comprising between about 50,000 to about 950,000 primed cells/kg.
Embodiment 87: The method according to Embodiment 80 further comprising exofucosylating the E-selectin or L-selectin-primed cells prior to administration to a subject.
Embodiment 88: The method according to Embodiment 81, wherein the culture conditions comprise incubating the HA with the cells for up to about 72 hours.
Embodiment 89: The method according to Embodiment 81, wherein the culture conditions comprise incubating the HA with the cells for at least about 24 hours.
Embodiment 90: The method according to Embodiment 81, wherein the culture conditions comprise incubating the HA with the cells for between about 24-72 hours.
Embodiment 91: The method according to Embodiment 81 further comprising exofucosylating the HA-primed cells prior to administration to a subject.
Embodiment 92: The method according to any one of Embodiments 80-91, wherein the stem cells are harvested from the subject prior to the ex vivo modification.
Embodiment 93: The method according to any one of Embodiments 80-91, wherein the stem cells are harvested from a compatible donor prior to the ex vivo modification.
Embodiment 94: The method according to any one of Embodiments 80, 87 and 91, wherein the exofucosylation is carried out with a glycosyltransferase together with donor nucleotide sugar.
Embodiment 95: The method according to Embodiment 94, wherein the glycosyltransferase is an alpha 1,3-fucosyltransferase.
Embodiment 96: The method according to Embodiment 95, wherein the alpha 1,3-fucosyltransferase is alpha 1,3-fucosyltransferase FTIII, FTIV, FTV, FTVI, FTVII, and combinations thereof.
Embodiment 97: The method according to Embodiment 96, wherein the alpha 1,3-fucosyltransferase is human FTVII.
Embodiment 98: The method according to any one of Embodiments 80-97, wherein the stem cell is selected from the group consisting of embryonic stem cells, adult stem cells, hematopoietic stem cells and induced pluripotent stem cells (iPSCs).
Embodiment 99: The method according to any one of Embodiments 80-97, wherein the stem cell is a MSC.
Embodiment 100: The method according to any one of Embodiments 80-97, wherein the stem cell is an AdMSC.
Embodiment 101: The method according to any one of Embodiments 80-97, wherein the stem cell is a hAdMSC.
Embodiment 102: The method according to any one of Embodiments 80-101 further comprising harvesting conditioned media from the modified cells.
Embodiment 103: A pharmaceutical composition produced by the method of any one of Embodiments 80-102.
Embodiment 104: A method of treating a disease or disorder associated with elevated levels of at least one pro-inflammatory molecule in a subject comprising:
   (i) preparing a pharmaceutical composition according to the method of any one of Embodiments 80-102; and
   (ii) administering the pharmaceutical composition from step (i) to the subject.
Embodiment 105: The method according to Embodiment 104, wherein the administering step comprises intravascular, direct injection, topical or aerosol delivery to the subject.
Embodiment 106: The method according to Embodiment 104 or composition for use according to Embodiment 140, wherein the pharmaceutical composition is administered to the subject in a low dose unit dosage form.
Embodiment 107: The method according to Embodiment 106, wherein the low dose comprises about 50,000 primed cells/kg, at least about 200,000 primed cells/kg, at least about 400,000 primed cells/kg, at least about 500,000 primed cells/kg, at least about 600,000 primed cells/kg, at least about 700,000 primed cells/kg, at least about 800,000 primed cells/kg, or at least about 900,000 primed cells/kg.
Embodiment 108: The method according to Embodiment 106, wherein the low dose comprises less than about one million primed cells.
Embodiment 109: The method according to Embodiment 106, wherein the low dose comprises between about 50,000 to about 950,000 primed cells/kg.
Embodiment 110: A method of selecting a population of CD44⁺ cells that are effective for treatment of inflammatory disorders comprising the steps of:
   (i) modifying ex vivo the population of CD44⁺ cells via exofucosylation to enforce hematopoietic cell E-Selectin/L-Selectin Ligand (HCELL) expression and treating the ex vivo modified cells with E-selectin or L-selectin; or modifying ex vivo the population of CD44⁺ cells via treatment with hyaluronic acid (HA);
   (ii) detecting in the modified population of CD44⁺ cells of step (1) production of an anti-inflammatory or immunomodulatory molecule; and
   (iii) selecting the modified cells that produce elevated levels of the anti-inflammatory or immunomodulatory molecule relative to a native population of CD44⁺ cells for use in the treatment of inflammatory disorders.
Embodiment 111: The method, composition or population according to any preceding Embodiment comprising enhancing CD44-HA, HCELL-HA, HCELL-E-Selectin, or HCELL⁻ L-selectin binding with an agent.
Embodiment 112: The method according to Embodiment 111, wherein the agent is an antibody to CD44 or antigen binding fragment thereof that cross-links CD44 or that functions to upregulate the ability of CD44⁺ cells to bind HA or that functions to enhance HCELL binding to E-Selectin or L-selectin.
Embodiment 113: A population of hAdMSCs, in isolated form, that express hematopoietic cell E-Selectin/L-Selectin Ligand (HCELL) at a level that exceeds the level of HCELL expression by native hAdMSCs as assessed by Western blot using monoclonal antibody HECA-452 and express interleukin-10 (IL-10) and at least one additional anti-inflammatory molecule at a level that exceeds the level of expression of each such molecule by native hAdMSCs as assessed using culture supernatant by ELISA or a modified Griess reagent in the case of nitric oxide (NO) metabolites.
Embodiment 114: A pharmaceutical composition for administration to a subject comprising a population of hAdMSCs that express (i) hematopoietic cell E-Selectin/L-Selectin Ligand (HCELL) at a level that exceeds the level of HCELL expression by native hAdMSCs as assessed by Western blot using monoclonal antibody HECA-452 and (ii) interleukin-10 (IL-10) that exceeds the level of expression of IL-10 by native hAdMSCs as assessed using culture supernatant by ELISA.
Embodiment 115: The pharmaceutical composition according to Embodiment 114, wherein the population of hAdMSCs express at least one additional anti-inflammatory molecule that exceeds the level of production of such anti-inflammatory molecule relative to native hAdMSCs as assessed using culture supernatant by ELISA or a modified Griess reagent in the case of nitric oxide (NO) metabolites.
Embodiment 116: The pharmaceutical composition according to Embodiment 114 in dosage unit form comprising the population of hAdMSCs and a pharmaceutically acceptable excipient, wherein the population of hAdMSCs contained within the dosage unit does not exceed one million hAdMSC cells.
Embodiment 117: A method of treating a disease or disorder associated with elevated levels of at least one pro-inflammatory molecule in a subject comprising administering to the subject the population of hAdMSCs according to Embodiment 113 or the pharmaceutical composition according to any one of Embodiments 114-116.
Embodiment 118: The method according to Embodiment 117, wherein the administration step is selected from intravascular, direct injection, topical or aerosol delivery to the subject.
Embodiment 119: Use of the population of hAdMSCs according to Embodiment 113 or the pharmaceutical composition according to any one of Embodiments 114-116 for use in the treatment of a disease or disorder associated with elevated levels of at least one pro-inflammatory molecule.
Embodiment 120: A pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells.
Embodiment 121: A pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells.
Embodiment 122: A population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of CD44⁺ cells.
Embodiment 123: A method of treating a disease or disorder associated with elevated levels of at least one pro-inflammatory molecule in a subject comprising administering to the subject:
   (i) a pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells;
   (ii) a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or
   (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs.
Embodiment 124: A method of modulating the effects of a cytokine storm in a subject, the method comprising administering to the subject before, during or after onset of the cytokine storm:
   (i) a pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells;
   (ii) a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or
   (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs.
Embodiment 125: A low dose pharmaceutical composition in unit dosage form for intravascular, direct injection, topical or aerosol delivery to a subject comprising:
   (i) a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells;
   (ii) a conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or
   (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs.
Embodiment 126: The pharmaceutical composition of any one of Embodiments 120, 121 and 125 or the population of Embodiment 122 or the method of any one of Embodiments 123 or 124 or the composition for use of Embodiment 142 or 143, wherein the CD44 ligand is a naturally occurring ligand.
Embodiment 127: The pharmaceutical composition of any one of Embodiments 120, 121 and 125 or the population of Embodiment 122 or the method of any one of Embodiments 123 or 124 or the composition for use of Embodiment 142 or 143, wherein the CD44 ligand is an artificial ligand.
Embodiment 128: The pharmaceutical composition of any one of Embodiments 120, 121 and 125 or the population of Embodiment 122 or the method of any one of Embodiments 123 or 124 or the composition for use of Embodiment 142 or 143, wherein a naturally occurring glycan modification of CD44 is altered ex vivo to permit and/or promote binding of a ligand to CD44.
Embodiment 129: The pharmaceutical composition of any one of Embodiments 120, 121 and 125 or the population of Embodiment 122 or the method of any one of Embodiments 123 or 124 or the composition for use of Embodiment 142 or 143, wherein the CD44⁺ cells have been modified ex vivo via sialidase to remove terminal sialic acids on CD44 O-glycans or N-glycans and treated with HA.
Embodiment 130: The pharmaceutical composition of any one of Embodiments 120, 121 and 125 or the population of Embodiment 122 or the method of any one of Embodiments 123 or 124 or the composition for use of Embodiment 142 or 143, wherein a glycan decorating the CD44 is altered to promote binding of one or more selectins.
Embodiment 131: The pharmaceutical composition of any one of Embodiments 120, 121 and 125 or the population of Embodiment 122 or the method of any one of Embodiments 123 or 124 or the composition for use of Embodiment 142 or 143, wherein the CD44⁺ cells are treated ex vivo with one or more fucosyltransferases to enforce expression of HCELL and to promote binding to E-selectin or L-selectin.
Embodiment 132: The pharmaceutical composition of any one of Embodiments 120, 121 and 125 or the population of Embodiment 122 or the method of any one of Embodiments 123 or 124 or the composition for use of Embodiment 142 or 143, wherein a glycosyltransferase is used to install a chemically reactive group, orthogonal functional group, or molecular tag on the CD44, and ligand binding to the chemically reactive group, orthogonal functional group, or molecular tag is effective to promote production of the elevated levels of the one or more anti-inflammatory or immunomodulatory molecules.
Embodiment 133: The pharmaceutical composition of any one of Embodiments 120, 121 and 125 or the population of Embodiment 122 or the method of any one of Embodiments 123 or 124 or the composition for use of Embodiment 142 or 143, wherein the CD44 is ligated with an agent effective to promote the elevated levels of the one or more anti-inflammatory or immunomodulatory molecules.
Embodiment 134: The pharmaceutical composition of any one of Embodiments 120, 121 and 125 or the population of Embodiment 122 or the method of any one of Embodiments 123 or 124 or the composition for use of Embodiment 142 or 143, wherein the CD44⁺ cells have been modified ex vivo to express HCELL and treated with one or more of E-selectin, L-selectin, CSLEX-1 mAbs, and HECA452 mAbs.
Embodiment 135: The pharmaceutical composition of any one of Embodiments 120, 121 and 125 or the population of Embodiment 122 or the method of any one of Embodiments 123 or 124 or the composition for use of Embodiment 142 or 143, wherein the CD44⁺ cells are mesenchymal stem cells (MSCs), hematopoietic stem cells, tissue stem/progenitor cells, umbilical cord-derived stem cells, stromal vascular fraction, or embryonic stem cells, induced pluripotent stem cells, differentiated progenitors derived from embryonic stem cells or from induced pluripotent stem cells, differentiated progenitors derived from adult stem cells, primary cells isolated from blood or any tissue, a culture-expanded progenitor cell population, a culture-expanded stem cell population, or a culture-expanded primary cell population.
Embodiment 136: The pharmaceutical composition of any one of Embodiments 120, 121 and 125 or the population of Embodiment 122 or the method of any one of Embodiments 123 or 124 or the composition for use of Embodiment 142 or 143, wherein each one or more anti-inflammatory or immunomodulatory molecule is the same or different molecule.
Embodiment 137: The pharmaceutical composition of any one of Embodiments 120, 121 and 125 or the population of Embodiment 122 or the method of any one of Embodiments 123 or 124 or the composition for use of Embodiment 142 or 143, wherein the one or more anti-inflammatory or immunomodulatory molecule comprises IL-10.
Embodiment 137A: The pharmaceutical composition of any one of Embodiments 120, 121 and 125 or the population of Embodiment 122 or the method of any one of Embodiments 123 or 124 or the composition for use of Embodiment 142 or 143 wherein the level of the one or more anti-inflammatory or immunomodulatory molecules is increased by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of CD44⁺ cells
Embodiment 138: A composition comprising:
   (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (iv) a pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs;
   (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs;
   (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule;
   (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs;
   (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or
   (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule; for use in the treatment of a disease or disorder associated with elevated levels of at least one pro-inflammatory molecule in a subject.
Embodiment 139: A composition comprising:
   (i) a pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (ii) a pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells;
   (iii) a pharmaceutical composition comprising a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (iv) a pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells;
   (v) a population of mesenchymal stem cells (MSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of MSCs;
   (vi) a population of human adipose-derived MSCs (hAdMSCs), in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs;
   (vii) a population of hAdMSCs, in isolated form, that have been ex vivo treated with hyaluronic acid for a period of time sufficient to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule;
   (viii) a population of mesenchymal stem cells (MSCs), in isolated form, that have been ex vivo exofucosylated to enforce hematopoietic cell E-Selectin/L-Selectin Ligand (HCELL) expression and treated with E-selectin or L-selectin for a period of time sufficient to prime the cells to produce elevated levels of interleukin-10 (IL-10) and at least one additional anti-inflammatory molecule relative to a native population of MSCs;
   (ix) a population of human adipose-derived MSCs (hAdMSCs), in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs; or
   (x) a population of hAdMSCs, in isolated form, that have been ex vivo exofucosylated to enforce hematopoietic cell E-Selectin/L-Selectin Ligand (HCELL) expression and treated with E-selectin or L-selectin to prime the cells to produce elevated levels of interleukin-10 (IL-10) and at least one additional anti-inflammatory molecule relative to a native population of hAdMSCs and when administered to a subject induce a decrease in plasma levels of at least one pro-inflammatory molecule; for use in modulating the effects of a cytokine storm in a subject before, during or after onset of the cytokine storm.
Embodiment 140: A composition comprising a pharmaceutical composition prepared by the method of any one of Embodiments 80-102 for use in treating a disease or disorder associated with elevated levels of at least one pro-inflammatory molecule in a subject.
Embodiment 141: A composition comprising the population of hAdMSCs according to Embodiment 113 or the pharmaceutical composition according to any one of Embodiments 114-116 for use in the treatment of a disease or disorder associated with elevated levels of at least one pro-inflammatory molecule in a subject.
Embodiment 142: A composition comprising:
   (i) a pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells;
   (ii) a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or
   (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs; for use in the treatment of a disease or disorder associated with elevated levels of at least one pro-inflammatory molecule in a subject.
Embodiment 143: A composition comprising:
   (i) a pharmaceutical composition comprising a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells;
   (ii) a pharmaceutical composition comprising conditioned media obtained from a population of CD44⁺ cells that have been modified ex vivo via treatment with a CD44 ligand for a period of time sufficient to prime the cells to produce elevated levels of one or more anti-inflammatory or immunomodulatory molecules relative to a native population of CD44⁺ cells; or
   (iii) a population of mesenchymal stem cells (MSCs), in isolated form, that have been ex vivo treated with a CD44 ligand for a period of time sufficient to prime the cells to produce elevated levels of one or more anti-inflammatory or immunomodulatory molecules relative to a native population of MSCs; for use in modulating the effects of a cytokine storm in a subject before, during or after onset of the cytokine storm.
Embodiment 144: The method according to Embodiments 50, 104, or 120 wherein the pharmaceutical composition or population is effective to increase the local level of one or more anti-inflammatory molecules upon local administration to a lesional site in a subject by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to the local level of the one or more inflammatory molecules before local administration.
Embodiment 145: The method according to Embodiments 50, 104, or 120 wherein the pharmaceutical composition or population is effective to decrease the local anatomic tissue/fluid level of one or more pro-inflammatory molecules upon localized (e.g., by direct injection) administration to a lesional site in a subject by at least 5%, 10%, 25%, 50%, 80% or 90% relative to the local level of the one or more inflammatory molecules before the local administration.
Embodiment 146: The method according to Embodiment 80, wherein the conditioned media comprises one or more of microvesicles and exosomes.
Embodiment 147: The method according to Embodiment 146, further comprising isolating the one or more of microvesicles and exosomes from the conditioned media.
Embodiment 148: The composition according to Embodiment 121, wherein the conditioned media comprises one or more of microvesicles and exosomes.

### LIST OF CITED DOCUMENTS

Abdi, R., R. Moore, S. Sakai, C. B. Donnelly, M. Mounayar, and R. Sackstein. 2015. HCELL Expression on Murine MSC Licenses Pancreatotropism and Confers Durable Reversal of Autoimmune Diabetes in NOD Mice. Stem Cells 33:1523-1531.
Aggarwal, S., and M. F. Pittenger. 2005. Human mesenchymal stem cells modulate allogeneic immune cell responses. Blood 105:1815-1822.
Ankrum, J., and J. M. Karp. 2010. Mesenchymal stem cell therapy: Two steps forward, one step back. Trends Mol Med 16:203-209.
Aruffo, A., I. Stamenkovic, M. Melnick, C. B. Underhill, and B. Seed. 1990. CD44 is the principal cell surface receptor for hyaluronate. Cell 61:1303-1313.
Batten, P., P. Sarathchandra, J. W. Antoniw, S. S. Tay, M. W. Lowdell, P. M. Taylor, and M. H. Yacoub. 2006. Human mesenchymal stem cells induce T cell anergy and downregulate T cell allo-responses via the TH2 pathway: relevance to tissue engineering human heart valves. Tissue engineering 12:2263-2273.
Baldo, B. A. Chimeric fusion proteins used for therapy: indications, mechanisms, and safety, Drug Safety. 38 (5): 455-79, 2015
Beilhack, A., S. Schulz, J. Baker, G. F. Beilhack, C. B. Wieland, E. I. Herman, E. M. Baker, Y. A. Cao, C. H. Contag, and R. S. Negrin. 2005. In vivo analyses of early events in acute graft-versus-host disease reveal sequential infiltration of T-cell subsets. Blood 106:1113-1122.
Belkaid, Y., K. F. Hoffmann, S. Mendez, S. Kamhawi, M. C. Udey, T. A. Wynn, and D. L. Sacks. 2001. The role of interleukin (IL)-10 in the persistence of Leishmania major in the skin after healing and the therapeutic potential of anti-IL-10 receptor antibody for sterile cure. J Exp Med 194:1497-1506.
Burdick, M. M., J. T. Chu, S. Godar, and R. Sackstein. 2006. HCELL is the major E- and L-selectin ligand expressed on LS174T colon carcinoma cells. J Biol Chem 281:13899-13905.
Burman, A. C., T. Banovic, R. D. Kuns, A. D. Clouston, A. C. Stanley, E. S. Morris, V. Rowe, H. Bofinger, R. Skoczylas, N. Raffelt, O. Fahy, S. R. McColl, C. R. Engwerda, K. P. McDonald, and G. R. Hill. 2007. IFNgamma differentially controls the development of idiopathic pneumonia syndrome and GVHD of the gastrointestinal tract. Blood 110:1064-1072.
Casucci M. et al., CD44v6-targeted T cells mediate potent antitumor effects against acute myeloid leukemia and multiple myeloma, Blood. 2013 Nov. 14; 122(20):3461-72.
Couper, K. N., D. G. Blount, and E. M. Riley. 2008. IL-10: the master regulator of immunity to infection. J Immunol 180:5771-5777.
Cho, D. I., H. J. Kang, J. H. Jeon, G. H. Eom, H. H. Cho, M. R. Kim, M. Cho, H. Y. Jeong, H. C. Cho, M. H. Hong, Y. S. Kim, and Y. Ahn. 2019. Antiinflammatory activity of ANGPTL4 facilitates macrophage polarization to induce cardiac repair. JCI insight 4:
de Waal Malefyt, R., J. Haanen, H. Spits, M. G. Roncarolo, A. te Velde, C. Figdor, K. Johnson, R. Kastelein, H. Yssel, and J. E. de Vries. 1991. Interleukin 10 (IL-10) and viral IL-10 strongly reduce antigen-specific human T cell proliferation by diminishing the antigen-presenting capacity of monocytes via downregulation of class II major histocompatibility complex expression. J Exp Med 174:915-924.
Fiorentino, D. F., M. W. Bond, and T. R. Mosmann. 1989. Two types of mouse T helper cell. IV. Th2 clones secrete a factor that inhibits cytokine production by Th1 clones. J Exp Med 170:2081-2095.
Fiorentino, D. F., A. Zlotnik, P. Vieira, T. R. Mosmann, M. Howard, K. W. Moore, and A. O'Garra. 1991. IL-10 acts on the antigen-presenting cell to inhibit cytokine production by Th1 cells. J Immunol 146:3444-3451.
Fox, F. E., H. C. Ford, R. Douglas, S. Cherian, and P. C. Nowell. 1993. Evidence that TGF-beta can inhibit human T-lymphocyte proliferation through paracrine and autocrine mechanisms. Cellular immunology 150:45-58.
Francois, M., R. Romieu-Mourez, M. Li, and J. Galipeau. 2012. Human MSC suppression correlates with cytokine induction of indoleamine 2,3-dioxygenase and bystander M2 macrophage differentiation. Mol Ther 20:187-195.
Galipeau, J., and L. Sensebe. 2018. Mesenchymal Stromal Cells: Clinical Challenges and Therapeutic Opportunities. Cell Stem Cell 22:824-833.
Gatza, E., C. E. Rogers, S. G. Clouthier, K. P. Lowler, I. Tawara, C. Liu, P. Reddy, and J. L. Ferrara. 2008. Extracorporeal photopheresis reverses experimental graft-versus-host disease through regulatory T cells. Blood 112:1515-1521.
Groh, M. E., B. Maitra, E. Szekely, and O. N. Koc. 2005. Human mesenchymal stem cells require monocyte-mediated activation to suppress alloreactive T cells. Exp Hematol 33:928-934.
Hill, G. R., J. M. Crawford, K. R. Cooke, Y. S. Brinson, L. Pan, and J. L. Ferrara. 1997. Total body irradiation and acute graft-versus-host disease: the role of gastrointestinal damage and inflammatory cytokines. Blood 90:3204-3213.
Jiang, D., J. Liang, and P. W. Noble. 2011. Hyaluronan as an immune regulator in human diseases. Physiol Rev 91:221-264.
Levy O, et al. mRNA-engineered mesenchymal stem cells for targeted delivery of interleukin-10 to sites of inflammation. Blood. 2013; 122:e22-e32.
Li, C. Y., X. Y. Wu, J. B. Tong, X. X. Yang, J. L. Zhao, Q. F. Zheng, G. B. Zhao, and Z. J. Ma. 2015. Comparative analysis of human mesenchymal stem cells from bone marrow and adipose tissue under xeno-free conditions for cell therapy. Stem Cell Res Ther 6:55.
Lim, J. Y., K. I. Im, E. S. Lee, N. Kim, Y. S. Nam, Y. W. Jeon, and S. G. Cho. 2016. Enhanced immunoregulation of mesenchymal stem cells by IL-10-producing type 1 regulatory T cells in collagen-induced arthritis. Sci Rep 6:26851.
Lowe, J. B., and P. A. Ward. 1997. Therapeutic inhibition of carbohydrate-protein interactions in vivo. The Journal of clinical investigation 99:822-826.
Macatonia, S. E., T. M. Doherty, S. C. Knight, and A. O'Garra. 1993. Differential effect of IL-10 on dendritic cell-induced T cell proliferation and IFN-gamma production. J Immunol 150:3755-3765.
Manieri, N. A., M. R. Mack, M. D. Himmelrich, D. L. Worthley, E. M. Hanson, L. Eckmann, T. C. Wang, and T. S. Stappenbeck. 2015. Mucosally transplanted mesenchymal stem cells stimulate intestinal healing by promoting angiogenesis. The Journal of clinical investigation 125:3606-3618.
Meisel, R., A. Zibert, M. Laryea, U. Gobel, W. Daubener, and D. Dilloo. 2004. Human bone marrow stromal cells inhibit allogeneic T-cell responses by indoleamine 2,3-dioxygenase-mediated tryptophan degradation. Blood 103:4619-4621.
Miranda, K. M., M. G. Espey, and D. A. Wink. 2001. A rapid, simple spectrophotometric method for simultaneous detection of nitrate and nitrite. Nitric Oxide 5:62-71.
Najar, M., G. Raicevic, H. Fayyad-Kazan, C. De Bruyn, D. Bron, M. Toungouz, and L. Lagneaux. 2015. Bone Marrow Mesenchymal Stromal Cells Induce Proliferative, Cytokinic and Molecular Changes During the T Cell Response: The Importance of the IL-10/CD210 Axis. Stem cell reviews and reports 11:442-452.
Nauta, A. J., and W. E. Fibbe. 2007. Immunomodulatory properties of mesenchymal stromal cells. Blood 110:3499-3506.
Petrey, A. C., and C. A. de la Motte. 2014. Hyaluronan, a crucial regulator of inflammation. Front Immunol 5:101.
Rajbhandari, P., B. J. Thomas, A. C. Feng, C. Hong, J. Wang, L. Vergnes, T. Sallam, B. Wang, J. Sandhu, M. M. Seldin, A. J. Lusis, L. G. Fong, M. Katz, R. Lee, S. G. Young, K. Reue, S. T. Smale, and P. Tontonoz. 2018. IL-10 Signaling Remodels Adipose Chromatin Architecture to Limit Thermogenesis and Energy Expenditure. Cell 172:218-233 e217.
Reddy, P. 2003. Pathophysiology of acute graft-versus-host disease. Hematol Oncol 21:149-161.
Ren, G., L. Zhang, X. Zhao, G. Xu, Y. Zhang, A. I. Roberts, R. C. Zhao, and Y. Shi. 2008. Mesenchymal stem cell-mediated immunosuppression occurs via concerted action of chemokines and nitric oxide. Cell Stem Cell 2:141-150.
Robb, R. J., E. Kreijveld, R. D. Kuns, Y. A. Wilson, S. D. Olver, A. L. Don, N. C. Raffelt, N. A. De Weerd, K. E. Lineburg, A. Varelias, K. A. Markey, M. Koyama, A. D. Clouston, P. J. Hertzog, K. P. Macdonald, and G. R. Hill. 2011. Type I-IFNs control GVHD and GVL responses after transplantation. Blood 118:3399-3409.
Sackstein, R. 2006. A revision of Billingham's tenets: the central role of lymphocyte migration in acute graft-versus-host disease. Biol Blood Marrow Transplant 12:2-8.
Sackstein, R. 2009. Glycosyltransferase-programmed stereosubstitution (GPS) to create HCELL: engineering a roadmap for cell migration. Immunol Rev 230:51-74.
Sackstein, R. 2016. Fulfilling Koch's postulates in glycoscience: HCELL, GPS and translational glycobiology. Glycobiology 26:560-570.
Sackstein, R., J. S. Merzaban, D. W. Cain, N. M. Dagia, J. A. Spencer, C. P. Lin, and R. Wohlgemuth. 2008. Ex vivo glycan engineering of CD44 programs human multipotent mesenchymal stromal cell trafficking to bone. Nat Med 14:181-187.
Saraiva, M., P. Vieira, and A. O'Garra. 2020. Biology and therapeutic potential of interleukin-10. J Exp Med 217:
Sato, K., K. Ozaki, I. Oh, A. Meguro, K. Hatanaka, T. Nagai, K. Muroi, and K. Ozawa. 2007. Nitric oxide plays a critical role in suppression of T-cell proliferation by mesenchymal stem cells. Blood 109:228-234.
Schwab, L., L. Goroncy, S. Palaniyandi, S. Gautam, A. Triantafyllopoulou, A. Mocsai, W. Reichardt, F. J. Karlsson, S. V. Radhakrishnan, K. Hanke, A. Schmitt-Graeff, M. Freudenberg, F. D. von Loewenich, P. Wolf, F. Leonhardt, N. Baxan, D. Pfeifer, O. Schmah, A. Schonle, S. F. Martin, R. Mertelsmann, J. Duyster, J. Finke, M. Prinz, P. Henneke, H. Hacker, G. C. Hildebrandt, G. Hacker, and R. Zeiser. 2014. Neutrophil granulocytes recruited upon translocation of intestinal bacteria enhance graft-versus-host disease via tissue damage. Nat Med 20:648-654.
Skelton, T. P., C. Zeng, A. Nocks, and I. Stamenkovic. 1998. Glycosylation provides both stimulatory and inhibitory effects on cell surface and soluble CD44 binding to hyaluronan. J Cell Biol 140:431-446.
Sloane, J. P., and J. Norton. 1993. The pathology of bone marrow transplantation. Histopathology 22:201-209.
Socie, G., J. Y. Mary, M. Lemann, M. Daneshpouy, P. Guardiola, V. Meignin, L. Ades, H. Esperou, P. Ribaud, A. Devergie, E. Gluckman, J. C. Ameisen, and A. Janin. 2004. Prognostic value of apoptotic cells and infiltrating neutrophils in graft-versus-host disease of the gastrointestinal tract in humans: TNF and Fas expression. Blood 103:50-57.
Sotiropoulou, P. A., S. A. Perez, A. D. Gritzapis, C. N. Baxevanis, and M. Papamichail. 2006. Interactions between human mesenchymal stem cells and natural killer cells. Stem Cells 24:74-85.
Stappenbeck, T. S., and H. Miyoshi. 2009. The role of stromal stem cells in tissue regeneration and wound repair. Science 324:1666-1669.
Taga, K., and G. Tosato. 1992. IL-10 inhibits human T cell proliferation and IL-2 production. J Immunol 148:1143-1148.
Thankamony, S. P., and R. Sackstein. 2011. Enforced hematopoietic cell E- and L-selectin ligand (HCELL) expression primes transendothelial migration of human mesenchymal stem cells. Proc Natl Acad Sci USA 108:2258-2263.
Tormin, A., J. C. Brune, E. Olsson, J. Valcich, U. Neuman, T. Olofsson, S. E. Jacobsen, and S. Scheding. 2009. Characterization of bone marrow-derived mesenchymal stromal cells (MSC) based on gene expression profiling of functionally defined MSC subsets. Cytotherapy 11:114-128.
Uccelli, A., L. Moretta, and V. Pistoia. 2008. Mesenchymal stem cells in health and disease. Nat Rev Immunol 8:726-736.
Valencia, J., B. Blanco, R. Yanez, M. Vazquez, C. Herrero Sanchez, M. Fernandez-Garcia, C. Rodriguez Serrano, D. Pescador, J. F. Blanco, M. Hernando-Rodriguez, F. Sanchez-Guijo, M. L. Lamana, J. C. Segovia, A. Vicente, C. Del Canizo, and A. G. Zapata. 2016. Comparative analysis of the immunomodulatory capacities of human bone marrow- and adipose tissue-derived mesenchymal stromal cells from the same donor. Cytotherapy 18:1297-1311.
Vieira, P., R. de Waal-Malefyt, M. N. Dang, K. E. Johnson, R. Kastelein, D. F. Fiorentino, J. E. deVries, M. G. Roncarolo, T. R. Mosmann, and K. W. Moore. 1991. Isolation and expression of human cytokine synthesis inhibitory factor cDNA clones: homology to Epstein-Barr virus open reading frame BCRFI. Proc Natl Acad Sci USA 88:1172-1176.
Warren L, Manos P D, Ahfeldt T et al. Highly efficient reprogramming to pluripotency and directed differentiation of human cells with synthetic modified mRNA. Cell Stem Cell. 2010; 7:618-630.
Wysocki, C. A., A. Panoskaltsis-Mortari, B. R. Blazar, and J. S. Serody. 2005. Leukocyte migration and graft-versus-host disease. Blood 105:4191-4199.
Yanez, R., M. L. Lamana, J. Garcia-Castro, I. Colmenero, M. Ramirez, and J. A. Bueren. 2006. Adipose tissue-derived mesenchymal stem cells have in vivo immunosuppressive properties applicable for the control of the graft-versus-host disease. Stem Cells 24:2582-2591.
Yanez, R., A. Oviedo, M. Aldea, J. A. Bueren, and M. L. Lamana. 2010. Prostaglandin E2 plays a key role in the immunosuppressive properties of adipose and bone marrow tissue-derived mesenchymal stromal cells. Exp Cell Res 316:3109-3123.
Ziegler, T. R., A. Panoskaltsus-Mortari, L. H. Gu, C. R. Jonas, C. L. Farrell, D. L. Lacey, D. P. Jones, and B. R. Blazar. 2001. Regulation of glutathione redox status in lung and liver by conditioning regimens and keratinocyte growth factor in murine allogeneic bone marrow transplantation. Transplantation 72:1354-1362.

1. A pharmaceutical composition comprising a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells.

2. A pharmaceutical composition comprising conditioned media obtained from a population of CD44⁺ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD44⁺ cells.

3. The pharmaceutical composition of claim 1 wherein the population of CD44+ cells is CD34⁻/PSGL⁻, wherein the modified cells of (1) or (2) produce elevated levels of IL-10 and at least one additional anti-inflammatory molecule relative to a native population of CD34⁻/CD44⁺/PSGL⁻ cells.

4. A pharmaceutical composition comprising conditioned media obtained from a population of CD34⁻/CD44⁺/PSGL⁻ cells that have been (1) modified ex vivo via exofucosylation to enforce hematopoietic cell E-Selectin/L-Selectin Ligand (HCELL) expression and treated with E-selectin or L-selectin for a period of time sufficient to prime the cells to produce interleukin-10 (IL-10) and at least one additional anti-inflammatory molecule; or (2) modified ex vivo via treatment with hyaluronic acid (HA) for a period of time sufficient to prime the cells to produce interleukin-10 (IL-10) and at least one additional anti-inflammatory molecule, wherein the modified cells of (1) or (2) produce elevated levels of IL-10 and at least one additional anti-inflammatory molecule relative to a native population of CD34⁻/CD44⁺/PSGL⁻ cells.

5. The pharmaceutical composition according to claim 1 wherein the population of cells are mesenchymal stem cells (MSCs), hematopoietic stem cells, tissue stem/progenitor cells (for example, a neural stem cell, myocyte stem cell or pulmonary stem cell), stromal vascular fraction cells, umbilical cord-derived stem cells, or embryonic stem cells, induced pluripotent stem cells, differentiated progenitors derived from embryonic stem cells or from induced pluripotent stem cells, differentiated progenitors derived from adult stem cells, primary cells isolated from any tissue (e.g., blood, bone marrow, brain, liver, lung, gut, stomach, fat, muscle, testes, uterus, ovary, skin, spleen, eye, endocrine organ and bone), a culture-expanded progenitor cell population, a culture-expanded stem cell population, or a culture-expanded primary cell population.

6. (canceled)

7. The pharmaceutical composition according to claim 1 wherein the population of cells are culture-expanded mesenchymal stem cells.

8.-10. (canceled)

11. The pharmaceutical composition according to claim 1 wherein the at least one additional anti-inflammatory molecule is selected from TGF-β, IDO, nitric oxide (NO) metabolites, PGE₂ and combinations thereof.

12. The pharmaceutical composition according to claim 1, wherein the IL-10 production is elevated at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, or at least 100-fold relative to a native population of the cells.

13. (canceled)

14. (canceled)

15. The pharmaceutical composition according to claim 1 useful for decreasing plasma levels of at least one pro-inflammatory molecule in a subject when administered to the subject.

16. The pharmaceutical composition according to claim 15, wherein the at least one pro-inflammatory molecule comprises a group selected from IFNγ, TNFα, IL-1α, IL-1β, IL-6, IL-12, IL-17 and combinations thereof.

17. The pharmaceutical composition according to claim 1 wherein the E-Selectin or L-selectin is an E-Selectin-immunoglobulin or L-selectin-immunoglobulin chimera (E-Ig chimera or L-Ig chimera).

18. The pharmaceutical composition according to claim 1 useful for the treatment of a disease associated with one or more of neoplasia (e.g., breast cancer, lung cancer, prostate cancer, lymphoma, leukemia, etc.), immunologic/autoimmune conditions (e.g., graft vs. host disease, multiple sclerosis, diabetes, inflammatory bowel disease, lupus erythematosus, rheumatoid arthritis, psoriasis, vasculitides, etc.), direct tissue injury (e.g., burns, trauma, decubitus ulcers, etc.), ischemic/vascular events (e.g., myocardial infarct, stroke, shock, hemorrhage, coagulopathy, etc.), infections (e.g., cellulitis, pneumonia, meningitis, sepsis, systemic inflammatory response syndrome, acute respiratory disease syndrome secondary to bacteria, fungi or viruses (e.g., influenza, coronavirus, COVID-19, SARS, MERS, etc.), degenerative diseases (e.g., osteoporosis, osteoarthritis, Alzheimer's disease, etc.), congenital/genetic diseases (e.g., epidermolysis bullosa, osteogenesis imperfecta, muscular dystrophies, lysosomal storage diseases, Huntington's disease, etc.), adverse drug effects (e.g., drug-induced hepatitis, drug-induced cardiac injury, etc.), toxic injuries (e.g., radiation exposure(s), chemical exposure(s), alcoholic hepatitis, alcoholic pancreatitis, alcoholic cardiomyopathy, cocaine cardiomyopathy, etc.), metabolic derangements (e.g., uremic pericarditis, metabolic acidosis, etc.), iatrogenic conditions (e.g., radiation-induced tissue injury, surgery-related complications, etc.), and/or idiopathic processes (e.g., amyotrophic lateral sclerosis, Parsonnage-Turner Syndrome, etc.).

19. The pharmaceutical composition according to claim 1 useful for the treatment of a disease associated with a cytokine storm.

20. The pharmaceutical composition according to claim 1 useful for engendering immunohomeostasis in a subject.

21. The pharmaceutical composition according to claim 1 useful for the treatment of graft versus host (GvH) disease.

22.-139. (canceled)

140. The pharmaceutical composition according to claim 2, wherein the conditioned media comprises one or more of microvesicles, membrane particles, and exosomes.

150. The pharmaceutical composition according to claim 2 wherein the conditioned media is processed to isolate one or more of microvesicles, membrane particles, and exosomes.

151. The composition according to claim 1, wherein the population of CD44⁺ cells is effective to produce one or more of microvesicles, membrane particles, and exosomes.

152. The pharmaceutical composition according to claim 4, wherein the conditioned media comprises one or more of microvesicles, membrane particles, and exosomes.

153. The pharmaceutical composition of claim 4, wherein the conditioned media is processed to isolate the one or more of microvesicles, membrane particles, and exosomes.


SEQUENCE LISTING (12 sequences)

SEQ ID NO: 1 - protein, 361 aa, Homo sapiens
MDPLGAAKPQWPWRRCLAALLFQLLVAVCFFSYLRVSRDDATGSPRAPSGSSRQDTTPTRPTLLILLWTWPFHIPVALSRCSEMVPGTADCHITADRKVYPQADTVIVHHWDIMSNPKSRLPPSPRPQGQRWIWFNLEPPPNCQHLEALDRYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALDFCKACWKLQQESRYQTVRSIAAWFT

SEQ ID NO: 2 - DNA, 1694 bp, Homo sapiens
  source 1..1694: mol_type = genomic DNA
gagaaagcaggcaaccaccatgtcatttgaaaacagtttcatcgggatataattcgcaacccatacagtgaatccatttaagatactctgacccatggatcccctgggtgcagccaagccacaatggccatggcgccgctgtctggccgcactgctatttcagctgctggtggctgtgtgtttcttctcctacctgcgtgtgtcccgagacgatgccactggatcccctagggctcccagtgggtcctcccgacaggacaccactcccacccgccccaccctcctgatcctgctatggacatggcctttccacatccctgtggctctgtcccgctgttcagagatggtgcccggcacagccgactgccacatcactgccgaccgcaaggtgtacccacaggcagacacggtcatcgtgcaccactgggatatcatgtccaaccctaagtcacgcctcccaccttccccgaggccgcaggggcagcgctggatctggttcaacttggagccaccccctaactgccagcacctggaagccctggacagatacttcaatctcaccatgtcctaccgcagcgactccgacatcttcacgccctacggctggctggagccgtggtccggccagcctgcccacccaccgctcaacctctcggccaagaccgagctggtggcctgggcggtgtccaactggaagccggactcagccagggtgcgctactaccagagcctgcaggctcatctcaaggtggacgtgtacggacgctcccacaagcccctgcccaaggggaccatgatggagacgctgtcccggtacaagttctacctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggacgacttccagagccccaaggacctggcccggtacctgcaggagctggacaaggaccacgcccgctacctgagctactttcgctggcgggagacgctgcggcctcgctccttcagctgggcactggatttctgcaaggcctgctggaaactgcagcaggaatccaggtaccagacggtgcgcagcatagcggcttggttcacctgagaggccggcatggtgcctgggctgccgggaacctcatctgcctggggcctcacctgctggagtcctttgtggccaaccctctctcttacctgggacctcacacgctgggcttcacggctgccaggagcctctcccctccagaagacttgcctgctggggacctcgcctgctggggacctcgcctgttggggacctcacctgctggggacctcacctgctggggaccttggctgctggaggctgcacctactgaggatgtcggcggtcggggactttacctgctgggacctgctcccagagaccttgccacactgaatctcacctgctggggacctcaccctggagggccctgggccctggggaactggcttacttggggccccacccgggagtgatggttctggctgatttgtttgtgatgttgttagccgcctgtgaggggtgcagagagatcatcacggcacggtttccagatgtaatactgcaaggaaaaaaaaaaaaaaaaaaaaaaaaa

SEQ ID NO: 3 - protein, 530 aa, Homo sapiens
MRRLWGAARKPSGAGWEKEWAEAPQEAPGAWSGRLGPGRSGRKGRAVPGWASWPAHLALAARPARHLGGAGQGPRPLHSGTAPFHSRASGERQRRLEPQLQHESRCRSSTPADAWRAEAALPVRAMGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 4 - DNA, 2287 bp, Homo sapiens
  source 1..2287: mol_type = genomic DNA
gcggcagctgctttagaaggtctcgagcctcctgtaccttcccagggatgaaccgggccttccctctggaaggcgagggttcgggccacagtgagcgagggccagggcggtgggcgcgcgcagagggaaaccggatcagttgagagagaatcaagagtagcggatgaggcgcttgtggggcgcggcccggaagccctcgggcgcgggctgggagaaggagtgggcggaggcgccgcaggaggctcccggggcctggtcgggccggctgggccccgggcgcagtggaagaaagggacgggcggtgcccggttgggcgtcctggccagctcaccttgccctggcggctcgccccgcccggcacttgggaggagcagggcagggcccgcggcctttgcattctgggaccgcccccttccattcccgggccagcggcgagcggcagcgacggctggagccgcagctacagcatgagagccggtgccgctcctccacgcctgcggacgcgtggcgagcggaggcagcgctgcctgttcgcgccatgggggcaccgtggggctcgccgacggcggcggcgggcgggcggcgcgggtggcgccgaggccgggggctgccatggaccgtctgtgtgctggcggccgccggcttgacgtgtacggcgctgatcacctacgcttgctgggggcagctgccgccgctgccctgggcgtcgccaaccccgtcgcgaccggtgggcgtgctgctgtggtgggagcccttcggggggcgcgatagcgccccgaggccgccccctgactgccggctgcgcttcaacatcagcggctgccgcctgctcaccgaccgcgcgtcctacggagaggctcaggccgtgcttttccaccaccgcgacctcgtgaaggggccccccgactggcccccgccctggggcatccaggcgcacactgccgaggaggtggatctgcgcgtgttggactacgaggaggcagcggcggcggcagaagccctggcgacctccagccccaggcccccgggccagcgctgggtttggatgaacttcgagtcgccctcgcactccccggggctgcgaagcctggcaagtaacctcttcaactggacgctctcctaccgggcggactcggacgtctttgtgccttatggctacctctaccccagaagccaccccggcgacccgccctcaggcctggccccgccactgtccaggaaacaggggctggtggcatgggtggtgagccactgggacgagcgccaggcccgggtccgctactaccaccaactgagccaacatgtgaccgtggacgtgttcggccggggcgggccggggcagccggtgcccgaaattgggctcctgcacacagtggcccgctacaagttctacctggctttcgagaactcgcagcacctggattatatcaccgagaagctctggcgcaacgcgttgctcgctggggcggtgccggtggtgctgggcccagaccgtgccaactacgagcgctttgtgccccgcggcgccttcatccacgtggacgacttcccaagtgcctcctccctggcctcgtacctgcttttcctcgaccgcaaccccgcggtctatcgccgctacttccactggcgccggagctacgctgtccacatcacctccttctgggacgagccttggtgccgggtgtgccaggctgtacagagggctggggaccggcccaagagcatacggaacttggccagctggttcgagcggtgaagccgcgctcccctggaagcgacccaggggaggccaagttgtcagctttttgatcctctactgtgcatctccttgactgccgcatcatgggagtaagttcttcaaacacccatttttgctctatgggaaaaaaacgatttaccaattaatattactcagcacagagatgggggcccggtttccatattttttgcacagctagcaattgggctccctttgctgctgatgggcatcattgtttaggggtgaaggagggggttcttcctcaccttgtaaccagtgcagaaatgaaatagcttagcggcaagaagccgttgaggcggtttcctgaatttccccatctgccacaggccatatttgtggcccgtgcagcttccaaatctcatacacaactgttcccgattcacgtttttctggaccaaggtgaagcaaatttgtggttgtagaaggagccttgttggtggagagtggaaggactgtggctgcaggtgggactttgttgtttggattcctcacagccttggctcctga

SEQ ID NO: 5 - protein, 405 aa, Homo sapiens
MGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 6 - DNA, 1215 bp, Homo sapiens
  source 1..1215: mol_type = genomic DNA
atgggggcaccgtggggctcgccgacggcggcggcgggcgggcggcgcgggtggcgccgaggccgggggctgccatggaccgtctgtgtgctggcggccgccggcttgacgtgtacggcgctgatcacctacgcttgctgggggcagctgccgccgctgccctgggcgtcgccaaccccgtcgcgaccggtgggcgtgctgctgtggtgggagcccttcggggggcgcgatagcgccccgaggccgccccctgactgccggctgcgcttcaacatcagcggctgccgcctgctcaccgaccgcgcgtcctacggagaggctcaggccgtgcttttccaccaccgcgacctcgtgaaggggccccccgactggcccccgccctggggcatccaggcgcacactgccgaggaggtggatctgcgcgtgttggactacgaggaggcagcggcggcggcagaagccctggcgacctccagccccaggcccccgggccagcgctgggtttggatgaacttcgagtcgccctcgcactccccggggctgcgaagcctggcaagtaacctcttcaactggacgctctcctaccgggcggactcggacgtctttgtgccttatggctacctctaccccagaagccaccccggcgacccgccctcaggcctggccccgccactgtccaggaaacaggggctggtggcatgggtggtgagccactgggacgagcgccaggcccgggtccgctactaccaccaactgagccaacatgtgaccgtggacgtgttcggccggggcgggccggggcagccggtgcccgaaattgggctcctgcacacagtggcccgctacaagttctacctggctttcgagaactcgcagcacctggattatatcaccgagaagctctggcgcaacgcgttgctcgctggggcggtgccggtggtgctgggcccagaccgtgccaactacgagcgctttgtgccccgcggcgccttcatccacgtggacgacttcccaagtgcctcctccctggcctcgtacctgcttttcctcgaccgcaaccccgcggtctatcgccgctacttccactggcgccggagctacgctgtccacatcacctccttctgggacgagccttggtgccgggtgtgccaggctgtacagagggctggggaccggcccaagagcatacggaacttggccagctggttcgagcgg

SEQ ID NO: 7 - protein, 374 aa, Homo sapiens
MDPLGPAKPQWLWRRCLAGLLFQLLVAVCFFSYLRVSRDDATGSPRPGLMAVEPVTGAPNGSRCQDSMATPAHPTLLILLWTWPFNTPVALPRCSEMVPGAADCNITADSSVYPQADAVIVHHWDIMYNPSANLPPPTRPQGQRWIWFSMESPSNCRHLEALDGYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQQESRYQTVRSIAAWFT

SEQ ID NO: 8 - DNA, 1487 bp, Homo sapiens
  source 1..1487: mol_type = genomic DNA
acagacatgagccacctcgcccagccttttcatggctgagtgatattccattgtgtggatggatcacactagctactctgacccatggatcccctgggcccagccaagccacagtggctgtggcgccgctgtctggccgggctgctgtttcagctgctggtggctgtgtgtttcttctcctacctgcgtgtgtcccgagacgatgccactggatcccctaggccagggcttatggcagtggaacctgtcaccggggctcccaatgggtcccgctgccaggacagcatggcgacccctgcccaccccaccctactgatcctgctgtggacgtggccttttaacacacccgtggctctgccccgctgctcagagatggtgcccggcgcggccgactgcaacatcactgccgactccagtgtgtacccacaggcagacgcggtcatcgtgcaccactgggatatcatgtacaaccccagtgccaacctcccgccccccaccaggccgcaggggcagcgctggatctggttcagcatggagtcccccagcaactgccggcacctggaagccctggacggatacttcaatctcaccatgtcctaccgcagcgactccgacatcttcacgccctatggctggctggagccgtggtccggccagcctgcccacccaccgctcaacctctcggccaagaccgagctggtggcctgggcggtgtccaactggaagccggactcggccagggtgcgctactaccagagcctgcaggctcatctcaaggtggacgtgtacggacgctcccacaagcccctgcccaaggggaccatgatggagacgctgtcccggtacaagttctatctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggatgacttccagagccccaaggacctggcccggtacctgcaggagctggacaaggaccacgcccgctacctgagctactttcgctggcgggagacgctgcggcctcgctccttcagctgggcactggctttctgcaaggcctgctggaagctgcagcaggaatccaggtaccagacggtgcgcagcatagcggcttggttcacctgagaggccggcatggggcctgggctgccaggacctcactttcccagggcctcacctacctagggtctcactagtcgggggatttacctacctggggcctcggctgcctggggtctcacctgcctggggcctcacctgctggagtctttggtggccaggcatgtgacttacctgggatttcacttgccgggcttcactgccaggagcctcccctgctggggaccttgccagctggggctggggatggtgcctactggggaccttgctttctggaggctgca

SEQ ID NO: 9 - protein, 359 aa, Homo sapiens [sequence omitted]

SEQ ID NO: 10 - DNA, 2875 bp, Homo sapiens
  source 1..2875: mol_type = genomic DNA
ggggagatcatttctctccattccaggaagtttgcatagctccctggacttctgctttgcactgccctgcaggagtgggtggggaaaggaagtggctttgaggcacacagaggggcttggtgaggccaccggaggaagcttctgccaccaatatgggacctgtgcccagcctaccagaagagagcatctgaaaacatgtatcgacatggtaacccctctgcttgaagcctcacatggctccctattgccttggtgctgaacaccctatggctgaccgtggcccagcctctgcaacagctctgcctcctctccagtggtgaagacccagcctgctgagactcctcctgcagttcctcaacatgcctgcatttctgctgcctcagggcctttgcgaaggttgttccttgtaactggaatgcccttccatcccttttttattcaaaaggctgcaattttaattgaagaaagttcccttccaaggttcatgagttgcctgacttgcccaccggtttcctgcaagatcccttggcctggcacttagtgctcaggaaatatttggtgatgggccaactgagtgagaaggtgggatctggtgggaaggaaagcggaaaggtagaaattctgctcacttcctcattcccacctcccaaggaacccctggtgtccctgtggaacccgctttgggaaccggtggttcaggtcagccttttcactttgtactcaaagccacatcgcattgaagccacaggtggggcaaggtcatgcatgacctgagtctccaaatcccttcaccctgtttggttctgcaacggggattaggggagccccacgatttgttttcaaaggatgtccgggctccaggacaggatgccctgggtcacctgatgacaggtgtggtggttggaaagggcctggtttcagctccgggtacacttcctccttccttctgctgcgtggtgtggcctcttccacgtcctcagaatccagctgttactccgtccgcggcctctcagctctagggccctctgcacactggcccccccagatactctgacccatggatcccctgggcccggccaagccacagtggtcgtggcgctgctgtctgaccacgctgctgtttcacctgctgatggctgtgtgtttcttctcctatctgcgtgtgtctcaagacgatcccactgtgtaccctaatgggtcccgcttcccagacagcacagggacccccgcccactccatccccctgatcctgctgtggacgtggccttttaacaaacccatagctctgccccgctgctcagagatggtgcctggcacggctgactgcaacatcactgccgaccgcaaggtgtatccacaggcagacgcggtcatcgtgcaccaccgagaggtcatgtacaaccccagtgcccagctcccacgctcctcgaggcggcaggggcagcgatggatctggttcagcatggagtccccaagccactgctggcagctgaaagccatggacggatacttcaatctcaccatgtcctaccgcagcgactccgacatcttcacgccctacggctggctggagccgtggtccggccagcctgcccacccaccgctcaacctctcggccaagaccgagctggtggcctgggcagtgtccaactgggggccaaactccgccagggtgcgctactaccagagcctgcaggcccatctcaaggtggacgtgtacggacgctcccacaagcccctgccccagggaaccatgatggagacgctgtcccggtacaagttctatctggccttcaagaactccttgcaccccgactacatcaccgagaagctgtggaggaacgccctggaggcctgggccgtgcccgtggtgctgggccccagcagaagcaactacgagaggttcctgccgcccgacgccttcatccacgtggacgacttccagagccccaaggacctggcccggtacctgcaggagctggacaaggaccacgcccgctacctgagctactttcgctggcgggagacgctgcggcctcgctccttcagctgggcactcgctttctgcaaggcctgctggaaactgcaggaggaatccaggtaccagacacgcggcatagcggcttggttcacctgagaggctggtgtggggcctgggctgccaggaacctcattttcctggggcctcacctgagtgggggcctcatctacctaaggactcgtttgcctgaagcttcacctgcctgaggactcacctgcctgggacggtcacctgttgcagcttcacctgcctggggattcacctacctgggtcctcactttcctggggcctcacctgctggagtcttcggtggccaggtatgtcccttacctgggatttcacatgctggcttccaggagcgtcccctgcggaagcctggcctgctggggatgtctcctggggactttgcctactggggacctcggctgttggggactttacctgctgggacctgctcccagagaccttccacactgaatctcacctgctaggagcctcacctgctggggacctcaccctggagggcactgggccctgggaactggcacccatggggccccacccatgagtgatggttctggctgatttgtttgtgatgttgttagccgcctgtgaggggtgcagagagataatcaccgcaccgtttccagatgtaatactgcaaagaaaaccgatgatgaggccgggtgcggtggctcacacctgtaatcccagcactttgggaggccgaggcaggcggatcacaaggtcaggagatcgagaccatcctggccaatatggtgaaacccgtctctactaaaaatacaaaaaaaaaaaaaaaaaaaaaaaaaaaaa

SEQ ID NO: 11 - protein, 342 aa, Homo sapiens
MNNAGHGPTRRLRGLGVLAGVALLAALWLLWLLGSAPRGTPAPQPTITILVWHWPFTDQPPELPSDTCTRYGIARCHLSANRSLLASADAVVFHHRELQTRRSHLPLAQRPRGQPWVWASMESPSHTHGLSHLRGIFNWVLSYRRDSDIFVPYGRLEPHWGPSPPLPAKSRVAAWVVSNFQERQLRARLYRQLAPHLRVDVFGRANGRPLCASCLVPTVAQYRFYLSFENSQHRDYITEKFWRNALVAGTVPVVLGPPRATYEAFVPADAFVHVDDFGSARELAAFLTGMNESRYQRFFAWRDRLRVRLFTDWRERFCAICDRYPHLPRSQVYEDLEGWFQA

SEQ ID NO: 12 - DNA, 1101 bp, Homo sapiens
  source 1..1101: mol_type = genomic DNA
gctgactgatcctgggagactgtggatgaataatgctgggcacggccccacccggaggctgcgaggcttgggggtcctggccggggtggctctgctcgctgccctctggctcctgtggctgctggggtcagcccctcggggtaccccggcaccccagcccacgatcaccatccttgtctggcactggcccttcactgaccagcccccagagctgcccagcgacacctgcacccgctacggcatcgcccgctgccacctgagtgccaaccgaagcctgctggccagcgccgacgccgtggtcttccaccaccgcgagctgcagacccggcggtcccacctgcccctggcccagcggccgcgagggcagccctgggtgtgggcctccatggagtctcctagccacacccacggcctcagccacctccgaggcatcttcaactgggtgctgagctaccggcgcgactcggacatctttgtgccctatggccgcctggagccccactgggggccctcgccaccgctgccagccaagagcagggtggccgcctgggtggtcagcaacttccaggagcggcagctgcgtgccaggctgtaccggcagctggcgcctcatctgcgggtggatgtctttggccgtgccaatggacggccactgtgcgccagctgcctggtgcccaccgtggcccagtaccgcttctacctgtcctttgagaactctcagcaccgcgactacattacggagaaattctggcgcaacgcactggtggctggcactgtgccagtggtgctggggcccccacgggccacctatgaggccttcgtgccggctgacgccttcgtgcatgtggatgactttggctcagcccgagagctggcggctttcctcactggcatgaatgagagccgataccaacgcttctttgcctggcgtgacaggctccgcgtgcgactgttcaccgactggcgggaacgtttctgtgccatctgtgaccgctacccacacctaccccgcagccaagtctatgaggaccttgagggttggtttcaggcctgagatccgctggccgggggaggtgggtgtgggtggaagggctgggtgtc